(19) United States
(12) Patent Application Publication
(10) Pub. No.: US 2026/0009802 A1
(43) Pub. Date: Jan. 8, 2026

(54) BIOMARKERS

(71) Applicants: OXFORD UNIVERSITY INNOVATION LIMITED, Oxford (GB); THE GENERAL HOSPITAL CORPORATION, Boston, MA (US)

(72) Inventors: Cornelia MARJA VAN DUIJN, Oxford (GB); Michael Austin ARGENTIERI, Boston, MA (US)

(21) Appl. No.: 18/761,477

(22) Filed: Jul. 2, 2024

(51) Int. Cl. G01N 33/68

(52) U.S. Cl. CPC G01N 33/6893; G01N 2333/075; G01N 2333/10; G01N 2333/4716; G01N 2333/4719; G01N 2333/4724; G01N 2333/4745; G01N 2333/475; G01N 2333/4756; G01N 2333/525; G01N 2333/54; G01N 2333/5756; G01N 2333/58; G01N 2333/70503; G01N 2333/70546; G01N 2333/71; G01N 2333/715; G01N 2333/7151; G01N 2333/7155; G01N 2333/7158; G01N 2333/78; G01N 2333/8139; G01N 2333/9029; G01N 2333/904; G01N 2333/908; G01N 2333/912; G01N 2333/916; G01N 2333/924; G01N 2333/96411; G01N 2333/96419; G01N 2333/96436; G01N 2333/96455; G01N 2333/96483; G01N 2333/988; G01N 2800/50; G01N 2800/7042

(57) ABSTRACT

The present invention relates to a method for determining, predicting or estimating the biological age of a subject, or for providing a measurement for use in determining, predicting or estimating the biological age of a subject or for predicting the presence or absence of at least one disease in a subject, predicting the risk of a subject of having or developing at least one disease; and/or predicting the risk of mortality of a subject. This invention also relates to a device for determining the presence and/or amount of each biomarker in a set of biomarkers; a set of probes for determining the presence or amount of a set of biomarkers, and the use of such device and/or probes in any of the above methods. Also provided is a biomarker testing kit for use in a method as described herein and a computer-readable storage medium or a computer program comprising computer-executable instructions and associated method.

### FIELD OF THE INVENTION

The present invention relates to a method for determining, predicting or estimating the biological age of a subject, or for providing a measurement for use in determining, predicting or estimating the biological age of a subject or for predicting the presence or absence of at least one disease in a subject, predicting the risk of a subject of having or developing at least one disease; and/or predicting the risk of mortality of a subject. This invention also relates to a device for determining the presence and/or amount of each biomarker in a set of biomarkers; a set of probes for determining the presence or amount of a set of biomarkers, and the use of such device and/or probes in any of the above methods. Also provided is a biomarker testing kit for use in a method as described herein.

### BACKGROUND

Age is a major determinant for most common chronic diseases and causes of death. Aging involves a progressive loss of physiological integrity and function over time, which ultimately leads to the development, and often co-occurrence, of major diseases and death. Incidence rates of major chronic diseases such as ischemic heart disease (IHD), stroke, diabetes, liver and kidney diseases, neurodegenerative diseases, and most cancers, all have varying rates of increasing risk with age, although there is substantial variation across individuals in the timing and severity of age-related disorders. Chronological age is a strong but imperfect surrogate measure of “biological” aging, which can be estimated more precisely by using ‘omics and other biomarker data, capturing the level of biological functioning of an individual in comparison to an expected level of functioning for a given chronological age.

How fast one ages not only determines individual risk of major chronic diseases and premature death, but also shapes the extent of morbidity and disability in the population, which has a major impact on health care systems. Further, the ability to quantify, and possibly intervene upon, biological aging may therefore have important consequences for prevention of multi-morbidity and premature death.

Biological aging is often measured using a biological aging clock which reflects the biological age of a subject by measurement of at least one biological or physiological parameter in said subject. Thus the clock can be used to compare the biological age and chronological age of a subject and assess whether a subject shows more or less evidence of aging biologically as compared to other persons with a similar chronological age. The utility of a biological aging clock depends on how well the clock predicts relevant outcomes for clinical care and public health, such as lifespan, risk of disease, and mortality.

A large number of biological aging clocks have previously been developed using DNA methylation (DNAm) (e.g., Rutledge et al. 2022, Horvath et al. 2018) or protein levels (e.g., Sayed et al. 2021, Oh et al. 2023).

U.S. Ser. No. 10/665,326B2 describes a method to predict the biological age of a tissue or organ, without establishing a link with disease occurrence or mortality. Sayed et al. (2021) have developed an inflammatory aging clock which focuses on cardiovascular disease prediction, while Oh et al. (2023) have developed a clock for disease prediction based on organ-specific proteomic data. Both clocks disclosed by Sayed et al. and Oh et al. are established based on a small number of persons and the clocks have been validated for a limited number of diseases and/or organs. Therefore there are limitations associated with the utility of these clocks.

The present invention seeks to overcome or ameliorate problems associated with methods of predicting biological age, risk of disease and risk of mortality in the art.

### BRIEF SUMMARY OF THE DISCLOSURE

The present invention is based upon the identification of biomarkers that can function as a biological clock and can predict disease occurrence and mortality based on biological age estimation. The clock has been established using a large general population sample, and has also been validated independently across diverse populations having different ethnic backgrounds. The clock predicts relevant outcomes for clinical care and public health, including biochemical and clinical risk factors, risk of disease and mortality.

In some embodiments, the present invention provides a method for determining, predicting or estimating the biological age of a subject, or for providing a measurement for use in determining, predicting or estimating the biological age of a subject, wherein the method comprises:
a) measuring, in a biological sample obtained from the subject at a first time point, the presence or amount of each biomarker in a set of biomarkers, wherein the set of biomarkers comprises at least 7 biomarkers selected from Table 1:

TABLE 1

| | |
|---|---|
| Acrosomal protein SP-10 | Glial fibrillary acidic protein |
| Agouti-related protein | Immunoglobulin superfamily DCC subclass |
| | member 4 |
| CUB domain-containing protein 1 | Prostate-specific antigen |
| Collagen alpha-3(VI) chain | Kallikrein-7 |
| C-X-C motif chemokine 17 | Leukocyte cell-derived chemotaxin-2 |
| Tumor necrosis factor receptor superfamily | Latent-transforming growth factor beta- |
| member 27 | binding protein 2 |
| Elastin | Neurofilament light polypeptide |
| Endoglin | Podocalyxin-like protein 2 |
| Follitropin subunit beta | Receptor-type tyrosine-protein phosphatase |
| | R |
| Growth/differentiation factor 15 | Scavenger receptor class F member 2 |

In some embodiments, the present invention provides a method for determining, predicting or estimating the biological age of a subject, or for providing a measurement for use in determining, predicting or estimating the biological age of a subject, wherein the method comprises:
a) measuring, in a biological sample obtained from the subject at a first time point, the presence or amount of each biomarker in a set of biomarkers, wherein the set of biomarkers comprises at least 50 biomarkers selected from Table 2:

TABLE 2

| | |
|---|---|
| Acrosomal protein SP-10 | PDZ domain-containing protein GIPC2 |
| Actin, aortic smooth muscle | Pancreatic secretory granule membrane |
| | major glycoprotein GP2 |
| Adenosine deaminase | Granzyme B |
| A disintegrin and metalloproteinase with | Hepatitis A virus cellular receptor 1 |
| thrombospondin motifs 13 | |
| A disintegrin and metalloproteinase with | Hemicentin-2 |
| thrombospondin motifs 15 | |
| A disintegrin and metalloproteinase with | Corticosteroid 11-beta-dehydrogenase |
| thrombospondin motifs 16 | isozyme 1 |
| ADAMTS-like protein 5 | Immunoglobulin superfamily DCC subclass |
| | member 4 |
| Adhesion G-protein coupled receptor G1 | Interleukin-17D |
| Alpha-fetoprotein | Interleukin-5 receptor subunit alpha |
| Advanced glycosylation end product-specific | Interleukin-7 receptor subunit alpha |
| receptor | |
| Agouti-related protein | Insulin-like 3 |
| Protein AHNAK2 | Integrin alpha-V |
| Angiopoietin-2 | Integrin beta-5 |
| BAG family molecular chaperone regulator 3 | Integrin beta-like protein 1 |
| Brevican core protein | Kinesin-like protein KIF22 |
| Osteocalcin | Mast/stem cell growth factor receptor Kit |
| Brother of CDO | Kallikrein-14 |
| Basigin | Prostate-specific antigen |
| Protein C19orf12 | Kallikrein-4 |
| Complement C1q-like protein 2 | Kallikrein-7 |
| Carbonic anhydrase 14 | Kallikrein-8 |
| Carbonic anhydrase 4 | Killer cell lectin-like receptor subfamily F |
| | member 1 |
| Calbindin | Neural cell adhesion molecule L1 |
| Coiled-coil domain-containing protein 80 | Extracellular glycoprotein lacritin |
| C-C motif chemokine 28 | Leukocyte cell-derived chemotaxin-2 |
| CCN family member 5 | Protein LEG1 homolog |
| T-cell surface glycoprotein CD1c | Lutropin subunit beta |
| Endosialin | Leiomodin-1 |
| T-cell surface glycoprotein CD8 alpha chain | Lactoperoxidase |
| Complement component C1q receptor | Latent-transforming growth factor beta- |
| | binding protein 2 |
| CUB domain-containing protein 1 | Ly6/PLAUR domain-containing protein 3 |
| Cadherin-2 | Apical endosomal glycoprotein |
| Cadherin-3 | Matrilin-3 |
| Cadherin-related family member 2 | Meprin A subunit beta |
| Cell adhesion molecule-related/down- | Matrix extracellular phosphoglycoprotein |
| regulated by oncogenes | |
| Cadherin EGF LAG seven-pass G-type | Tyrosine-protein kinase Mer |
| receptor 2 | |
| Complement factor H-related protein 5 | Lactadherin |
| Secretogranin-1 | Promotilin |
| Chitotriosidase-1 | Macrophage metalloelastase |
| Chordin-like protein 1 | Myelin-oligodendrocyte glycoprotein |
| Chordin-like protein 2 | Matrix remodeling-associated protein 8 |
| Cytoskeleton-associated protein 4 | Neurocan core protein |
| C-type lectin domain family 14 member A | Neurofilament light polypeptide |
| Contactin-5 | Nucleoside diphosphate kinase 3 |
| Collagen alpha-1(XV) chain | Neurogenic locus notch homolog protein 3 |
| Collagen alpha-3(VI) chain | N-acetylneuraminate lyase |
| Collagen alpha-1(IX) chain | Neuronal pentraxin-2 |
| Complement receptor type 2 | Neurotrophin-3 |
| Corticoliberin | Neurotrophin-4 |
| Cartilage acidic protein 1 | N-terminal prohormone of brain natriuretic |
| | peptide |
| Beta-crystallin B2 | Odontogenic ameloblast-associated protein |
| Chondroitin sulfate proteoglycan 5 | Glycodelin |
| Cystatin-SN | Inactive serine protease PAMR1 |
| Cystatin-D | phospholipase A2 inhibitor and Ly6/PLAUR |
| | domain-containing protein |
| Collagen triple helix repeat-containing | Polycystin-1 |
| protein 1 | |
| Cathepsin F | Tissue-type plasminogen activator |
| Cathepsin L2 | Podocalyxin-like protein 2 |
| Coxsackievirus and adenovirus receptor | Pro-opiomelanocortin |
| Stromal cell-derived factor 1 | Prolargin |
| C-X-C motif chemokine 14 | Prolactin |
| C-X-C motif chemokine 17 | Prion-like protein doppel |
| C-X-C motif chemokine 9 | Prokineticin-1 |
| NADH-cytochrome b5 reductase 2 | Persephin |
| Cytokine-like protein 1 | Prostaglandin-H2 D-isomerase |
| Discoidin, CUB and LCCL domain-containing | Pleiotrophin |
| protein 2 | |
| Decorin | Receptor-type tyrosine-protein |
| | phosphatase mu |
| Divergent protein kinase domain 2B | Receptor-type tyrosine-protein |
| | phosphatase N2 |
| Dickkopf-related protein 3 | Receptor-type tyrosine-protein |
| | phosphatase R |
| Dickkopf-like protein 1 | Receptor-type tyrosine-protein |
| | phosphatase zeta |
| Protein delta homolog 1 | Renin |
| Dentin matrix acidic phosphoprotein 1 | Proto-oncogene tyrosine-protein kinase |
| | receptor Ret |
| Dipeptidase 2 | Repulsive guidance molecule A |
| Dermatopontin | RGM domain family member B |
| Tumor necrosis factor receptor superfamily | Prorelaxin H2 |
| member 27 | |
| Epididymal secretory protein E3-beta | Roundabout homolog 1 |
| EGF-like repeat and discoidin I-like domain- | Ribonucleoside-diphosphate reductase |
| containing protein 3 | subunit M2 |
| EGF-containing fibulin-like extracellular | Scavenger receptor class F member 2 |
| matrix protein 1 | |
| EF-hand domain-containing protein D1 | Secretogranin-2 |
| Epidermal growth factor receptor | Secretogranin-3 |
| Elastin | Uteroglobin |
| Protein enabled homolog | Protein sidekick-2 |
| Endoglin | Neuronal-specific septin-3 |
| Beta-enolase | Superoxide dismutase [Mn], mitochondrial |
| Ectonucleotide | VPS10 domain-containing receptor SorCS2 |
| pyrophosphatase/phosphodiesterase family | |
| member 2 | |
| Ectonucleotide | Sclerostin |
| pyrophosphatase/phosphodiesterase family | |
| member 5 | |
| Receptor tyrosine-protein kinase erbB-4 | Serine protease inhibitor Kazal-type 1 |
| Fatty acid-binding protein, adipocyte | Spondin-2 |
| Protein FAM3B | Small proline-rich protein 3 |
| Prolyl endopeptidase FAP | Sushi repeat-containing protein SRPX |
| Tumor necrosis factor receptor superfamily | Sushi domain-containing protein 2 |
| member 6 | |
| Tumor necrosis factor ligand superfamily | Sushi domain-containing protein 5 |
| member 6 | |
| Fibulin-2 | Trefoil factor 1 |
| Fc receptor-like protein 2 | Thrombospondin-2 |
| Fibroblast growth factor 5 | Tumor necrosis factor receptor superfamily |
| | member 11B |
| Follitropin subunit beta | Tumor necrosis factor receptor superfamily |
| | member 13B |
| Follistatin-related protein 1 | Tumor necrosis factor ligand superfamily |
| | member 13 |
| Growth arrest-specific protein 6 | Tenascin-X |
| Growth/differentiation factor 15 | Tetraspanin-1 |
| Glial fibrillary acidic protein | WAP four-disulfide core domain protein 2 |
| GDNF family receptor alpha-like | Wnt inhibitory factor 1 |
| Appetite-regulating hormone | Protein Wnt-9a |
| Gastric inhibitory polypeptide | Lymphotactin |

In some embodiments, the present invention provides a method for predicting the presence or absence of at least one disease in a subject, predicting the severity of at least one disease in a subject, predicting the risk of a subject developing at least one disease; and/or predicting the risk of mortality of a subject, wherein the method comprises:
a) measuring, in a biological sample obtained from the subject at a first time point, the presence or amount of each biomarker in a set of biomarkers, wherein the set of biomarkers comprises at least 7 biomarkers selected from Table 1.

In some embodiments, the present invention provides a method for predicting the presence or absence of at least one disease in a subject, predicting the severity of at least one disease in a subject, predicting the risk of a subject developing at least one disease, and/or predicting the risk of mortality of a subject, wherein the method comprises:
a) measuring, in a biological sample obtained from the subject at a first time point, the presence or amount of each biomarker in a set of biomarkers, wherein the set of biomarkers comprises at least 50 biomarkers selected from Table 2.

In some embodiments, the method comprises predicting the risk of developing at least one disease in a subject in a given period, and/or predicting the severity of at least one disease in a subject; and/or predicting the risk of mortality of a subject in a given period. In some embodiments the given period is 1, 2, 3, 4, 5, 10, 15, 20, 25, 30, 40, 50, or 60 years. In some embodiments the given period is the remainder of the subject's life.

In some embodiments, the present invention provides a device for determining the presence or amount of each biomarker in a set of biomarkers;
wherein the device comprises a set of probes for detection of the biomarkers in the set of biomarkers, wherein the set of probes is specific for and capable of recognising the set of biomarkers in a biological sample from a subject; and
wherein the set of biomarkers comprises at least 7, 8, 9, 10, 11, 12, 13, 14, 15, 16, 17, 18, 19, or 20 biomarkers selected from Table 1.

In some embodiments, the present invention provides a device for determining the presence or amount of each biomarker in a set of biomarkers,
wherein the device comprises a set of probes for detection of the biomarkers in the set of biomarkers, wherein the set of probes is specific for and capable of recognising the set of biomarkers in a biological sample from a subject; and
wherein the set of biomarkers comprises at least 50, 75, 100, 125, 150, 175, 200 or 204 biomarkers selected from Table 2.

In some embodiments, the present invention provides a set of probes for determining the presence or amount of a set of biomarkers,
wherein each probe in the set of probes specifically recognises at least one biomarker in the set of biomarkers; and
wherein the set of biomarkers comprises at least 7, 8, 9, 10, 11, 12, 13, 14, 15, 16, 17, 18, 19, or 20 biomarkers selected from Table 1.

In some embodiments, the present invention provides a set of probes for determining the presence or amount of a set of biomarkers,
wherein each probe in the set of probes specifically recognises at least one biomarker in the set of biomarkers; and
wherein the set of biomarkers comprises at least 50, 75, 100, 125, 150, 175, 200 or 204 biomarkers selected from Table 2.

In some embodiments, the present invention provides a biomarker testing kit comprising a set of probes as disclosed herein. Suitably, the testing kit may be for use at home or in a point of care setting, and may comprise a suitable sampling device such as a finger prick blood sampling device or a patch based blood sampling device.

In some embodiments, the present invention provides for the use of the device as disclosed herein, the probes as disclosed herein or the biomarker testing kit as disclosed herein; in a method as disclosed herein.

In some embodiments, the present invention provides for a computer-implemented method for determining, predicting or estimating the biological age of a subject comprising the steps of:
a) Obtaining data of the measured levels of: i) at least 7 biomarkers in Table 1; or ii) at least 50 biomarkers in Table 2;
b) Inputting the measured levels in step a) to a predictive model which relates the measured levels with biological age or chronological age; and
c) Outputting a determined, predicted or estimated biological age.

In some embodiments, the present invention provides for a computer-implemented method for predicting the presence or absence of at least one disease in a subject, predicting the risk of a subject developing at least one disease, and/or predicting the risk of mortality of a subject, wherein the method comprises:
a) Obtaining data of the measured levels of: i) at least 7 biomarkers in Table 1; or ii) at least 50 biomarkers in Table 2;
b) Inputting the measured levels in step a) to a predictive model which relates the measured levels with disease and/or mortality; and
c) Outputting at least one of:
   i) the presence or absence of at least one disease in the subject;
   ii) the severity of at least one disease in a subject;
   iii) the risk of the subject developing at least one disease; and/or
   iv) the risk of mortality of the subject.

In some embodiments, the present invention provides for a computer-readable storage medium or a computer program comprising computer-executable instructions, which when executed by a computing system, are capable of causing the computing system to perform any of the methods disclosed herein.

In some embodiments, the set of biomarkers consists of, comprises at least or comprises no more than 8, 9, 10, 11, 12, 13, 14, 15, 16, 17, 18, 19, or 20 biomarkers selected from the biomarkers of Table 1.

In some embodiments the set of biomarkers comprises at least 75, 100, 125, 150, 175, 200 or 204 biomarkers selected from the biomarkers of Table 2.

In some embodiments, the set of biomarkers consists of, comprises at least or comprises no more than 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203 or 204 biomarkers selected from the biomarkers of Table 2.

In some embodiments the set of biomarkers consists of, comprises at least or comprises no more than 7, 8, 9 or 10 biomarkers selected from the biomarkers of Table 3:

TABLE 3

| | |
|---|---|
| Tumor necrosis factor receptor | Elastin |
| superfamily member 27 | |
| Collagen alpha-3(VI) chain | Immunoglobulin superfamily DCC |
| | subclass member 4 |
| Growth/differentiation factor 15 | Follitropin subunit beta |
| Neurofilament light polypeptide | Latent-transforming growth factor |
| | beta-binding protein 2 |
| Podocalyxin-like protein 2 | Prostate-specific antigen |

In some embodiments the biomarkers are selected from polypeptides, polynucleotides, and other body metabolites. A polypeptide may be a protein or fragments of a polypeptide or protein. A polynucleotide may be a DNA or RNA, including siRNA, tRNA, rRNA, and mRNA. In some embodiments the biomarkers are proteins or fragments thereof.

In the aspects of the invention as described herein, the biomarkers are proteins. The invention may measure the presence or amount of each protein in a set of proteins.

In some embodiments the subject is a human or an animal. In some embodiments the subject is a human.

In some embodiments the biological sample is a blood based sample. In some embodiments the blood based sample is plasma or serum.

In some embodiments a method of the invention further comprises
b) measuring, in a further biological sample obtained from the subject at a different time point from step a), the presence or amount of each biomarker in the set of biomarkers;
c) determining the difference in the presence or amount of each biomarker in the set of biomarkers between the measurements of step a) and step b).

Suitably, the set of biomarkers is the same as the set of biomarkers is the same as the set of biomarkers of step a). In an embodiment, the set of biomarkers may be different to the set of biomarkers of step a). In an embodiment, the set of biomarkers used in step b) may include the set of biomarkers used in step a).

In some embodiments a method of the invention further comprises;
d) comparing the measurement of step a), or the determined difference of step c) with a reference measurement obtained from a subject of a known chronological age to determine, predict or estimate a biological age of the subject.

In some embodiments the method further comprises;
e) determining the difference between the chronological age and the biological age of the subject to determine or estimate a value of accelerated or decelerated aging of the subject.

In some embodiments the method further comprises;
e) determining the relationship between the chronological age and the biological age of the subject to determine or estimate a value of an age gap or accelerated/decelerated aging of the subject.

A difference in age refers to one age value being numerically higher or lower than the other age value. A greater age has a numerically higher value than the other age. A lower age has a numerically lower value than the other age with which it is being compared.

In some embodiments a greater chronological age than biological age in the subject indicates decelerated aging of the subject. In some embodiments a greater chronological age than biological age in the subject indicates a negative age gap.

In some embodiments a greater biological age than chronological age in the subject indicates accelerated aging of the subject. In some embodiments a greater biological age than chronological age in the subject indicates a positive age gap.

In some embodiments the method further comprises;
f) using the value of accelerated or decelerated aging or the value of an age gap of the subject to predict:
   i) the presence or absence of at least one disease in the subject;
   ii) the severity of at least one disease in a subject;
   iii) the risk of the subject of having or developing to at least one disease; and/or
   iv) the risk of mortality of the subject.

In some embodiments the method further comprises:
g) comparing the measurement of step a), or the determined difference of step c) with reference measurements from a subject with a known disease, known risk of developing a disease, or known risk or mortality to predict;
   i) the presence or absence of at least one disease in the subject;
   ii) the severity of at least one disease in a subject
   iii) the risk of the subject of having or developing to at least one disease; and/or
   iv) the risk of mortality of the subject.

In some embodiments at least one disease is an age-related disease.

In some embodiments the at least one disease is selected from chronic liver disease, type II diabetes, Parkinson's disease, rheumatoid arthritis, osteoarthritis, macular degeneration, ischemic heart disease, stroke, osteoporosis, ischemic stroke, emphysema, chronic obstructive pulmonary disease (COPD), chronic kidney diseases, all-cause dementia, Alzheimer's disease, oesophageal cancer, prostate cancer, lung cancer, non-Hodgkin lymphoma or combinations thereof.

In some embodiments mortality is selected from all-cause mortality; age-related mortality; or mortality related to; chronic liver disease, type II diabetes, Parkinson's disease, rheumatoid arthritis, osteoarthritis, macular degeneration, ischemic heart disease, stroke, osteoporosis, ischemic stroke, emphysema, chronic obstructive pulmonary disease (COPD), chronic kidney diseases, all-cause dementia, Alzheimer's disease, oesophageal cancer, prostate cancer, lung cancer, non-Hodgkin lymphoma or combinations thereof.

In some embodiments the method is an in vitro and/or ex vivo method.

In some embodiments each probe is independently selected from an antibody, antibody fragment, oligonucleotide, protein, biotin-binding protein, enzyme, fluorophore or combinations thereof.

In some embodiments each probe in the set is independently selected from an antibody, antibody fragment, oligonucleotide, protein, biotin-binding protein, enzyme, fluorophore or combination thereof.

### BRIEF DESCRIPTION OF THE DRAWINGS

FIG. 1. Overview of the study design and analytic approaches. a) UK Biobank (UKB) participants were both split into 70/30 training/test sets. Training the proteomic age clock model was conducted in the UKB training data and performance of the model was tested in the test set. b) Independent data from the China Kadoorie Biobank (CKB) and FinnGen were used for further independent validation of the proteomic age clock model. c) Protein predicted age (ProtAge) was calculated in the full UKB sample using 5-fold cross-validation, with proteomic age acceleration (ProtAgeAccel) calculated as the difference between ProtAge and chronological age. ProtAgeAccel was tested in relation to a comprehensive panel of biological aging markers and measure of frailty and physical/cognitive decline, as well as mortality, 14 common diseases, and 12 common cancers. Most association analyses were carried out in the UKB only, due to smaller sample in the CKB and lack of disease cases in FinnGen.

FIG. 2. Baseline characteristics and proteomic aging clock performance across cohorts. a) Density plot of age at recruitment in the UK Biobank (UKB), China Kadoorie Biobank (CKB), and FinnGen. b) Density plot of age at death in the UKB (10.6%) and CKB (9%)—FinnGen only had 1.1% mortality. c) Counts of prevalent and incident cases of all common diseases studied in the UKB sample (n=45,441). d) Performance of the trained proteomic aging model in the UKB holdout test set (n=13,633). e) Performance of the trained proteomic aging model in the CKB (n=3,977). f) Performance of the trained proteomic aging model in FinnGen (n=1,990). g) Sex specific distributions of ProtAgeAccel in the UKB, CKB, and FinnGen. h) Distributions of ProtAgeAccel according to self-reported ethnicity in the UKB. i) Distributions of ProtAgeAccel according to geographic region of residence in the CKB. Correlation coefficients shown in d-f are Pearson correlation coefficients. Violin plots in g-i show both the median (white dot) and interquartile range. COPD: chronic obstructive pulmonary disease, ProtAge: protein predicted age, ProtAgeAccel: proteomic age acceleration (in years).

FIG. 3. ProtAgeAccel is associated with age-related biological, physical, and cognitive status. a) Associations between ProtAgeAccel and biological aging mechanisms in the full UKB sample (n=45,441). b) Associations between ProtAgeAccel and measures of physiological and cognitive (reaction time, fluid intelligence) status in the full UKB sample (n=45,441). c) Associations between ProtAgeAccel and biological aging mechanisms in the subsample of UKB participants with no lifetime diagnosis of any of the 26 diseases studied (n=20,353). d) Associations between ProtAgeAccel and measures of physiological and cognitive status in the subsample of UKB participants with no lifetime diagnosis of any of the 26 diseases studied (n=20,353). All models used linear or logistic regression and were adjusted for age, sex, Townsend deprivation index, recruitment centre, ethnicity, IPAQ activity group, and smoking status. Estimates in dark circles are from the full 204-protein model, whereas estimates in light diamonds are from the smaller proteomic age clock model with 20 proteins (ProtAgeAccel20). ALT: alanine aminotransferase, AST: aspartate aminotransferase, BMI: body mass index, FEV1: forced expiratory volume in 1 second, GGT: Gamma-glutamyl Transferase, IGF-1: insulin-like growth factor 1, ProtAgeAccel: proteomic age acceleration (in years).

FIG. 4. ProtAgeAccel predicts age-specific mortality and disease risk trajectories in the UKB and CKB. Cumulative incidence plots for the top, median, and bottom deciles of ProtAgeAccel in a) UK Biobank (UKB; total random participants n=45,441) and b) China Kadoorie Biobank (CKB; n=3,977). Number of incident cases are shown for each disease—these numbers reflect the total number of incident cases present only among those in the 3 deciles shown, not the full dataset. Incidence rates are shown for the subsequent 11-16 years (UKB) or 11-14 years (CKB) of follow-up after recruitment for each given age at recruitment (e.g., the cumulative incidence rate shown at age 65 in a) is the rate of incident cases in the 11-16 years of follow up for those aged 65 at recruitment). All plots show 95% confidence intervals in lighter shading. Diseases shown here for the CKB are those with greater than 50 cases across the three deciles of ProtAgeAccel. ProtAgeAccel: proteomic age acceleration (in years).

FIG. 5. Effect size of ProtAgeAccel on mortality and common diseases are largely invariant to covariate adjustment. Associations between ProtAgeAccel and mortality or diseases in Cox proportional hazards models with increasing levels of covariate adjustment. All models were run in the UK Biobank (UKB; n=45,441). a). Model 1 is adjusted for age and sex. b) Model 2 is adjusted for age, sex, ethnicity, Townsend deprivation index, recruitment centre, IPAQ activity group, and smoking status. c) Model 3 is adjusted for age, sex, ethnicity, Townsend deprivation index, recruitment centre, IPAQ activity group, smoking status, BMI, and prevalent hypertension. Estimates in dark circles are from the full 204-protein model, whereas estimates in light diamonds are from the smaller proteomic age clock model with 20 proteins (ProtAgeAccel20). ProtAgeAccel: proteomic age acceleration (in years).

FIG. 6. Stability of ProtAge protein associations with age across 3 time points. Comparison of betas for the association between age and each of the 149 ProtAge APs with repeat measurements available during baseline and two follow up imaging visits (n=1,085). a) Comparison of betas for the association between age and each of the 149 ProtAge APs during baseline and the 2014+ follow up imaging visit. b) Comparison of betas for the association between each of these 149 ProtAge APs and age during baseline and the 2019+ imaging visit. c) Comparison of betas for the association between each of the 149 ProtAge APs and age during the 2014+ imaging visit and during the 2019+ imaging visit. Shown in each plot are the Pearson correlation coefficient (r), p-value for the correlation, and the model slope (A). APs: aging-related proteins.

FIG. 7. Associations between ProtAgeAccel and 12 common cancers in the UKB. Associations between ProtAgeAccel and incident cancer diagnosis in Cox proportional hazards models with increasing levels of covariate adjustment. All models were run in the UK Biobank (UKB; n=45,441). a). Model 1 is adjusted for age and sex. b) Model 2 is adjusted for age, sex, Townsend deprivation index, recruitment centre, IPAQ activity group, and smoking status. c) Model 3 is adjusted for age, sex, Townsend deprivation index, recruitment centre, IPAQ activity group, smoking status, BMI, and prevalent hypertension. ProtAgeAccel: proteomic age acceleration (in years).

FIG. 8. Effect size of ProtAgeAccel on mortality and disease among non-smokers and those within normal weight range. Associations between ProtAgeAccel and mortality or diseases among UK Biobank participants who report being never smokers (n=24,528) (a) and with a BMI≥18.5 and <25 kg/m2 (n=14,555) (b). All models are Cox proportional hazards models using model 2 (adjusted for age, sex, Townsend deprivation index, recruitment centre, and IPAQ activity group). ProtAgeAccel: proteomic age acceleration (in years).

FIG. 9. ProtAgeAccel increases linearly with increasing disease multimorbidity. a) Average years of ProtAgeAccel in those with 1 disease diagnosis or 2, 3, 4+ comorbid conditions compared with average ProtAgeAccel in those with no diagnoses among UK Biobank (UKB) participants 40-50 years old at recruitment. b) Average years of ProtAgeAccel in UKB participants with 1 disease diagnosis or 2, 3, 4+ comorbid conditions compared with average ProtAgeAccel in those with no diagnoses aged 51-65 years old at recruitment. c) Percentages of the UKB population with 0, 1, 2, 3, and 4+ lifetime disease diagnoses. d) Average years of ProtAgeAccel according to levels of self-rated health in the UKB. In a) and b), values on the y-axis represent the average years of ProtAgeAccel for each group compared with the average in those with no diagnoses (calculated as the difference in average ProtAgeAccel between the two groups). Multimorbidity is defined as the number of lifetime diagnoses of any of the 26 diseases analyzed in this study. In a, b, and d, error bars are shown as the standard error of the mean. ProtAgeAccel: proteomic age acceleration (in years).

FIG. 10. PPI network of ProtAge APs from the STRING database. Protein-protein interaction (PPI) network of a highly interconnected subset of APs in the ProtAge model with at least 2 node connections using experimental PPI information from the STRING database. Proteins are sized and colored by number of connections, with those showing a greater number of connections with other proteins displayed larger and lighter color.

FIG. 11. PPI network of ProtAge APs using SHAP values. Protein-protein interaction (PPI) network using SHAP values from the trained model. Proteins shown are only those that are highly interconnected using a cutoff of 0.0083 for absolute SHAP interaction values. Proteins are sized and colored by number of connections, with those showing a greater number of connections with other proteins displayed larger and lighter color.

FIG. 12. Model benchmarking for estimation of proteomic age in the UK Biobank and China Kadoorie Biobank. Scatterplots comparing actual chronological age (x-axis) versus protein predicted age (protAge; y-axis) in a) the UK Biobank test set (n=13,633); b) China Kadoorie Biobank (n=3,977); and c) FinnGen (n=1,990). Models compared included two penalized linear regression models (LASSO, elastic net), one gradient boosting machine learning model (LightGBM), and three neural network architectures (ResNet, MLP, TabR). LASSO: least absolute shrinkage and selection operator; MAE: mean absolute error; MLP: multilayer perceptron; RMSE: root mean square error.

FIG. 13. Performance of proteomic age clocks with decreasing numbers of proteins in the UKB. Plots shown are the comparison of actual chronological age versus protein predicted age from three LightGBM models using: a) all 2,987 proteins considered, b) 204 proteins identified in the Boruta feature selection process, c) 20 proteins identified through further recursive feature elimination analysis using SHAP values. d) Models were tested iteratively using 5-fold cross-validation starting from 204 proteins down to 5 proteins. At each step, the protein with the smallest absolute mean SHAP values across the folds was discarded. For each model, the R² of explained variance in chronological age is presented as the average R² across all 5 folds. Correlation coefficients (r) shown are from a Pearson correlation test. MAE: mean absolute error; ProtAge: protein predicted age; RMSE: root mean square error.

FIG. 14. Proteomic age model performance across age bins in the UKB test set. The performance of the 2,897-protein model is shown in the full UKB test set (a), as well as in the subset of participants aged 40-50 years (b), 50-60 years (c), and 60-70 years (d). MAE: mean absolute error; RMSE: root mean square error; UKB: UK Biobank.

FIG. 15. Proteomic age estimation accuracy by sex in the UKB. Comparison of actual chronological age versus protein predicted age (ProtAge) for a model using: a) all participants; b) female participants only; c) male participants only; Model accuracy metrics comparing predicted versus actual age values are shown as Pearson r correlation coefficient, R², root mean square error (RMSE), and mean absolute error (MAE). d) Comparison of protein predicted age (ProtAge) for the same female participants from the all participant model (y-axis) and model with only female participants (x-axis). e) Comparison of protein predicted age (ProtAge) for the same male participants from the all participant model (y-axis) and model with only male participants (x-axis). In both d and e, the Pearson r correlation coefficient, p-value for correlation and slope of the best fit line (A) are shown for comparison of the two predicted ages.

### DEFINITIONS

Herein, a “biomarker” is a molecule that is associated either quantitatively or qualitatively with a biological change. A “biomarker” may be a compound that is differentially present (i.e., increased or decreased) in a biological sample from a subject or a group of subjects having a first phenotype (e.g., having a biological age, or disease or condition) as compared to a biological sample from a subject or group of subjects having a second phenotype (e.g., not having the said biological age, disease or condition or having a less severe version of the disease or condition).

A “protein” (used interchangeably with the terms “polypeptide,” and “peptide”) is a polymer of at least two amino acids covalently linked by an amide bond. A protein may be any suitable length, and may comprise post-translational modification, for example glycosylation, phosphorylation, lipidation, myristilation, ubiquitination, etc. A protein may comprise D- and L-amino acids, and mixtures of D- and L-amino acids.

As used herein, “omics” refers to any of several areas of biological study defined by the investigation of the entire complement of a specific type of biomolecule or the totality of a molecular process within an organism. In biology the word “omics” refers to the sum of constituents within a cell. The omics sciences share the overarching aim of identifying, describing, and quantifying the biomolecules and molecular processes that contribute to the form and function of cells and tissues.

Therefore, by the term “ome” or “omic” or “omic data” refers to data generated from the study of one or more of the “omes” of an organism, for example the genome (all the genetic material), proteome (all the protein and peptide material), transcriptome (all of the RNA molecules), metabolome (all of the small molecules), interactome (all of the interactions, for example protein-protein, nucleic acid-protein), epigenome (all of the alterations other than the DNA sequence that may change gene activity such as changes in DNA methylation [CpG methylation], chromatin accessibility, histone modifications, among others), microbiome (collection of all the microorganisms and viruses that live in a given environment, including the human body or part of the body, such as the digestive system) etc.

As used herein, the term “proteomic” refers to the large-scale study of proteins or proteome. A “proteome” is the entire complement of proteins produced in an organism, system, or biological context. A proteome may refer to the proteome of a species (for example, Homo sapiens) or an organ (for example, the liver) or any biological sample (for example, a blood-based sample), for example as defined herein. The proteome is not constant; it differs from cell to cell and changes over time. To some degree, the proteome reflects the underlying genome and transcriptome. However, protein activity (often assessed by the reaction rate of the processes in which the protein is involved) is also modulated by many factors in addition to the expression level of the relevant gene. Herein the proteome refers to the entire set of proteins of a biological sample.

The terms “polynucleotide,” “oligonucleotide,” “nucleic acid” and “nucleic acid molecule” are used herein to refer to a polymeric form of a nucleotide of any length, either ribonucleotides or deoxyribonucleotides. This term refers only to the primary structure of the molecule. There is no intended distinction in length between the terms “polynucleotide,” “oligonucleotide,” “nucleic acid” and “nucleic acid molecule,” and these terms are used interchangeably.

A “genome” is the entire complement of genetic material of an organism, system, or biological context. A genome may include coding and non-coding sequences. A genome refers to all DNA sequences. Where the term genome is used to refer to DNA sequences, the term “transcriptome” may be used to refer to the RNA material of the organism, system, or biological context. A genome, epigenome, or transcriptome may refer to that of a species (for example, Homo sapiens) or an organ (for example, the liver), or any biological sample (e.g. a blood-based sample), for example as defined herein. The genome is constant; however the epigenome and transcriptome may differ from cell to cell and change over time.

A “fragment” refers to a part of a whole biological molecule, for example a protein, nucleic acid, or antibody. A fragment may comprise at least 70%, 80%, 90%, 95%, 98%, and 99% of the full-length molecule.

A “biological sample” refers to any type of biological material derived from a living organism. A blood-based sample refers to any type of biological material derived from the blood of a living organism.

A “reference” as used herein is an item which is used for comparison purposes. For example, a reference may be a value of chronological age or may be a biomarker level, amount, concentration, or profile which is used for comparison purposes against the measure obtained in a method of the invention. A reference may be from the same or a different subject to which the invention is applied. A reference may be a predetermined threshold value.

As used herein, the terms “biological age”, “physiological age” and “proteomic age” are used synonymously. As used herein, biological age, physiological age and proteomic age refer to an estimation of age using ‘omics data or biomarker data to capture the level of biological functioning of an individual in association with an expected level of functioning for a given chronological age.

As used herein “in-vitro” refers to methods that are performed with microorganisms, cells, or biological materials outside their normal biological context. Typically, these methods are performed in labware such as test tubes, flasks, Petri dishes, and microtiter plates. Sometimes in-vitro methods use components of an organism that have been isolated from their usual biological surroundings to permit a more detailed or more convenient analysis than can be done with whole organisms. Herein, in vitro refers to a method which is performed on a sample which has been obtained from a subject.

As used herein “ex-vivo” refers to experimentation or measurements done in or on tissue from an organism in an external environment with minimal alteration of natural conditions. For example, the measurements can be performed on an isolated tissue or organ from the subject such as the blood, liver, heart, spleen, muscle, tumour sample, blood vessel or combinations thereof.

As used herein “prediction” refers to a method of assigning a probability or likelihood for when or where an event is likely to occur based upon specific data sources.

As used herein “estimation” refers to a value that is usable for some purpose even if input data may be incomplete, uncertain, or unstable. The value is nonetheless usable because it is derived from the best information available. Typically, estimation involves using the value of a statistic derived from a sample to estimate the value of a corresponding population parameter. The sample provides information that can be projected, through various formal or informal processes, to determine a range most likely to describe the missing information.

A “biological age clock” refers to an estimate of biological age. It represents any biological system or biomarker that changes during age. Measuring the amount of variation in those biological systems or biomarkers can allow the determination of how far an organism has drifted from youthful function or how close they are to morbidity and mortality. Biological age clocks specifically aim to determine a biological age of a subject.

“Chronological age” refers to the number of days, weeks, months and/or years that have elapsed since a subject's birth.

As used herein “disease” refers to any disorder of structure or function in a human, animal, or plant.

As used herein “mortality” refers to the action or fact of dying and/or the cessation of life of an organism.

As used here “predetermined threshold value” refers to the level or amount of at least one of the plurality of biomarkers above or below. The predetermined threshold values indicates a point at which the subject likely has a particular biological age, a particular risk of having or developing at least one disease; and/or a particular risk of mortality.

As used herein, “a measurement for use in determining, predicting or estimating the biological age of a subject” is any quantitative value or any qualitative value. Said values can be further processed to usefully aid the user of the invention in determining, predicting or estimating the biological age of a subject.

As used herein the term “risk of mortality” refers to a value determined by calculating a relationship between the presence or amount of the biomarkers in the set of biomarkers in a reference measurement from a subject having a known risk of mortality/death and the presence or amount of the biomarkers in the set of biomarkers in subjects with an unknown risk of mortality. Alternatively the term risk of mortality refers to a value determined by correlation of the presence or amount of the biomarkers in the set of biomarkers in a reference measurement from a subject having a known Acute Physiology and Chronic Health Evaluation (APACHE I to IV) (Zimmerman et al. 2006) and/or Pediatric Risk of Mortality (PRISM) (Pollack et al. 2015) score against the presence or amount of the biomarkers in the set of biomarkers in subjects with an unknown risk of mortality. The risk of mortality can be any of the risk of mortalities disclosed herein. “Risk of mortality” can also refer to the probability or likelihood of the subject dying in a given period of time. In some embodiments, the invention measures the presence or amount of each protein in a set of proteins.

As used herein, the term disease risk refers to the probability or likelihood of the subject developing a disease, or a particular severity of a disease, in a given period of time. In some embodiments, mortality or disease risk can be determined by analyzing the presence or amount of the biomarkers in the set of biomarkers. In some embodiments, mortality or disease risk can be determined by using the age gap or accelerated/decelerated aging value. The presence or absence of the biomarkers in the set of biomarkers or particular amounts of the biomarkers of the set of biomarkers of the disclosure as described herein can be characteristic of mortality or disease risk. Risk can encompass both increased or decreased risk. The disease can be any of the diseases disclosed herein. In some embodiments, the invention measures the presence or amount of each protein in a set of proteins.

As used herein, risk of developing a disease can refer to a likelihood of a subject towards the development of a disease, or towards being less able to resist a particular disease than one or more reference subjects. Risk of developing a disease also refers to the future risk of a subject developing at least one disease within a defined time period in the future. In some embodiments the defined time period is 1, 2, 3, 4, 5, 10, 15, 20, 25, 30, 40, 50, or 60 years. The future risk may be relative to a reference subject having the same chronological age (measured in years) as the subject in question. For example, an increased risk of developing a disease can be indicative of an increased likelihood of developing at least one disease compared to a similarly aged reference subject and a decrease risk of disease can be indicative of a decreased likelihood of developing at least one disease compared to a similarly aged reference subject. Risk of disease can encompass increased risk of disease. For example, the presence or absence of the biomarkers in the set of biomarkers or particular amounts of the biomarkers of the set of biomarkers of the disclosure as described herein can be characteristic of increased risk of development of a disease. Risk of disease can encompass decreased risk of disease. For example, the presence or absence of the biomarkers in the set of biomarkers or particular amounts of the proteins of the set of proteins of the disclosure as described herein can be characteristic of decreased risk of development of a disease. The disease can be any of the diseases disclosed herein.

As used herein, a severity of disease refers to the extent of organ system derangement or physiologic decompensation for a subject. A severity of disease in a subject may be minor, moderate, major, or extreme severity. In certain embodiments, severity may be defined by a known clinical, biological, or medical disease severity rating system. Such rating systems are known in the art.

As used herein, positive age gap or accelerated aging is indicated when the biological age of a subject is greater than the chronological age of a subject. Positive age gap and accelerated aging are used synonymously.

As used herein, negative age gap or decelerated aging is indicated when the biological age of a subject is less than the chronological age of a subject. Negative age gap and decelerated aging are used synonymously.

Difference as determined in step (e), age gap or accelerated/decelerated aging can be determined by subtracting the chronological age from the biological age of a subject. Alternatively, age gap or accelerated/decelerated aging can be estimated by determining the relationship between the biological and chronological age of the subject through regression or other statistical methods and extracting information from this model to estimate an age gap or measure of accelerated/decelerated aging. Information extracted can be residuals or other metrics resulting from the statistical method used. These techniques are well known in the art (Rutledge et al. 2022).

As used herein, the term “probe” is used synonymously with “molecular probe” and refers to a group of atoms or molecules used in molecular biology or chemistry to study the properties of other molecules or structures. If some measurable property of the molecular probe used changes when it interacts with the analyte (such as a change in absorbance), the interactions between the probe and the analyte can be studied. Antibodies can be probes. Radioactive isotopes, enzymes and fluorescent dyes are different types of chemical tags that can been used to make probes detectable.

An “antibody” is used in reference to any immunoglobulin molecule that reacts with a specific antigen. An immunoglobulin can derive from any of the commonly known isotypes, including but not limited to IgA, secretory IgA, IgG and IgM. IgG subclasses are also well known to those in the art and include but are not limited to human IgGI, IgG2, IgG3 and IgG4. “Isotype” refers to the antibody class or subclass (e.g., IgM or IgGI) that is encoded by the heavy chain constant region genes.

The phrase “specifically binds to and recognises” or “specifically recognises” with reference to binding of a probe to a biomarker (for example an antibody to an antigen such as a protein in a set of proteins) refers to a binding reaction that is determinative of the presence of the antigen in a heterogeneous population of proteins and other biologies. Thus, under designated immunoassay conditions, the specified antibodies bind to a particular antigen at least two times over the background and do not substantially bind in a significant amount to other antigens present in the sample. Specific binding to an antigen under such conditions may require an antibody that is selected for its specificity for a particular antigen. For example, antibodies raised to an antigen from specific species such as rat, mouse, or human can be selected to obtain only those antibodies that are specifically immunoreactive with the antigen and not with other proteins, except for polymorphic variants and alleles. This selection may be achieved by subtracting out antibodies that cross-react with molecules from other species. A variety of immunoassay formats may be used to select antibodies specifically immunoreactive with a particular antigen. For example, solid-phase ELISA immunoassays are routinely used to select antibodies specifically immunoreactive with a protein (see, e.g., Harlow & Lane. Antibodies, A Laboratory Manual (1988), for a description of immunoassay formats and conditions that can be used to determine specific immunoreactivity). Typically, a specific or selective reaction will be at least twice background signal or noise and more typically more than 10 to 100 times background.

A “set of biomarkers” is plurality of biomarkers, suitably two or more predetermined biomarkers. The set can include at least 7, 8, 9, 10, 11, 12, 13, 14, 15, 16, 17, 18, 19, or 20 of the biomarkers selected from Table 1; at least 50, 75, 100, 125, 150, 175, 200 or 204 of the biomarkers selected from Table 2; or at least 7, 8, 9 or 10 of the biomarkers selected from Table 3.

The present invention can measure the presence or absence of a biomarker in a sample, and/or the amount of a biomarker in a sample. As used herein, “presence” of a biomarker is defined by a measurement signal at or above the limit of detection of the detection method being used. As used herein, “absence” of a biomarker is defined by a measurement signal below the limit of detection of the detection method being used. As used herein, “amount” of a biomarker is defined as an absolute or relative concertation or expression level.

The terms “determining”, “measuring”, “evaluating”, “assessing,” “assaying,” and “analyzing” are used interchangeably herein to refer to any form of measurement, and include determining if an element is present or not. These terms include both quantitative and/or qualitative determinations. Assaying may be relative or absolute. For example, “measuring” can be determining whether the expression level is “less than” or “greater than” or “equal to” a particular threshold, (the threshold can be pre-determined or can be determined by measuring a control sample). On the other hand, “measuring the presence or amount of each biomarker in a set of biomarkers” can mean determining a quantitative value (using any convenient metric) that represents the level of expression (i.e., expression level, e.g., the amount of protein and/or RNA, e.g., mRNA) of a particular biomarker. The level of expression can be expressed in arbitrary units associated with a particular assay (e.g., fluorescence units, e.g., mean fluorescence intensity (MFI)), or can be expressed as an absolute value with defined units (e.g., number of mRNA transcripts, number of protein molecules, concentration of protein, etc.). Additionally, the level of expression of a biomarker can be compared to the expression level of one or more additional biomarkers (e.g., nucleic acids and/or their encoded proteins) to derive a relative or normalized value that represents a normalized expression level. The specific metric (or units) chosen is not crucial as long as the same units are used (or conversion to the same units is performed) when biological samples from the same individual (e.g., biological samples taken at different points in time from the same individual). This is because the units cancel when calculating a fold-change (i.e., determining a ratio) in the expression level from one biological sample to the next (e.g., biological samples taken at different points in time from the same individual).

The term “model” refers to any computational model that may be used to perform the analyses described herein. The model may be a trained or untrained model. Where the model is an untrained model, the predictive model compares the measured levels with a reference measurement obtained from a subject of a known chronological age.

The model may be a machine learning model. For example the model may be a LASSO or elastic net model, a neural network, a large language model, a gradient boosting model (e.g., LightGBM, XGBoost), a support vector machine model, or a tree-based model (e.g., random forest).

Unless defined otherwise, all technical and scientific terms used herein have the same meaning as commonly understood by one of ordinary skill in the art to which this disclosure belongs. For example, the Concise Dictionary of Biomedicine and Molecular Biology, Juo, Pei-Show, 2nd ed., 2002, CRC Press; The Dictionary of Cell and Molecular Biology, 3rd ed., Academic Press; and the Oxford University Press, provide a person skilled in the art with a general dictionary of many of the terms used in this disclosure.

### DETAILED DESCRIPTION

The present invention is based upon the identification of a number of biomarkers that can be used to determine or estimate biological aging or disease status in a subject. This provides a biologically and medically useful measure of biological aging or disease status.

It has been further established by the inventors that a specific subset of the biomarkers can also be used to predict biological aging and/or disease status in a subject. Reducing the number of biomarkers allows for easier and more convenient measurements and therefore improves the usability of the panel.

Each set of biomarkers has also been validated across diverse populations and is predictive of aging and disease.

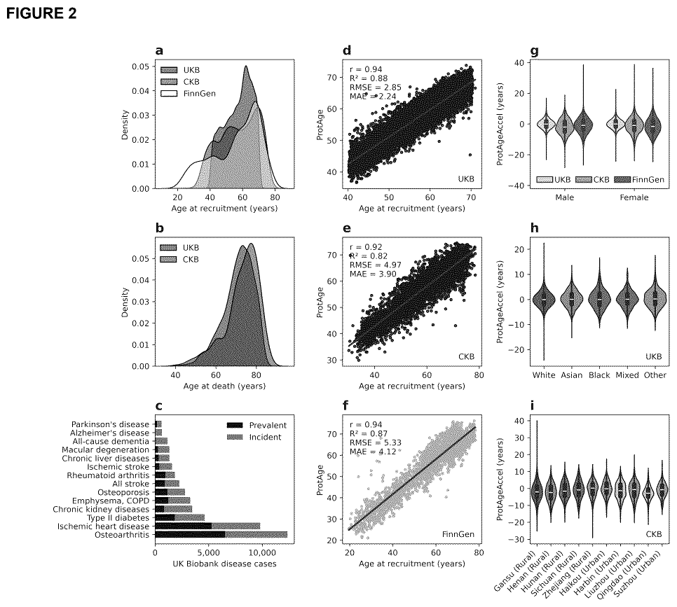

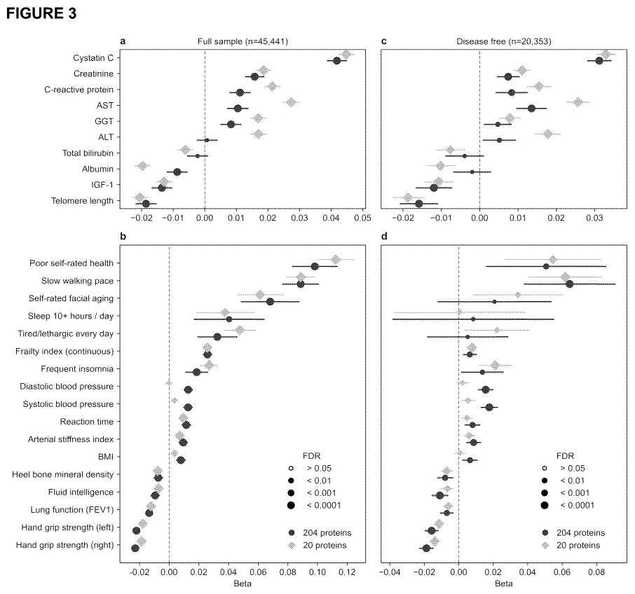

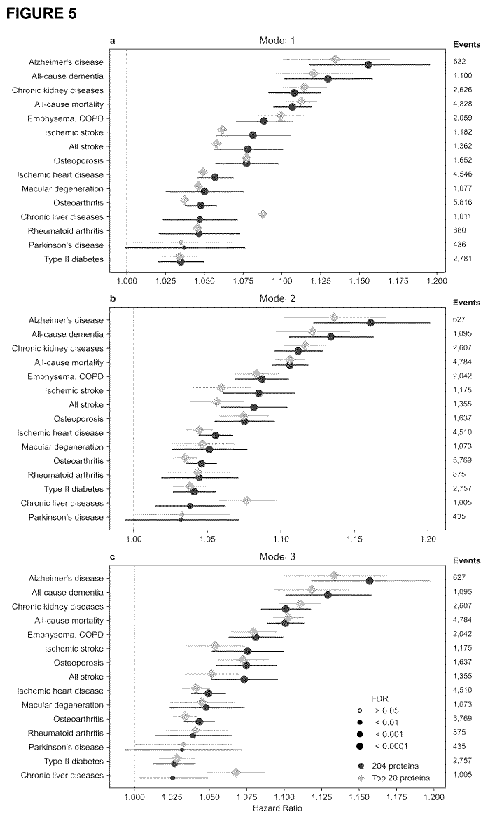

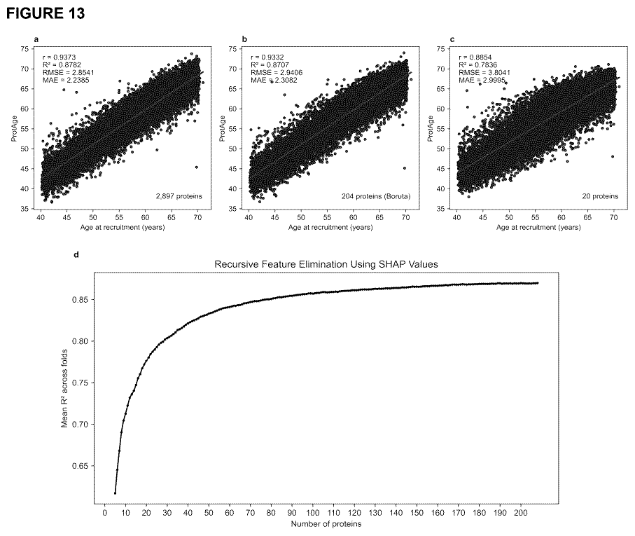

The inventors have developed a proteomic age clock in the UK Biobank (n=45,441). The inventors have shown that using proteomic data generated from the Olink Explore 3072 panel, they can predict a participant's biological age with very high accuracy using all 2,897 proteins on the panel (FIG. 13a), and even in much smaller sets of 204 proteins (FIG. 13b) or 20 proteins (FIG. 13c). The accuracy of these models remains similar when validated in diverse populations from China (n=4,000) and Finland (n=1,990), which indicates that this model generalizes well to other diverse populations (FIG. 2). To date, these models have been validated in participants ranging from 20-90 years of age. The 204-protein model and the 20-protein model are predictive of many chronic diseases and mortality (FIG. 5); as well as predictive of biochemical, functional, and subjective markers of aging (FIG. 3) that the inventors tested in the UK Biobank. The present inventors have surprisingly shown that a single panel of proteins can be used to predict a number of age-related diseases.

The present inventors have also surprisingly shown that the model is transferable between different ethnic and geographic populations. The present inventors surprisingly have shown that a model trained to estimate biological age from proteins in one population (i.e., predominantly white Europeans in the UK Biobank) performs well in other populations that are distinct from the training population in terms of genetic ancestry and geography (FIG. 2).

Further features of certain embodiments of the present invention are described below. The practice of embodiments of the present invention will employ, unless otherwise indicated, conventional techniques of molecular biology, microbiology, recombinant DNA technology and immunology, which are within the skill of those working in the art.

Most general molecular biology, microbiology recombinant DNA technology and immunological techniques can be found in Sambrook et al, Molecular Cloning, A Laboratory Manual (2001) Cold Harbor-Laboratory Press, Cold Spring Harbor, N.Y. or Ausubel et al., Current protocols in molecular biology (1990) John Wiley and Sons, N.Y.

Before the present compositions, methods, and kits are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

The methods of the present invention comprises the step of measuring, in a biological sample obtained from the subject at a first time point, the presence or amount of each biomarker in a set of biomarkers.

A method of the present invention may be practised on a biological sample of any suitable subject, where it is desirable to understand any difference between chronological and biological age in the subject, or where it is desirable to assess the presence, absence or likelihood of a disease in a subject or where it is desirable to assess a risk of mortality in a subject, for example as described herein. A subject may be an animal or a human. The subject may have one or more symptoms of a disease as recited herein. The subject may be suspected of having a disease recited herein. The subject may wish to know their risk of having or dying from a disease recited herein. The subject may wish to know their biological age in comparison to their chronological age. The subject may be a human adult. A human adult may be a human with a chronological age of at least 15, 20, 25, 30, 35, 40, 45, 50, 55, 60, 65, 70, 75, 80, 85, 90, 95, 100, 105, 110, or 115 years, or any integer there between. The subject may be an animal and the method is used for veterinary health purposes. For example, the animal might be a dog, cat, horse, cow, pig, or rabbit. The subject may be an animal and the method may be developed or validated in a laboratory animal. For example, the laboratory animal might be a rodent including mice, rats and hamsters, a primate including chimpanzees, or another model organism used in the art.

Therefore, in a suitable embodiment, there is provided a method for determining, predicting or estimating the biological age of a human adult, or for providing a measurement for use in determining, predicting or estimating the biological age of a subject, wherein the method comprises:
a) measuring, in a biological sample obtained from the subject at a first time point, the presence or amount of each biomarker in a set of biomarkers, wherein the set of biomarkers comprises at least 7, 8, 9, 10, 11, 12, 13, 14, 15, 16, 17, 18, 19, or 20 biomarkers selected from the biomarkers of Table 1 or at least 50, 75, 100, 125, 150, 175, 200 or 204 biomarkers selected from the biomarkers of Table 2.

There is also provided a method for predicting the presence or absence of at least one disease in a human adult, predicting the risk of a subject of having or developing at least one disease; and/or predicting the risk of mortality of a subject, wherein the method comprises:
a) measuring, in a biological sample obtained from the subject at a first time point, the presence or amount of each biomarker in a set of biomarkers, wherein the set of biomarkers comprises at least 7, 8, 9, 10, 11, 12, 13, 14, 15, 16, 17, 18, 19, or 20 biomarkers selected from the biomarkers of Table 1 or at least 50, 75, 100, 125, 150, 175, 200 or 204 biomarkers selected from the biomarkers of Table 2.

Suitably, the biomarkers are proteins and the invention measures the presence or amount of each protein in a set of proteins. Suitably, the subject is a human adult.

In some embodiments the biological sample is a blood-based sample. The sample can be whole blood which is a blood sample that has been collected with an anti-coagulant but is not processed further. The sample can be plasma which is whole blood that is collected in tubes that are treated with an anticoagulant. The blood does not clot in the plasma tube. The cells are pelleted by centrifugation. The supernatant, designated plasma, is removed from the cell pellet. The sample can be serum which is whole blood that is allowed to clot by leaving it undisturbed at room temperature. This takes around 15-30 minutes. The clot is removed by centrifugation. The resulting supernatant, designated serum, is removed from the cell pellet.

In some embodiments, the biological sample can be a cell sample such as a blood sample, a tissue sample, a urine sample, a saliva sample, a semen sample, a faeces or a stool sample, a bone marrow sample, cerebrospinal fluid (CSF), a DNA or RNA sample, a hair sample, a skin sample, a nail sample, an organ, or combinations thereof. For example, a method of the invention can be performed on an isolated tissue or organ from the subject such as the liver, heart, spleen, muscle, tumour sample, blood vessel or combinations thereof. A method of the present invention may comprise processing a biological sample to provide a protein sample thereof.

A biological sample may be obtained from a subject in any suitable manner. A biological sample may be obtained from a subject by a medical practitioner, for example in a point of care location, or may be provided by the subject. A biological sample may be obtained in a separate location to performance of a method of the invention. A biological sample may be processed, such as by centrifugation, filtration, precipitation, dialysis, chromatography, treatment with reagents, washed, or enriched, frozen, defrosted, or fixed, prior to performing a method of the invention. Therefore, a sample as referred to herein may include a biological sample obtained from a subject which has not been processed in any way (a native sample) or may include a processed sample. A sample may be provided in any suitable form, for example processed, extracted, filtered, fractionated, fixed, frozen or defrosted.

It will be understood by one of ordinary skill in the art that in some cases, it is convenient to wait until multiple samples have been obtained prior to assaying the samples. Accordingly, in some cases an isolated biological sample is stored until all appropriate samples have been obtained. One of ordinary skill in the art will understand how to appropriately store a variety of different types of biological sample and any convenient method of storage may be used (e.g., refrigeration) that is appropriate for the particular biological sample. In some embodiments, a biological sample from a first time point is analysed prior to obtaining a biological sample from a second time point. In some cases, a biological sample from a first time point and a biological sample from a second time point are analysed in parallel. In some cases, biological samples are processed immediately or as soon as possible after they are obtained.

The terms “obtained” or “obtaining” as used herein can also include the physical extraction or isolation of a biological sample from a subject. Accordingly, a biological sample can be isolated from a subject (and thus “obtained”) by the same person or same entity that subsequently measures a set of biomarkers in the sample, or by a different person or entity, including the subject themselves. When a biological sample is “extracted” or “isolated” from a first party or entity and then transferred (e.g., delivered, mailed, etc.) to a second party, the sample was “obtained” by the first party (and also “isolated” by the first party), and then subsequently “obtained” (but not “isolated”) by the second party. Accordingly, in some embodiments, the step of obtaining does not comprise the step of isolating a biological sample.

In a suitable embodiment, there is provided a method for determining, predicting or estimating the biological age of a subject, or for providing a measurement for use in determining, predicting or estimating the biological age of a subject, wherein the method comprises:
a) measuring, in a blood, serum or plasma sample obtained from the subject at a first time point, the presence or amount of each biomarker in a set of biomarkers, wherein the set of biomarkers comprises at least 7, 8, 9, 10, 11, 12, 13, 14, 15, 16, 17, 18, 19, or 20 biomarkers selected from the biomarkers of Table 1 or at least 50, 75, 100, 125, 150, 175, 200 or 204 biomarkers selected from the biomarkers of Table 2.

There is also provided a method for predicting the presence or absence of at least one disease in a subject, predicting the risk of a subject of having or developing at least one disease; and/or predicting the risk of mortality of a subject, wherein the method comprises:
a) measuring, in a blood, serum or plasma sample obtained from the subject at a first time point, the presence or amount of each biomarker in a set of biomarkers, wherein the set of biomarkers comprises at least 7, 8, 9, 10, 11, 12, 13, 14, 15, 16, 17, 18, 19, or 20 biomarkers selected from the biomarkers of Table 1 or at least 50, 75, 100, 125, 150, 175, 200 or 204 biomarkers selected from the biomarkers of Table 2.

Examples of suitable biomarkers for use in the present invention include polypeptides, proteins or fragments of a polypeptide or protein; and polynucleotides, such as a gene product, RNA or RNA fragment; and other body metabolites. Suitably, a biomarker is a protein or a fragment thereof. Suitably, a biomarker is a nucleic acid. Suitably, a set of biomarkers may comprise a combination of nucleic acids and proteins. In an embodiment, a method of the invention may be performed by analysing a sample for a combination of protein and nucleic acid biomarkers.

Suitably, the biomarkers are proteins and the invention measures the presence or amount of each protein in a set of proteins. Suitably, the subject is a human adult.

Therefore, in a suitable embodiment, there is provided a method for determining, predicting or estimating the biological age of a subject, or for providing a measurement for use in determining, predicting or estimating the biological age of a subject, wherein the method comprises:
a) measuring, in a biological sample obtained from the subject at a first time point, the presence or amount of each protein in a set of proteins, wherein the set of proteins comprises at least 7, 8, 9, 10, 11, 12, 13, 14, 15, 16, 17, 18, 19, or 20 proteins selected from Table 1 or at least 50, 75, 100, 125, 150, 175, 200 or 204 proteins selected from Table 2.

There is also provided a method for predicting the presence or absence of at least one disease in a subject, predicting the risk of a subject of having or developing at least one disease; and/or predicting the risk of mortality of a subject, wherein the method comprises:
a) measuring, in a biological sample obtained from the subject at a first time point, the presence or amount of each protein in a set of proteins, wherein the set of proteins comprises at least 7, 8, 9, 10, 11, 12, 13, 14, 15, 16, 17, 18, 19, or 20 proteins selected from Table 1 or at least 50, 75, 100, 125, 150, 175, 200 or 204 proteins selected from Table 2.

In some embodiments, the sample is a blood-based sample such as plasma or serum and/or the subject is a human adult.

The biomarkers measured by the present invention are referred to by their names in accordance with the International Protein Nomenclature Guidelines. When a protein is measured it will be appreciated that the protein name is relevant in identifying the protein. When a nucleic acid is measured it will be appreciated that the gene name is relevant in identifying the nucleic acid. The protein names are used synonymously with the UniProt ID number provided in Tables 5 and 6. In some embodiments the proteins as recited in Tables 1, 2 and 3 are defined by the UniProt ID number as defined in Tables 5 and 6. The protein names are used synonymously with the gene name provided in Tables 5 and 6. In some embodiments the proteins as recited in Tables 1, 2 and 3 are defined by the gene name as defined in Tables 5 and 6.

A protein measured by the present invention can be a whole protein or a fragment of a protein. A fragment of a protein can contain at least 20%, 30%, 40%, 50%, 60%, 70%, 80%, 90%, 95%, 96%, 98% or 99% of the amino acid sequence of the whole protein. Suitably, a fragment comprises a contiguous length of at least 20%, 30%, 40%, 50%, 60%, 70%, 80%, 90%, 95%, 96%, 98% or 99% of the amino acid sequence of the whole protein. In some embodiments, a set of proteins comprises a combination of whole proteins and fragments of proteins.

In some embodiments a fragment of a protein measured in a method of the present invention may comprise at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 contiguous amino acids contained in an amino acid sequence of a protein recited in Table 1, 2 or 3. Suitably, a fragment of a protein is specific to the protein from which is derived, for example a fragment may comprise an epitope of the protein which is recognisable by an antibody specific to that protein.

The present invention may detect, as described herein, any form of a protein, for example splice variant (isoform), a mutant or polymorphic form, degraded and other post-translational modified forms including citrullinations, glycosylations, acetylations, phosphorylations etc.

Included within the scope of the biomarkers described herein are homologues thereof, for example structural or functional analogues and isoforms. Therefore, the present invention may detect or measure a homologue of a biomarker listed in Table 1, 2 or 3. Functional homologues are considered to be biomarkers having a different scientific name but performing the same function as one of the biomarkers listed in Table 1, 2 or 3. Structural analogues are considered to be biomarkers having a different scientific name but containing at least 70%, 80%, 90%, 95%, or 99% of the same primary, secondary, tertiary or quaternary structure as the biomarkers listed in Table 1, 2 or 3. It will be appreciated that some biomarkers will have a different name to those listed in Table 1, 2 or 3 but will perform a slightly different function or have a slightly different structure. It is intended that these similar biomarkers also fall within the scope of the biomarkers listed in Table 1, 2 or 3.

The present invention may detect, as described herein, a biomarker which may be any form of a nucleic acid, for example RNA, DNA, coding DNA (cDNA), genomic DNA (gDNA), messenger RNA (mRNA), peptide nucleic acids (PNA), Morpholino and locked nucleic acids (LNA), glycol nucleic acids (GNA), threose nucleic acids (TNA) hexitol nucleic acids (HNA). The nucleic acid may be modified by capping, cleavage, polyadenylation, intron splicing, histone processing, or methylation. Where a biomarker is a nucleic acid, suitably it may encode a protein of Table 1, 2 or 3 as provided herein, or a fragment thereof.

The set of biomarkers may be a subset of the biomarkers listed in a table provided herein. Suitably, a set of biomarkers is a subset of biomarkers provided in Table 1. More suitably the biomarkers are those found in Table 3. Suitably, the biomarkers are proteins or fragments thereof.

A method of the invention may comprise determining the presence (or absence) of each biomarker in the defined set of biomarkers, and/or determining the amount of a biomarker in the defined set of biomarkers, in a biological sample. A method of the invention further comprises the step of comparing the biomarker profile generated to a standard profile or to one or more predetermined values, one or more reference values, or to a biomarker profile generated from the same subject at a different time point, to obtain a measurement for use in determining or predicting biological age, or determining or predicting risk of disease, for example as described herein.

A measurement of the presence or amount of a biomarker in a sample obtained from a subject is suitably made at a time point. The time point may be pre-determined. A time point may refer to the time at which the sample is obtained from the subject. A time point may refer to the time at which the biomarker profile of the sample is measured. A time point may be an interval of time, for example a time point may span the time from obtaining a sample from a subject to analysing the sample according to the invention.

A method of the present invention may comprise measuring, in a further biological sample obtained from the subject at a second or further time point from step a), the presence or amount of each biomarker in the set of biomarkers; and determining the difference in the presence or amount of each biomarker in the set of biomarkers between the measurements of first, second and/or further measurements. A second or further time point may be separated from a first time point, by any suitable interval. For example, a first, second or further time points may be each separated by an interval of 1 hour, 12 hours, 24 hours, 1 month, 6 months, 1 year, 2 years, 3 years, 4 years or 5 years or more. Therefore, a method of the present invention may be performed twice or more on a subject, in order to obtain an indication of any change in the biomarker profile. A method of the invention may comprise a step of comparing a measurement with a measurement at the immediate preceding time point or a measurement of any previous time point or with a measurement taken at the first time point. A method of the present invention may comprise tracking the measurements across two or more time points for a subject. In some embodiments, the biomarkers are proteins and the invention measures the presence or amount of each protein in a set of proteins.

In certain embodiments the method of the invention further comprises contacting each of the biomarkers in the set of biomarkers disclosed herein with a plurality of antibodies wherein each antibody specifically binds to and recognises one of the biomarkers of the set of biomarkers. In some embodiments, the antibody is suitable for a proximity extension assay. In some embodiments the method further comprises measuring the amount of binding between the antibody and the biomarker to determine the presence or amount of the biomarkers in a biological sample. In some embodiments, the biomarkers are proteins and the invention measures the presence or amount of each protein in a set of proteins.

The method can further comprise comparing the presence or amount of the biomarkers in the biological sample with predetermined threshold values, wherein levels of expression of at least one of the plurality of biomarkers above or below the predetermined threshold values is indicating of the biological age of a subject or the presence or absence of at least one disease in a subject, or the risk of a subject of having or developing at least one disease; and/or the risk of mortality of a subject.

The present invention can measure the amount of biomarkers. As used herein, amount may refer to the absolute amount of a biomarker, for example the concentration of a biomarker in a biological sample. The amount of a biomarker may also refer to a relative amount of the biomarker, for example a relative difference versus a reference measurement. The reference measurement may be the same biomarker within a larger population of subjects, the amount of another biomarker, the same biomarker at a different time point, the amount of another biomarker, or any other value such as an amount of DNA methylation levels, single nucleotide polymorphisms (SNPs) levels, telomere length, or other cellular senescence biomarkers. The amount of a biomarker may be a single measurement or may be a value associated with a change over time in the amount of said biomarker. In some embodiments, amount refers to the concentration of each biomarker in a set of biomarkers. In some embodiments, amount refers to the abundance of each biomarker in a set of biomarkers relative to other biomarkers in the set of biomarkers. In some embodiments, the invention measures the presence or amount of each protein in a set of proteins.

A method of the invention may be for determining, predicting or estimating the biological age of a subject, or for providing a measurement for use in determining, predicting or estimating the biological age of a subject. Such a measurement may be useful in predicting the risk of disease, suitably age-related disease, in the subject. A method of the present invention may also be used for predicting the presence or absence of at least one disease in a subject, predicting the risk of a subject of having or developing at least one disease; and/or predicting the risk of mortality of a subject.

As used herein, age-related disease refers to any disease that is associated with increased frequency and/or severity in subjects with a greater chronological age or biological age. In some embodiments, an age-related disease is one that occurs more frequently in subjects with increased chronological age. This can be in subjects that are 20 years or older, 30 years or older, 40 years or older, 50 years or older, 60 years or older, 70 years or older, 80 years or older, 90 years or older or 100 years or older, compared to younger subjects. In some embodiments the younger subjects are at least 5, 10, 15, 20, 30, 40, 50, 60, 70 or 80 years younger than the subject with a greater chronological age. The disease may be a chronic disease or an acute disease. Herein, disease, suitably an age-related disease, may be selected from chronic liver disease, type II diabetes, Parkinson's disease, rheumatoid arthritis, osteoarthritis, macular degeneration, ischemic heart disease, stroke, osteoporosis, ischemic stroke, emphysema, chronic obstructive pulmonary disease (COPD), chronic kidney diseases, all-cause dementia, Alzheimer's disease, oesophageal cancer, prostate cancer, lung cancer, non-Hodgkin lymphoma or combinations thereof. The symptoms and diagnostic methods for these diseases are known in the art.

Examples of suitable probes include antibodies, antibody fragments, oligonucleotides, proteins, biotin-binding proteins, enzymes, fluorophores, aptamers, primers or combinations thereof. Specific combinations of probes can include antibodies and antibody fragments. Specific examples of oligonucleotides include DNA and RNA probes. In some embodiments a combination of DNA and RNA probes are used. In preferred embodiments, the biomarkers are proteins and the probes are antibodies. In some embodiments the antibodies are suitable for ELISA or proximity extension assay.

Herein, a set of probes for detecting a set of biomarkers, as described in the methods of the invention, may include a probe specific for detection of a single biomarker in the panel of biomarkers (e.g. the selected proteins of Table 1, 2 or 3), such that each biomarker in the set can be individually detected. For example, where there is a panel of 10 biomarkers to be detected in a sample, a set of probes will suitably comprise 10 probes, one probe specific for each biomarker. The probes must differ in terms of specificity for the biomarkers, but may each be the same or different types of probe, for example antibody, nucleic acid etc. A set of probes may include one type of probe (e.g. an antibody) for detection of each biomarker in the set of biomarkers. A set of probes may include more than one type of probe (three, four, five, six, or more types of probe) for detection of each biomarker in the set of biomarkers. Suitably, each probe is specific for one biomarker. It will be appreciates that there will be multiple copies of each probe, and reference herein to “each” probe or “a” probe of the set refers to the specificity of the probe. Typically, the number of probes in a set will correlate to the number of biomarkers in the set.

In a suitable embodiment, a method of the invention may be an antibody based assay.

Therefore, in a suitable embodiment, there is provided an ELISA assay or proximity extension assay for determining, predicting or estimating the biological age of a subject, or for providing a measurement for use in determining, predicting or estimating the biological age of a subject, wherein the method comprises:
a) measuring, in a biological sample obtained from the subject at a first time point, the presence or amount of each protein in a set of proteins, wherein the set of proteins comprises at least 7, 8, 9, 10, 11, 12, 13, 14, 15, 16, 17, 18, 19, or 20 proteins selected from Table 1 or at least 50, 75, 100, 125, 150, 175, 200 or 204 proteins selected from Table 2.

There is also provided an ELISA assay or proximity extension assay for predicting the presence or absence of at least one disease in a subject, predicting the risk of a subject of having or developing at least one disease; and/or predicting the risk of mortality of a subject, wherein the method comprises:
a) measuring, in a biological sample obtained from the subject at a first time point, the presence or amount of each protein in a set of proteins, wherein the set of proteins comprises at least 7, 8, 9, 10, 11, 12, 13, 14, 15, 16, 17, 18, 19, or 20 proteins selected from Table 1 or at least 50, 75, 100, 125, 150, 175, 200 or 204 proteins selected from Table 2.

In some embodiments, the biological sample is a blood-based sample such as serum or plasma and/or the subject is a human adult.

An antibody may be naturally occurring and non-naturally occurring antibodies, including a wholly synthetic antibody. An antibody may be monoclonal, polyclonal or recombinant, chimeric and humanized antibodies. An antibody may be human or non-human. A nonhuman antibody can be humanized by recombinant methods to reduce its immunogenicity in man (i.e. to produce a humanized antibody). An antibody may be. An antibody may include a single chain antibody. An antibody includes any immunoglobulin (e.g., IgG, IgM, IgA, IgE, IgD, etc.) obtained from any source (e.g., humans, rodents, nonhuman primates, caprines, bovines, equines, ovines, etc.). Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain antibody.

In an antibody based assay of the invention, an antibody may be measured directly wherein the antibody is conjugated with an enzyme or fluorescent dye for direct detection. The antibody may be measured indirectly in which an unlabelled primary antibody is detected using an enzyme- or fluorophore-conjugated secondary antibody. A probe may also be a fragment of an antibody disclosed herein. Examples of suitable antibody fragments include F(ab′)2, Fab, Fab′ and Fv. These can be generated from the variable region of IgG and IgM.

These antigen-binding fragments vary in size (MW), valency and Fc content. Fc fragments are generated entirely from the heavy chain constant region of an immunoglobulin. These and several additional unique fragment structures can be generated from pentameric IgM, including an “IgG”-type fragment, an inverted “IgG”-type fragment, and a pentameric Fc fragment.

A probe/detection agent may be labelled with a detectable moiety. Suitable detectable moieties may be selected from the group consisting of luminescent agents, chemiluminescent agents, radioisotopes, colorimetric agents; and enzyme-substrate agents. In preferred embodiments the probes are antibodies coupled to unique DNA sequence tags. In preferred embodiments the probe/detection agent is for use in a proximity extension assay which is known in the art.

A nucleic acid probe/detection agent may include triple-, double- and single-stranded DNA, as well as triple-, double- and single-stranded RNA. A nucleic acid probe may be a modified form, for example by methylation and/or by capping, or an unmodified form of the polynucleotide. A nucleic acid probe may include polydeoxyribonucleotides (containing 2-deoxy-D-ribose), polyribonucleotides (containing D-ribose), and any other type of polynucleotide which is an N- or C-glycoside of a purine or pyrimidine base. A nucleic acid probe may be any suitable length, for example about 20, 50, 100, 200, 500, 1000, or 1500 bases long.

Oligonucleotide probes for protein detection can involve nucleic acid-based fluorescence probe for protein detection and are known in the art. An oligonucleotide probe may be DNA, RNA, and include antisense oligonucleotides (ASO), RNA interference (RNAi), and aptamer RNAs. Some oligonucleotides can detect proteins by scission of an aptamer into two probes, which are then attached with a chemically reactive fluorogenic compound. The protein-dependent association of the two probes accelerates a chemical reaction and indicates the presence of the target protein, which is detected using a fluorescence readout.

Biotin-binding protein probes use fluorescent conjugates of streptavidin to detect biotinylated biomolecules such as primary and secondary antibodies, ligands and toxins, or DNA probes for in situ hybridization or bead-based detection. Enzyme conjugates of streptavidin, such as HRP and AP, are commonly used in western blotting, ELISA, and in situ hybridization imaging applications. Streptavidin-conjugated magnetic beads and resins can be used to isolate proteins, cells, and DNA, or they can be used in immunoassays or bio-panning.

Enzymatic probes, such as horseradish peroxidase (HRP) and alkaline phosphatase (AP), can be used to detect target proteins through chromogenic, chemiluminescent or fluorescent outputs. The variability of these readouts demonstrates the versatility that enzymatic probes have in biological research methods, including immunohistochemistry (IHC), immunoblotting and enzyme-linked immunosorbent assays (ELISAs). Such enzymatic probes and typically conjugated to an antibody or other suitable detecting agent that specifically binds to and recognises the biomarkers of interest.

The use of fluorescent molecules in biological research is the standard in many applications, and their use is continually increasing due to their versatility, sensitivity and quantitative capabilities. Among their myriad of uses, fluorescent probes are employed to detect protein location and activation, identify protein complex formation and conformational changes and monitor biological processes. Examples of fluorescent probes include fluorescent proteins not normally expressed in the subject, including but not limited to green fluorescent protein (GFP), red fluorescent protein (RFP), yellow fluorescent protein (RFP), mCherry, blue fluorescent protein (BFP), cyan fluorescent protein (CFP).

When the biomarker is a protein, a variety of different methods of assaying protein levels are known to one of ordinary skill in the art, and any convenient method may be used. Representative exemplary methods include but are not limited to antibody-based methods (e.g., immunofluorescence assay, radioimmunoassay, immunoprecipitation, Western blotting, proteomic arrays, xMAP microsphere technology (e.g., Luminex technology), immunohistochemistry, flow cytometry, and the like) as well as non-antibody-based methods (e.g., mass spectrometry or tandem mass spectrometry). Examples of mass spectrometers are time-of-flight, magnetic sector, quadrupole filter, ion trap, ion cyclotron resonance, Orbitrap, hybrids or combinations of the foregoing, and the like. In another embodiment, the method comprises the use of MALDI-TOF tandem mass spectrometry (MALDI-TOF MS/MS).

Two representative and convenient techniques for assaying protein levels in a sample include aptamer-based assays and antibody-based methods such as the enzyme-linked immunosorbent assay (ELISA). Aptamer-based assays use aptamers comprising single-stranded oligonucleotides that bind specifically to biomarker proteins of interest. Either high affinity RNA aptamers or DNA aptamers with specificity for a protein of interest may be used. Functional groups that mimic amino acid side-chains may be added to aptamers to confer protein-like properties to improve binding affinity to a protein of interest. Aptamers that bind specifically and with high affinity to a biomarker protein of interest can be selected from large libraries of aptamers having randomized sequences using Systematic Evolution of Ligands by Exponential enrichment (SELEX). The aptamers may be designed with unique nucleotide sequences recognizable by specific hybridization probes for capture on a hybridization array for multiplexed detection of biomarkers.

Where mass spectrometry is used in a method of the invention, the method may comprise a step of protein digestion e.g. trypsin digestion. The method may include fractionation, for example by capture on a chromatographic resin or cation exchange resin. Alternatively, the method could be preceded by fractionating the sample on an anion exchange resin before application to the cation exchange resin.

The present invention can use a multiplex assay for detecting multiple biomarkers in a single assay, e.g. in a single reaction using a single sample such that two or more biomarkers may be detected simultaneously. An example of a suitable multiplex assay is a proximity extension assay. Alternatively, the present invention can use separate assays or reactions for each biomarker of a sample, such that the detection of each biomarker is performed in a separate reaction. The separate reactions may be performed simultaneously, for example in an array. An example of an embodiment where a single biomarker is detected in a reaction is an ELISA. For any sample, a combinations of multiplex and separate assays can be used.

Where the invention comprises two or more separate reactions to detect the presence or absence or amount of a set of biomarkers, the reactions may be performed spatially separately, using distinct reaction locations. The reactions may alternatively or additionally be performed temporally separately, for example wherein two or more biomarker assays are performed at different time points, e.g one after the other. In some embodiments the reactions are performed spatially separate and temporally separate, for example in sequential batches.

In some preferred embodiments the detection method for a protein is a proximity extension assay. A proximity extension assay (PEA) is a method for detecting and quantifying the amount of many specific proteins present in a biological sample such a serum or plasma. The method is used in the research field of proteomics, specifically affinity proteomics, wherein one searches for differences in the abundance of many specific proteins in blood for use as a biomarker. PEA is performed without a solid phase in a homogeneous one tube reaction solution where in sets of antibodies coupled to unique DNA sequence tags, so called proximity probes, work in pairs specific for each target protein. PEA is often performed using antibodies and is a type of immunoassay. Target binding by the proximity probes increases their local relative effective concentration of the DNA-tags enabling hybridization of weak complementarity to each other which then enables a DNA polymerase mediated extension forming a united DNA sequence specific for each target protein detected. The use of 3′exonuclease proficient polymerases lowers background noise and hyper thermostable polymerases mediate a simple assay with a natural hot-start reaction. This created pool of extension products of DNA sequence forms amplicons amplified by PCR where each amplicon sequence corresponds to a target proteins identity and the amount reflects its quantity. Subsequently, these amplicons are detected and quantified by either real-time PCR or next generation DNA sequencing by DNA-tag counting. PEA enables the detection of many proteins simultaneously (so called multiplexing) due to the readout requiring the combination of two correctly bound antibodies per protein to generate a detectable DNA sequence from the extension reaction. Only cognate pairs of sequence are detected as true signal. The DNA amplification power also enable minute sample volumes even below one microliter.

Suitably when the detection method is PEA, the step of (a) measuring, in a biological sample obtained from the subject at a first time point, the presence or amount of each biomarker in a set of biomarkers can comprise the steps of:
i) contact a biological sample from the subject with blocking antibodies to prevent nonspecific binding of proximity probes
ii) incubating the mixture of step (i) with proximity extension assay probe pairs specific to each biomarker in the set of biomarkers
iii) performing a DNA polymerase driven DNA extension assay to extend the dimerised oligomer tags on the proximity extension assay probe pairs when they are in proximity to produce DNA products specific to each biomarker in the set of biomarkers
iv) detecting the DNA products specific to each biomarker in the set of biomarkers by polymerase chain reaction;
wherein the biomarkers are proteins or fragments thereof.

When the biomarker is a nucleic acid, a variety of different methods of assaying nucleic acid levels are known to one of ordinary skill in the art, and any convenient method may be used.

Polymerase chain reaction (PCR) can be used when the biomarker is a nucleic acid. For example, the PCR may be quantitative type PCR, such as quantitative, real-time PCR (both singleplex and multiplex). Therefore, a method of the invention may comprise the steps of contacting nucleic acid of the biological sample with one or more primers that specifically bind one or more biomarker described herein, to form a primer:biomarker complex; maintaining the nucleic acid under conditions to allow the primers to hybridise to the nucleic acid of the biological sample; and amplifying the primer:biomarker complexes. The conditions may be stringent hybridisation conditions. The amplified complexes can then be detected/quantified to determine a level of expression of the one or more biomarkers.

Therefore, in a suitable embodiment, there is provided a method of polymerase chain reaction for determining, predicting or estimating the biological age of a subject, or for providing a measurement for use in determining, predicting or estimating the biological age of a subject, wherein the method comprises:
a) measuring, in a biological sample obtained from the subject at a first time point, the presence or amount of each biomarker in a set of biomarkers, wherein the set of biomarkers comprises at least 7, 8, 9, 10, 11, 12, 13, 14, 15, 16, 17, 18, 19, or 20 biomarkers selected from Table 1 or at least 50, 75, 100, 125, 150, 175, 200 or 204 biomarkers selected from Table 2.

There is also provided a method of polymerase chain reaction for predicting the presence or absence of at least one disease in a subject, predicting the risk of a subject of having or developing at least one disease; and/or predicting the risk of mortality of a subject, wherein the method comprises:
a) measuring, in a biological sample obtained from the subject at a first time point, the presence or amount of each biomarker in a set of biomarkers, wherein the set of biomarkers comprises at least 7, 8, 9, 10, 11, 12, 13, 14, 15, 16, 17, 18, 19, or 20 biomarkers selected from Table 1 or at least 50, 75, 100, 125, 150, 175, 200 or 204 biomarkers selected from Table 2.

Suitably when the detection method is PCR, the step of (a) measuring, in a biological sample obtained from the subject at a first time point, the presence or amount of each biomarker in a set of biomarkers can comprise the steps of:
i) contacting a biological sample from the subject with primers specific to each biomarker in the set of biomarkers
ii) performing repeated steps of DNA amplification to produce DNA extension products specific to each biomarker in the set of biomarkers
iii) detecting the DNA extension products specific to each biomarker in the set of biomarkers to quantify the amounts of each biomarkers in the set of biomarkers in biological sample;
   wherein the biomarkers are nucleic acids or fragments thereof.

In some embodiments the subject is a human adult and/or the biomarker is a gene product of one of the biomarkers disclosed in Tables 1, 2, or 3 and/or the biological sample is a blood-based sample such and plasma or serum.

In some embodiments of the invention the method comprises comparing the amount of the biomarkers in a set of biomarkers against a reference measurement obtained from a subject of a known age or disease status. As used herein, reference subject or reference measurement refers to a measured presence or amount of a biomarker that has been correlated with a known disease status or severity, or known chronological age or biological age in a subject or in a group of subjects. The reference measurement may be a single value or a set of values, for example a value for each biomarker. The reference measurement may be a range. Suitably, a reference measurement is from UK Biobank samples, FinnGen samples, China Kadoorie Biobank samples or combinations thereof.

The method of the invention may include a step of comparing measurement of presence or amount for each biomarker with reference values for each biomarker. The method may include assessing whether the presence or level of one or more biomarkers of the set in a sample from a patient is the same as, more or less than, different from levels of the same biomarkers in a control or reference sample or a reference value. In some embodiments, the biomarkers are proteins and the invention measures the presence or amount of each protein in a set of proteins.

In some embodiments the subject is assigned a numerical biological age determined by the presence or amount of the biomarkers in the set of biomarkers. This can be determined by a statistical or machine learning model that uses information on the presence or amount of the biomarkers to predict chronological age or to predict a previously calculated physiological age phenotype. In some embodiments, the biomarkers are proteins and the invention measures the presence or amount of each protein in a set of proteins. In some embodiments, the subject is assigned a numerical biological age based on the presence or amount of the biomarkers in the set of biomarkers.

In some embodiments, the relationship between the presence or amount of the biomarkers in the set of biomarkers is the correlation between the presence or amount of each of the biomarkers in the set of biomarkers.

The prediction made according to some method of the invention allows for assessing whether the probability is high and, thus, it is expected that a subject has a disease or a particular severity of a disease, or whether the probability is low and, thus, it is expected that a subject does not have a disease or a particular severity of a disease. This is determined by calculating the relationship between the presence or amount of the biomarkers in the set of biomarkers in a reference measurement and the presence or amount of the biomarkers in the set of biomarkers in subjects in need of prediction. The prediction can be of the presence or absence of at least one disease in the subject, the risk of the subject of having or developing at least one disease; and/or the risk of mortality of the subject. In some embodiments, the invention measures the presence or amount of each protein in a set of proteins.

A method of the present invention may comprise obtaining information about the subject, including for example chronological age, sex, race, nationality, residence, health status, functional measurements, blood biochemistry values etc. One or more of these data may be used in estimating the biological age or comparing with the biological age to provide a determination or prediction relating to disease as described herein.

A device of the present invention comprises the probes as disclosed herein. In some embodiments the device is for performing a proximity extension assay. In these embodiments, the device comprises a set of antibodies that specifically bind to and recognise each of the proteins in a set of proteins wherein the set of proteins comprises at least 7, 8, 9, 10, 11, 12, 13, 14, 15, 16, 17, 18, 19, or 20 proteins selected from Table 1 or at least 50, 75, 100, 125, 150, 175, 200 or 204 proteins selected from Table 2. In certain embodiments the device comprises a set of antibodies that comprises at least two antibodies that bind to each protein in the set of proteins and are conjugated to complementary DNA tags such that proximity of the antibodies occurs when both antibodies bind to the same proteins and the complementary DNA tags can hybridise and allows DNA polymerase mediated extension of the hybridised DNA tag. The device can further comprise reagents for detecting the DNA polymerase mediated extension product of the hybridised DNA tag.

In some embodiments the device is for performing an enzyme-linked immunosorbent assay (ELISA). In these embodiments, the device comprises a set of antibodies wherein each antibody specifically binds to and recognises a proteins in a set of proteins wherein the set of proteins comprises at least 7, 8, 9, 10, 11, 12, 13, 14, 15, 16, 17, 18, 19, or 20 proteins selected from the biomarkers of Table 1 or at least 50, 75, 100, 125, 150, 175, 200 or 204 proteins selected from the biomarkers of Table 2. Certain embodiments further comprise at least one of suitable buffers, wash solution, microwell plate, instructions, reference chart or combinations thereof. In an ELISA assay, the antigen is immobilized to a solid surface. The device or method of the present invention may be for performing an ELISA. The ELISA may be direct, indirect, sandwich, or competitive. Such methods and devices are known in the art.

In some embodiments the device is for performing a PCR analysis. In these embodiments, the device comprises a set of primers wherein each primer is specific for one of the biomarkers in a set of biomarkers wherein the set of biomarkers comprises at least 7, 8, 9, 10, 11, 12, 13, 14, 15, 16, 17, 18, 19, or 20 biomarkers selected from the biomarkers of Table 1 or at least 50, 75, 100, 125, 150, 175, 200 or 204 biomarkers selected from the biomarkers of Table 2. The device can further comprise reagents for performing a PCR reaction including DNA polymerase, a thermocycler, dNTPs, buffers, and a detection reagent. The detection reagent may bind at all double-stranded DNA or may be specific to the amplicons of each biomarker in the set of biomarkers.

In some embodiments the devices as disclosed herein further comprise at least one of the following nitrocellulose membranes, fractionation columns, protein binding columns, protein affinity columns, protein purification columns, magnetic beads, labelled beads, tagged beads, 96-well plates, 384-well plates, microtiter plates, biochips (biochips generally comprise solid substrates and have a generally planar surface, to which a capture reagent), buffers. In some embodiments the device of the present invention further comprises a solid substrate to which the probes can be immobilised on. The probe may be permanently immobilized or reversibly immobilized. The solid substrate can be the well of a plate, a bead, a membrane, or combinations thereof.

In some embodiments the device of the present invention further comprises a solid substrate and a plurality of binding agents immobilized on the substrate, wherein each of the binding agents is immobilized at a different, indexable, location on the substrate and the binding agents specifically bind to a plurality of biomarkers.

In some embodiments of the invention them is provided a kit comprising the probes disclosed herein and suitable sampling equipment. Suitably, the sampling equipment is for blood sampling. Sampling equipment may include at least one of a lancet, plaster, pre-injection swab, name label, gauze swab, a protective packing wallet, blood collection tube, a pre-paid return envelope, or a combination thereof. Where a kit is for home use, it may comprise a suitable device for detection of the presence or absence or amount of a set of biomarkers as described herein. Such a device may be disposable. A kit of the invention may also include instructions for use. A kit of the invention may also include a reference chart for comparison with the assay results.

In some embodiments, there is provided a computer-implemented method of determining, predicting or estimating the biological age of a subject comprising the steps of:
a) obtaining data of the measured levels of: i) at least 7 biomarkers in Table 1 in claim 1; or ii) at least 50 biomarkers in Table 2 of claim 2;
b) Inputting the measured levels in step a) to a predictive model which relates the measured levels with biological age or chronological age; and
c) Outputting a determined, predicted or estimated biological age.

The method may be performed using measured levels taken at different time points. The method may additionally compute the relationship between chronological age and the biological age of the subject to determine or estimate a value of an age gap or accelerated/decelerated aging. By relate is meant the model finds the relationship between the input and the output.

By computer program is meant machine readable program instructions. These may be provided on a transitory medium such as a transmission medium or on a non-transitory medium such as a storage medium. Such machine readable instructions (computer program code) may be implemented in a high level procedural or object oriented programming language. However, the program(s) may be implemented in assembly or machine language, if desired. In any case, the language may be a compiled or interpreted language, and combined with hardware implementations. Program instructions may be executed on a single processor or on two or more processors in a distributed manner.

In some embodiments there is provided a data processing apparatus comprising means of carrying out the computer-implemented method. The processing circuitry of the apparatus may be communicatively coupled to a memory. The memory may store the machine learning model. The processing circuitry may comprise general purpose processor circuitry configured by program code to perform specified processing functions. Alternatively, the processing circuitry may comprise special purpose processing circuitry. Thus, the configuration of the circuitry to perform its specified function may be limited exclusively to hardware, limited exclusively to software, or a combination of hardware modification and software execution.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

The protein expression data generated from the Olink Explore 3072 panel is used in this invention. Data generated from this panel are provided in Olink's Normalized Protein eXpression (NPX) format. According to Olink, this means that NPX values can be compared only for the same protein across the samples analyzed in a single occasion and cannot be compared across projects run at separate occasions without the use of reference bridging samples. Despite this stated limitation by Olink, the inventors have developed and employed a statistical and analytical technique to normalize the protein data across biobanks with no bridging samples. With this approach, they have been able to develop a model in one population and validate it in a completely new population without bridging samples.

The invention is described herein by way of non-limiting examples and with reference to the drawings.

### EXAMPLES

In the following, the invention will be explained in more detail by means of non-limiting examples of specific embodiments. In the example experiments, standard reagents and buffers free from contamination are used.

### Example 1—Methods

### Study Populations

The UK Biobank (UKB) is a prospective cohort study with extensive genetic, metabolomic and proteomic and phenotype data available for 502,505 individuals resident in the United Kingdom who were recruited from 2006-2010 (Sudlow et al. 2015). The inventors restricted the UKB sample to those participants with Olink Explore 3072 data available at baseline who were randomly sampled from the main UKB population (n=45,441).

The China Kadoorie Biobank (CKB) is a prospective cohort study of 512,724 adults aged 30-79 years who were recruited from ten geographically diverse (five rural and five urban) areas across China during 2004-2008. Details on the CKB study design and methods have been previously reported (Chen et al. 2011). The inventors restricted the CKB sample to those participants with Olink Explore 3072 data available at baseline in a nested case-cohort study of ischemic heart disease and who were genetically unrelated to each other (n=3,977).

The FinnGen study is a public-private partnership research project that has collected and analyzed genome and health data from 500,000 Finnish biobank donors to understand the genetic basis of diseases (Kurki et al. 2023). FinnGen includes 9 Finnish biobanks, research institutes, universities and university hospitals, 13 international pharmaceutical industry partners and the Finnish Biobank Cooperative (FINBB). The project utilizes data from the nationwide longitudinal health register collected since 1969 from every resident in Finland. In FinnGen, the inventors restricted the analyses to those participants with Olink Explore 3072 data available and passing proteomics data quality control (QC) (n=1,990).

### Proteomic Profiling

Proteomic profiling in the UKB, CKB, and FinnGen was carried out for protein analytes measured via the Olink Explore 3072 platform that links four Olink panels (Cardiometabolic, Inflammation, Neurology, and Oncology). The random subsample of UKB proteomics participants (n=45,441) were selected by removing those in batches 0 and 7. Randomized participants selected for proteomic profiling in the UKB have been shown previously to be highly representative of the wider UKB population (Sun et al. 2023). UKB Olink data are provided Normalized Protein eXpression (NPX) values on a log 2 scale, with details on sample selection, processing, and quality control documented online.

In the CKB, stored baseline plasma samples from participants were retrieved, thawed, and sub-aliquoted into multiple aliquots, with one (100 μL) aliquot used to make two sets of 96-well plates (40 μL/well). Both sets of plates were shipped on dry ice, one to the Olink Bioscience Laboratory at Uppsala, Sweden (batch 1, 1463 unique proteins) and the other shipped to the Olink laboratory in Boston, USA (batch 2, 1460 unique proteins), for proteomic analysis using a multiplex proximity extension assay, with each batch covering all 3,977 samples. Samples were plated in the order they were retrieved from long-term storage at the Wolfson laboratory in Oxford, UK and normalized using both an internal control (extension control) and an inter-plate control and then transformed using a pre-determined correction factor. The limit of detection (LOD) was determined using negative control samples (buffer without antigen). A sample was flagged as having a QC warning if the incubation control deviated more than a pre-determined value (t 0.3) from the median value of all samples on the plate (but values below LOD were included in the analyses). The pre-processed data were provided in the arbitrary NPX unit on a log 2 scale.

In the FinnGen study, blood samples were collected from healthy individuals and EDTA-plasma aliquots (230 μL) were processed and stored at −80° C. within 4 hours. Plasma aliquots were subsequently thawed and plated in 96-well plates (120 μL/well) as per Olink's instructions. Samples were shipped on dry ice to the Olink Bioscience Laboratory (Uppsala, Sweden) for proteomic analysis using the 3072 multiplex proximity extension assay. Samples were sent in three batches and to minimize any batch effects, bridging samples were added according to Olink's recommendations. In addition, plates were normalized using both an internal control (extension control) and an inter-plate control and then transformed using a pre-determined correction factor. The limit of detection (LOD) was determined using negative control samples (buffer without antigen). A sample was flagged as having a QC warning if the incubation control deviated more than a pre-determined value (±0.3) from the median value of all samples on the plate (but values below LOD were included in the analyses). The pre-processed data were provided in the arbitrary NPX unit on a log 2 scale.

The inventors excluded from analysis any proteins not available in all three cohorts, as well as an additional three proteins that were missing in over 10% of the UKB sample (CTSS, PCOLCE, NPM1), leaving a total of 2,897 proteins for analysis. After missing data imputation (see below), proteomic data was re-normalized separately within each cohort by first rescaling values to be between 0-1 using MinMaxScaler( ) from scikit-learn and then centering on the median. This approach allowed for NPX data from one cohort or population to be related to another, and allowed for predictions to be made in new NPX data using models trained from NPX data in other cohorts or populations.

### Outcomes

UKB aging biomarkers were measured using baseline non-fasting blood serum samples as previously described (Elliott and Peakman 2008). Biomarkers were previously adjusted for technical variation by the UKB, with sample processing and quality control procedures described on the UK Biobank website. Field IDs for all biomarkers and measures of physical and cognitive decline are shown in Table 22. Poor self-rated health, slow walking pace, self-rated facial aging, feeling tired/lethargic every day, and frequent insomnia were all binary dummy variables coded as all other responses versus responses for “Poor” (overall health rating; Field ID 2178), “Slow pace” (usual walking pace; Field ID 924), “Older than you are” (facial aging; Field ID 1757), “Nearly every day” (frequency of tiredness/lethargy in last 2 weeks; Field ID 2080), and “Usually” (sleeplessness/insomnia; Field ID 1200), respectively. Sleeping 10+ hours/day was coded as a binary variable using the continuous measure of self-reported sleep duration (Field ID 160). Systolic and diastolic blood pressure were averaged across both automated readings. Standardized lung function (FEV1) was calculated by dividing the FEV1 best measure (field ID 20150) by standing height squared (field ID 50). Hand grip strength variables (field ID 46,47) were divided by weight (Field ID 21002) to normalize according to body mass. Frailty index was calculated using the algorithm previously developed for UK Biobank data by Williams et al. (2019). Components of the frailty index are shown in Table 23. Leukocyte telomere length was measured as the ratio of telomere repeat copy number (T) relative to that of a single copy gene (S, HBB, which encodes human hemoglobin subunit B) (Codd et al. 2022). This T/S ratio was adjusted for technical variation and then both log-transformed and Z-standardized using the distribution of all individuals with a telomere length measurement.

Detailed information about the linkage procedure with national registries for mortality and cause of death information in the UKB is available online. Mortality data were accessed from the UKB data portal on May 23, 2023, with a censoring date of Nov. 30, 2022 for all participants (12-16 years of follow-up).

Data used to define prevalent and incident chronic diseases in the UKB are outlined in Table 24. In the UKB, incident cancer diagnoses were ascertained using ICD diagnosis codes and corresponding dates of diagnosis from linked cancer and mortality register data. Incident diagnoses for all other diseases were ascertained using ICD diagnosis codes and corresponding dates of diagnosis taken from linked hospital inpatient, primary care, and mortality register data. Primary care read codes were converted to corresponding ICD diagnosis codes using the lookup table provided by the UKB. Linked hospital inpatient, primary care, and cancer register data were accessed from the UKB data portal on May 23, 2023, with a censoring date of Oct. 31, 2022; Jul. 31, 2021; or Feb. 28, 2018 for participants recruited in England, Scotland, or Wales, respectively (8-16 years of follow-up).

In the CKB, information about incident disease and cause-specific mortality was obtained by electronic linkage, via the unique national identification number, to established local mortality (cause-specific) and morbidity (for stroke, IHD, cancer and diabetes) registries and to the health insurance system that records any hospitalization episodes and procedures (Chen et al. 2005, Chen et al. 2011). All disease diagnoses were coded using the Tenth International Classification of Diseases (ICD-10), blinded to any baseline information and participants were followed up to death, loss-to-follow-up or the 1 Jan. 2019. ICD-10 codes used to define diseases studied in the CKB are shown in Table 25.

### Missing Data Imputation

Missing values for all non-proteomics UKB data were imputed using the R package missRanger (Mayer et al. 2019), which combines random forest imputation with predictive mean matching. The inventors imputed a single dataset using a maximum of 10 iterations and 200 trees. All other random forest hyperparameters were left at their default. The imputation dataset included all baseline variables available in the UKB as predictors for imputation, excluding variables with any nested response patterns. Responses of “do not know” were set to NA and imputed. Responses of “prefer not to answer” were not imputed and set to NA in the final analysis dataset. Age and incident health outcomes were not imputed in the UKB. CKB data had no missing values to impute.

Protein expression values were imputed in the UKB and FinnGen cohort using the miceforest package in Python. All proteins except those missing in >30% of participants were used as predictors for imputation of each protein. The inventors imputed a single dataset using a maximum of 5 iterations. All other parameters were left at their default.

### Calculation of Chronological Age Measures

In the UKB, the inventors derived a more precise estimate of chronological age, since age at recruitment (field ID 21022) is only provided as a whole integer value. This was done by taking month of birth (field ID 52) and year of birth (field ID 34) and creating an approximate date of birth for each participant as the first day of their birth month and year. Age at recruitment as a decimal value was then calculated as the number of days between each participant's recruitment date (field ID 53) and approximate birth date divided by 365.25. Age at the first imaging follow-up (2014+) and the repeat imaging follow-up (2019+) were then calculated by taking the number of days between the date of each participant's follow-up visit and their initial recruitment date divided by 365.25 and adding this to age at recruitment as a decimal value. Recruitment age in the CKB is already provided as a decimal value.

### Model Benchmarking

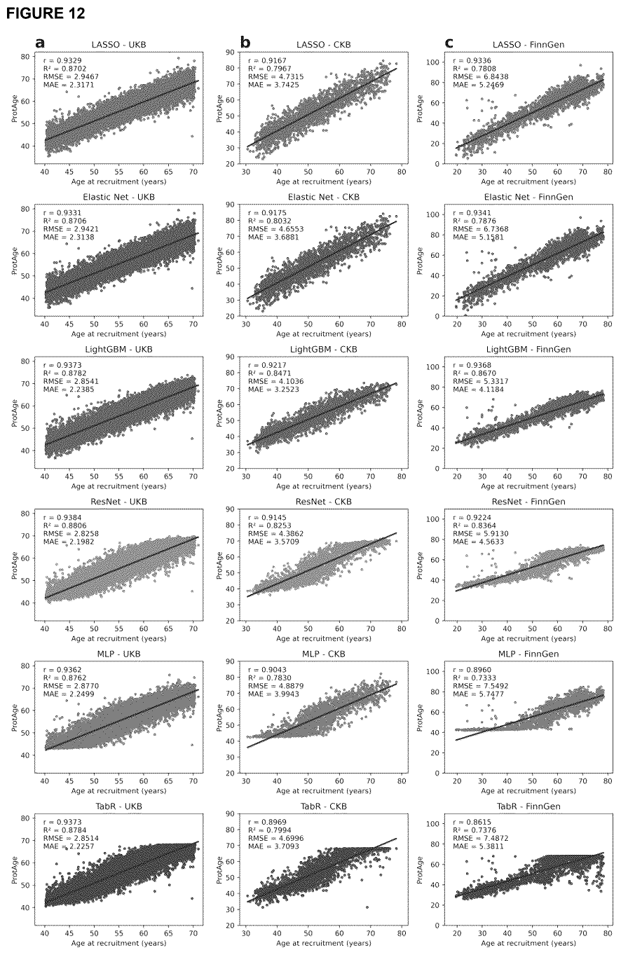

The inventors compared the performance of 6 different machine learning models (LASSO, elastic net, LightGBM, and three neural network architectures: multilayer perceptron [MLP], ResNet, and TabR) for using plasma proteomics data to predict age. For each model, the inventors trained a regression model using all 2,897 Olink protein expression variables as input to predict chronological age. All models were trained using 5-fold cross validation in the UK Biobank training data (n=31,808) and were tested against the UKB holdout test set (n=13,633), as well as independent validation sets from the CKB and FinnGen cohorts. The inventors found that LightGBM provided the 2nd best model accuracy among the UKB test set, but showed significantly better performance in the independent validation sets (FIG. 12).

LASSO and elastic net models were calculated using the scikit-learn package in python. For the LASSO model, the inventors tuned the alpha parameter using the LassoCV function and an alpha parameter space of [1e-15, 1e-10, 1e-8, 1e-5,1e-4, 1e-3,1e-2, 1, 5, 10, 50, 100]. Elastic net models were tuned for both alpha (using the same parameter space) and L1 ratio drawn from the following possible values: [0.1, 0.5, 0.7, 0.9, 0.95, 0.99, 1].

The LightGBM model hyperparameters were tuned via 5-fold cross-validation using the Optuna module in Python (Akiba et al. 2019), with parameters tested across 200 trials and optimized to maximize the average R² of the models across all folds.

The neural network (NN) architectures tested in this analysis were selected from a list of architectures that performed well on a variety of tabular datasets [1, 2]. The architectures considered were: (i) a multilayer perceptron (MLP); (ii) a residual feedforward network (ResNet); and (iii) a retrieval-augmented neural network for tabular data (TabR). Similar to the other models, each NN model utilized the concentration of 2,897 proteins as input and trained via a regression model to predict biological age. All NN model hyperparameters were tuned via 5-fold cross-validation using Optuna across 100 trials and optimized to maximize the average R² of the models across all folds.

The MLP architecture is the simplest NN architecture with multiple layers of neurons stacked on each other, and the information flows in a feedforward manner from the input features to the predicted output. Dropout (randomly dropping out nodes during training) is introduced between each layer as a form of regularization. After hyperparameter tuning, the best MLP parameters were identified to be 4 layers, with each layer containing 73, 71, 71, and 200 neurons respectively; a dropout probability of 0.1884; and learning rate of 1.4067×10⁻⁴. ResNet contains multiple blocks stacked over each other with ‘skip’ or ‘residual’ connections between blocks. Each block is a stack of two layers of neurons along with a layer of batch normalization and dropout. The output of each block is summed with its input and then passed on to the next block, thereby providing a ‘skip’ connection for information to flow. These ‘skip’ or ‘residual’ connections help in optimizing the training of deeper networks [1]. After hyperparameter tuning, the optimal parameters for the ResNet architecture were identified to be 6 blocks, with each block having two layers of 133 and 386 neurons respectively; a dropout probability of 0.2841; and learning rate of 1.3784×10⁻⁴.

Finally, the TabR architecture belongs to the family of retrieval-augmented neural networks. For a given target sample, TabR ‘retrieves’ a candidate set of samples from the training data that are most similar to the target sample and makes a final prediction using the information in the candidate set along with the target sample. The concept of retrieval-based models outside the realm of neural networks can be seen in methods like k-nearest neighbors [2]. To find similarity between samples, a single layer of neurons encodes the samples into a latent space and calculates the similarity between the latent representations. The encoded candidate samples and candidate labels are assigned weights (that sum to 1) based on their similarities to the target sample and summed with the encoded target sample. This is then passed through a final block of two layers of neurons, along with layer normalization and dropout, to obtain the final prediction. After hyperparameter tuning, the optimal model parameters were identified to be an encoded latent space of size 99; a dropout of 0.5385 for the candidate set weights; the final block layers with 198 and 99 neurons, along with dropout probabilities of 0.3497 and 0.0 after each layer; and a learning rate of 3.7944×10⁻⁵.

### Calculation of ProtAge

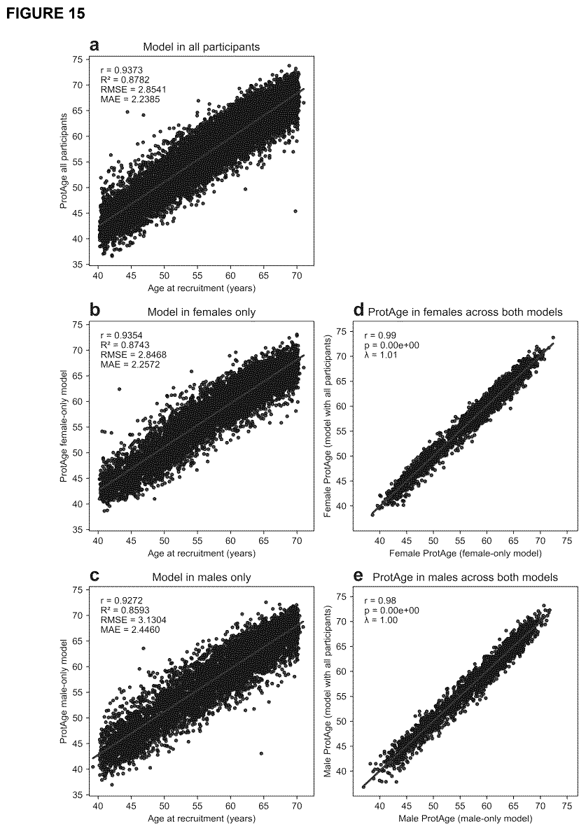

Using gradient boosting (LightGBM) as the selected model type, the inventors initially ran models trained separately on males and females, however the male- and female-only models showed similar age prediction performance to a model with both sexes (FIG. 15a-c) and protein predicted age from the sex-specific models were nearly perfectly correlated with protein predicted age from the model using both sexes (FIG. 15d-e). The inventors therefore calculated the proteomic age clock in both sexes combined to improve the generalizability of the findings.

To calculate proteomic age, the inventors first split all UKB participants (n=45,441) into 70/30 train/test splits. In the training data (n=31,808), the inventors trained a model to predict chronological age at recruitment using all 2,897 proteins in a single LightGBM model (Ke et al. 2017). First, model hyperparameters were tuned via 5-fold cross-validation using the Optuna module in Python (Akiba et al. 2019), with parameters tested across 200 trials and optimized to maximize the average R² of the models across all folds. The inventors then carried out Boruta feature selection via the shap-hypetune module. Boruta feature selection works by making random permutations of all features in the model (called shadow features), which are essentially random noise (Kursa et al. 2010). In the use of Boruta, at each iterative step these shadow features were generated and a model was run with all features and all shadow features. The inventors then removed all features that didn't have a mean of the absolute SHAP value that was higher than all random shadow features. The selection processes ended when there were no features remaining that didn't perform better than all shadow features. This procedure identified all relevant features to the outcome that have a greater influence on prediction than random noise. When running Boruta, the inventors used 200 trials and a threshold of 100% to compare shadow and real features (meaning that a real feature is selected if it performs better than 100% of shadow features). Third, the inventors re-tuned model hyperparameters for a new model with the subset of selected proteins using the same procedure as before. Both tuned LightGBM models before and after feature selection were checked for overfitting and validated by performing 5-fold cross-validation in the combined train set and testing the performance of the model against the holdout UKB test set. Across all analysis steps, LightGBM models were run with 5,000 estimators, 20 early stopping rounds, and using R² as a custom evaluation metric to identify the model that explained the maximum variation in age (according to R²).

Once the final model with Boruta-selected APs was trained in the UKB, the inventors calculated protein predicted age (ProtAge) for the entire UKB cohort (n=45,441) using 5-fold cross-validation. Within each fold, a LightGBM model was trained using the final hyperparameters and predicted age values were generated for the test set of that fold. The inventors then combined the predicted age values from each of the folds to create a measure of protein predicted age (ProtAge) for the entire sample. ProtAge was calculate in the CKB and FinnGen by using the trained UKB model to predict values in those datasets. Finally, the inventors calculated proteomic aging acceleration (ProtAgeAccel) separately in each cohort by taking the difference of ProtAge minus chronological age at recruitment separately in each cohort.

### Recursive Feature Elimination Using SHAP

For the recursive feature elimination analysis, the inventors started from the 204 Boruta-selected proteins. In each step, the inventors trained a model using 5-fold cross-validation in the UKB training data and then within each fold calculated the model R² and the contribution of each protein to the model as the mean of the absolute SHAP values across all participants for that protein. R² values were averaged across all 5 folds for each model. The inventors then removed the protein with the smallest mean of the absolute SHAP values and computed a new model, eliminating features recursively using this method until the inventors reached a model with only 5 proteins. If at any step of this process a different protein was identified as the least impactful in the different cross-validation folds, the inventors chose the protein ranked the lowest across the greatest number of folds to remove. The inventors identified 20 proteins as the smallest number of proteins that provide adequate prediction of chronological age. The inventors re-tuned hyperparameters for this 20-protein model (ProtAge20) using Optuna according to the methods described above, and the inventors also calculated proteomic age acceleration according to these top 20 proteins (ProtAgeAccel20) using 5-fold cross validation in the entire UKB cohort (45,441) using the methods described above.

### Bench Marking

All statistical benchmarking/utility analyses were carried out using Python v.3.6 and R v.4.2.2. All associations between ProtAgeAccel and aging biomarkers and physical/cognitive decline measures in the UKB were tested using linear/logistic regression using the statsmodels module (Skipper et al. 2010). All models were adjusted for age, sex, Townsend deprivation index, assessment center, self-reported ethnicity (Black, white, Asian, Mixed, Other), IPAQ activity group (low, moderate, high), and smoking status (never, previous, current). P-values were corrected for multiple comparisons via the False Discovery Rate (FDR) using the Benjamini-Hochberg method (Benjamini et al. 1995).

All associations between ProtAgeAccel and incident outcomes (mortality, 26 diseases) were tested using Cox proportional hazards models using the lifelines module (Davidson-Pilon 2023). Survival outcomes were defined using follow-up time to event and the binary incident event indicator. For all incident disease outcomes, prevalent cases were excluded from the dataset before models were run. For all incident outcome Cox modelling in the UKB, three successive models were tested with increasing numbers of covariates. Model 1 included adjustment for age at recruitment and sex. Model 2 included all model 1 covariates, plus Townsend deprivation index (Field ID 22189), assessment center (Field ID 54), physical activity (IPAQ activity group; Field ID 22032), and smoking status (Field ID 20116). Model 3 included all model 2 covariates plus BMI (Field ID 21001) and prevalent hypertension (definition in Table 24). P-values were corrected for multiple comparisons via FDR.

Functional enrichments (GO biological processes, GO molecular function, KEGG, Reactome) and protein-protein interaction (PPI) networks were downloaded from STRING (v.12) using the STRING API in Python. For functional enrichment analyses, the inventors used all proteins included in the Olink Explore 3072 platform as the statistical background (except for 19 Olink proteins that could not be mapped to STRING IDs. None of these proteins that could not be mapped were included in the final Boruta-selected proteins). The inventors only considered PPIs from STRING at a high level of confidence (>0.7) from the co-expression data.

SHAP interaction values from the trained LightGBM ProtAge model were retrieved using the shap module (Lundberg et al. 2010, Lundberg et al. 2017). SHAP-based PPI networks were generated by first taking the mean of the absolute value of each protein-protein SHAP interaction score across all samples. The inventors then used an interaction threshold of 0.0083 and removed all interactions below this threshold, which yielded a subset of variables similar in number to the node degree >2 threshold used for the STRING PPI network. Both SHAP-based and STRING-based (Szklarczyk et al. 2015) PPI networks were visualized and plotted using the NetworkX module (Hagberg et al. 2008).

Cumulative incidence curves and survival tables for deciles of ProtAgeAccel were calculated using KaplanMeierFitter from the lifelines module. Since the data were right-censored, the inventors plotted cumulative events against age at recruitment on the x-axis. All plots were generated using matplotlib (Hunter 2007) and seaborn (Waskom 2021).

### Example 2—Proteomic Age Clock

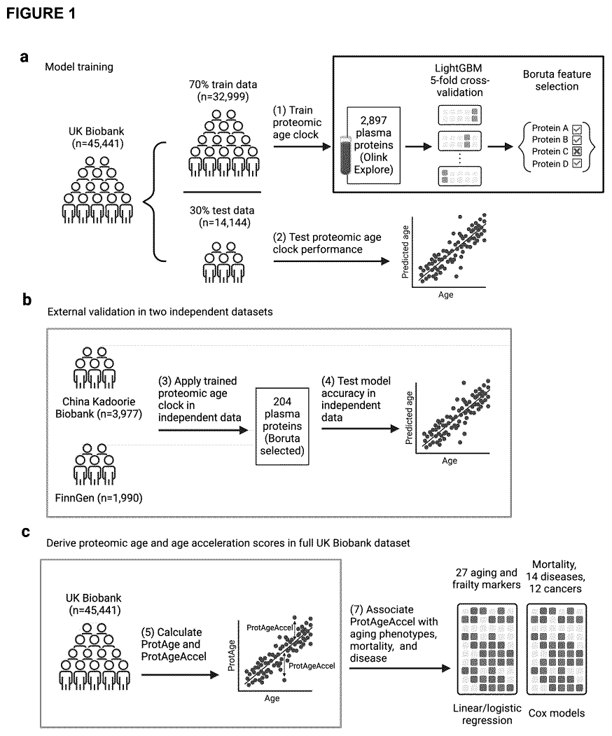

A schematic representation of the study design and main analytic approaches is shown in FIG. 1. Characteristics of participants across the discovery (UKB) and two validation cohorts are shown in Table 4. The inventors used plasma proteomic expression data from the subset of 45,441 randomly selected UKB participants (54% female, age range: 39-71 years), 3,977 Chinese (CKB) participants in an ischemic heart disease (IHD) case-cohort study (54% female, age range: 30-78 years), and 1,990 Finnish (FinnGen) participants (52% female, age range: 19-78 years). Across 11-16 years of follow-up in the UKB and 11-14 years of follow-up in the CKB, there were 4,828 (10.6%) and 1,426 (36%) deaths, respectively. Proteomic profiling was conducted among mostly healthy participants in FinnGen without major diseases and only 1% (n=22) died during follow up.

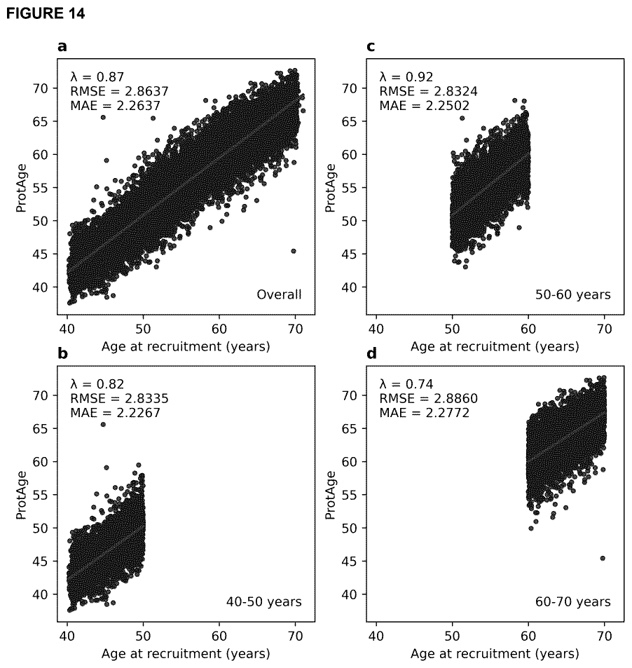

The inventors randomly split the UKB cohort into 70% training and 30% test sets to develop the proteomic age clock. In the training phase, the inventors compared six machine learning methods (LASSO, elastic net, gradient boosting, and three neural networks) to train proteomic age clock models to predict chronological age using normalized expression of 2,897 proteins from the Olink Explore 3027 panel. The inventors found that gradient boosting (LightGBM, Ke et al 2017) showed the second best age prediction accuracy in the UKB test set (n=13,633) and the highest accuracy in the independent samples from the CKB and FinnGen (FIG. 12). After selecting LightGBM as the final model, the inventors used the Boruta feature selection algorithm (Kursa et al. 2010) and SHAP values (SHapley Additive exPlanations, Lundberg et al. 2020) to identify the subset of all proteins relevant for predicting chronological age (see Example 1). This process resulted in the identification of 204 APs in the dataset (Tables 2 and 5). Protein predicted age (ProtAge) from this 204-protein model explained a similar degree of variation in chronological age compared with the 2,897-protein model (FIG. 13a-b), with similar model error across different age groups (FIG. 14). The gradient boosting ProtAge model explained a high degree of variation in chronological age in the UKB test set (R²=0.88; Pearson r=0.94) and the independent validation sets from the CKB (R²=0.85; Pearson r=0.92) and FinnGen (R²=0.86; Pearson r=0.94) (FIG. 2d-f).

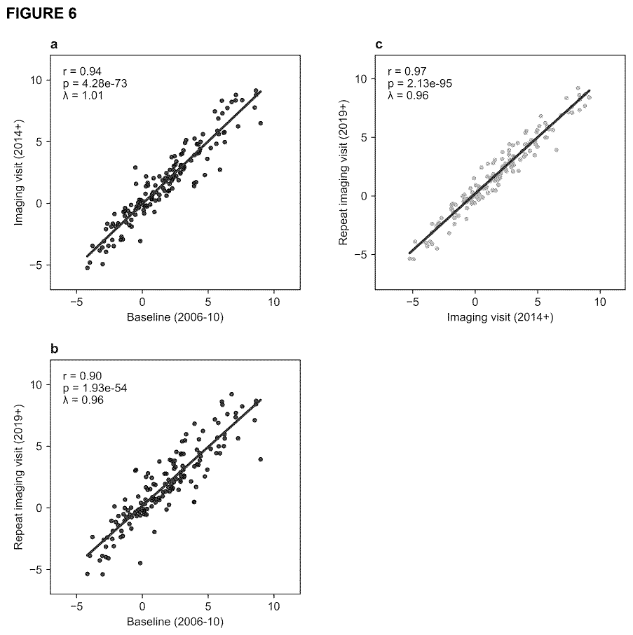

To assess whether each of the AP's association with age was stable over time, the inventors used repeat protein expression measurements available for a subset of 149 proteins in the model among 1,085 UKB participants who had proteomic data measured at three time points (baseline [2006-11], imaging study visit [2014+], and the repeat imaging visit [2019+]). For each of these 149 APs, the inventors assessed their association with age at each study visit using linear regression. Beta coefficients for the associations of these APs with age across all three time points were strongly correlated with each other (Pearson r=0.89-0.97), suggesting good stability of associations between APs and age across repeat visits spanning at least 9-13 years (FIG. 6).

Using 204 APs in the final model, the inventors calculated accelerated proteomic aging (ProtAgeAccel) as the difference between ProtAge and chronological age in all three cohorts. In the UKB, the average years of biological age acceleration among the top 5% and bottom 5% of ProtAgeAccel was 6.3 and −6 years, respectively, resulting in a mean difference of approximately 12.3 years in biological aging between them. ProtAgeAccel showed similar distributions across all three cohorts in females and males, across self-reported ethnicities in the UKB, and across geographical regions in the CKB (FIG. 2g-i).

As a final feature selection step, the inventors explored whether recursive feature elimination using SHAP values could identify a much smaller set of proteins (<50) that accurately predict chronological age (see Methods). The inventors identified a model of 20 proteins (ProtAge20) that achieved 91% of the age prediction performance of the 204-protein model (R²=0.78, Pearson r=0.89; FIG. 13c-d; Tables 1 and 6). The inventors further calculated accelerated proteomic aging according to these top 20 proteins (ProtAgeAccel20) in the UKB, using the same approach as above.

### Example 3—Proteomic Aging Predicts Frailty and Aging Phenotypes

To understand how accelerated proteomic aging may influence aging-related physiological and cognitive status, the inventors examined the associations in the UKB of ProtAgeAccel with: (i) a comprehensive frailty index (Williams et al. 2019, see Example 1); (ii) 16 individual measures of physical (e.g., slow walking pace, grip strength) and cognitive status (reaction time, fluid intelligence), and (iii) 10 measures of biological aging (e.g., telomere length, insulin-like growth factor 1 [IGF-1]) and clinical blood biochemistry (e.g., albumin, creatinine). After adjustment for chronological age, sex, and major sociodemographic and lifestyle confounders, ProtAgeAccel was significantly associated with all measures investigated except for two liver biomarkers (alanine aminotransferase [ALT] and total bilirubin; FIG. 3a-b). Among biological aging mechanisms investigated (FIG. 3a), increasing ProtAgeAccel was associated with increasing levels of two kidney function biomarkers (Cystatin C, Creatinine), two liver enzymes (aspartate aminotransferase [AST], gamma-glutamyl transferase [GGT]), and C-reactive protein; and was associated with decreased levels of albumin, IGF-1, and telomere length. Among physical measures (FIG. 3b), increasing ProtAgeAccel was associated with poor self-rated health, slow walking pace, self-rating one's face as older than average, sleeping 210 hours per day, feeling tired every day, and having frequent insomnia. It was also associated with higher values of a frailty index, systolic and diastolic blood pressure, longer (slower) reaction time, arterial stiffness, and BMI; and with lower values of bone mineral density, fluid intelligence, lung function, and hand grip strength.

To explore whether these associations are explained by reverse causation (i.e., resulting from a non-detected pathology), the inventors restricted the analyses to a subset of UKB participants who had no lifetime diagnoses (according to hospital inpatient, cancer registry, and GP records) of any of the 26 diseases studied (n=20,353). Among these participants (FIG. 3c-d), the inventors found that ProtAgeAccel remained significantly associated with nearly all markers except for albumin (which is a typical protein marker of end-stage morbidity), self-rated facial aging, sleeping for 10+ hours/day, and feeling tired every day (FIG. 3d).

ProtAgeAccel20 was also associated with all aging functional phenotypes except for diastolic blood pressure (DBP). Compared with the 204-protein model, ProtAgeAccel20 showed stronger effect estimates in relation to biological measures of aging (e.g., telomeres, IGF-1) (FIG. 3a) but somewhat smaller effect estimates for measures of frailty and physiological/cognitive decline (FIG. 3b). ProtAgeAccel20 was significantly associated with all biological aging markers (FIG. 3c) in the subset of UKB participants without lifetime disease diagnoses, and was associated with all physiological measures except sleeping for 10+ hours/day, DBP, and BMI (FIG. 3d).

Summary statistics from all models are shown in Tables 7-10.

### Example 4—Proteomic Age Acceleration is a Strong Predictor of Common Diseases

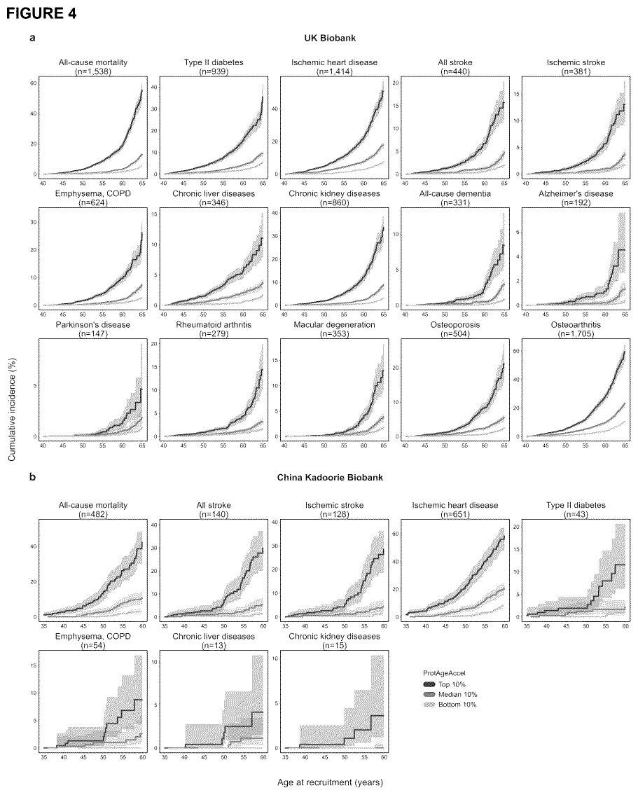

UKB participants in the top, median, and bottom deciles of ProtAgeAccel showed divergent age-specific incidence rates of all-cause mortality and the 14 common non-cancer diseases studied (FIG. 4a; Table 20). Cumulative incidence risk trajectories according to these deciles of ProtAgeAccel were similar in females and males. For those aged 65 years at recruitment, the highest cumulative incident rates (equivalent to absolute risk) across the study follow-up period of 11-16 years for the top decile of ProtAgeAccel were observed for osteoarthritis (59.4%), all-cause mortality (55.2%), IHD (50.6%) type 2 diabetes (T2D; 35.3%), and chronic kidney disease (CKD; 33.6%). Neurodegenerative diseases (Parkinson's disease, all-cause dementia, Alzheimer's disease [AD]) all showed cumulative incidence rates below 1% in the bottom decile of ProtAgeAccel across all recruitment ages.

In the CKB, the inventors also calculated cumulative incidence rates according to deciles of ProtAgeAccel for diseases with >10 incident cases across the 3 deciles of ProtAgeAccel (FIG. 4b; Table 21). The inventors observed significant differences for IHD, all-cause mortality, all stroke, and ischemic stroke. Differences were also observed for T2D, chronic obstructive pulmonary disease (COPD), chronic liver diseases, and CKD, however confidence intervals were much wider due to a smaller number of incident cases.

The inventors further used multivariable Cox proportional hazards models to investigate whether associations of ProtAgeAccel with mortality and the 14 common diseases persisted after adjustment for chronological age, sex, smoking, physical activity, sociodemographic factors, and clinical risk factors. ProtAgeAccel showed a significant association with mortality and all non-cancer incident disease outcomes except Parkinson's disease across all models in the UKB (FIG. 5). In the fully adjusted model that also included covariates for BMI and prevalent hypertension (Model 3), the largest effect size per one year increase of ProtAgeAccel were observed for AD (HR: 1.15; 95% Cl: 1.12-1.19), all-cause dementia (HR: 1.13; 95% Cl: 1.1-1.16) and CKD (HR: 1.10; 95% Cl: 1.08-1.11). ProtAgeAccel20 was associated with all diseases investigated, including Parkinson's. Summary statistics from all models are shown in Tables 11-16.

Based on the HR per year increase of ProtAgeAccel for each outcome shown above, the inventors estimated that those in the top 5% of ProtAgeAccel had on average a 2.5-fold higher risk of AD than those with no difference between ProtAge and chronological age (HR of 1.156.3=2.6), and a 5.8-fold higher risk of AD (HR of 1.15(6.3+[−6])) compared with those in the bottom 5% of biological age acceleration. For CKD, the increases in risk were 1.8-fold (top 5% vs. 0) and 3.1-fold (top 5% vs. bottom 5%), and for mortality the increases in risk are 1.9-fold (top 5% vs. 0) and 3.6-fold (top 5% vs. bottom 5%).

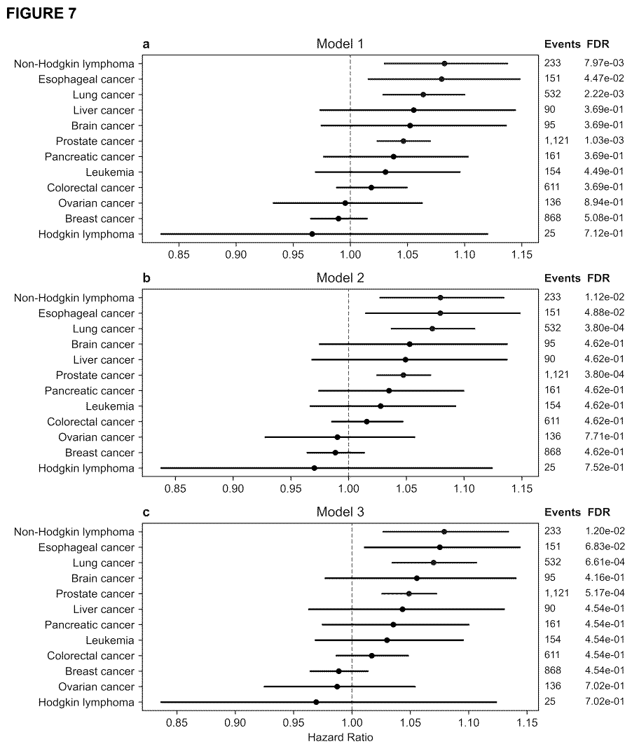

In Cox multivariable models, ProtAgeAccel was associated with only four cancers (esophageal, lung, non-Hodgkin lymphoma, and prostate) after adjustment for age, sex, sociodemographic and lifestyle factors, BMI, and prevalent hypertension (FIG. 7). Summary statistics are shown in Tables 17-19.

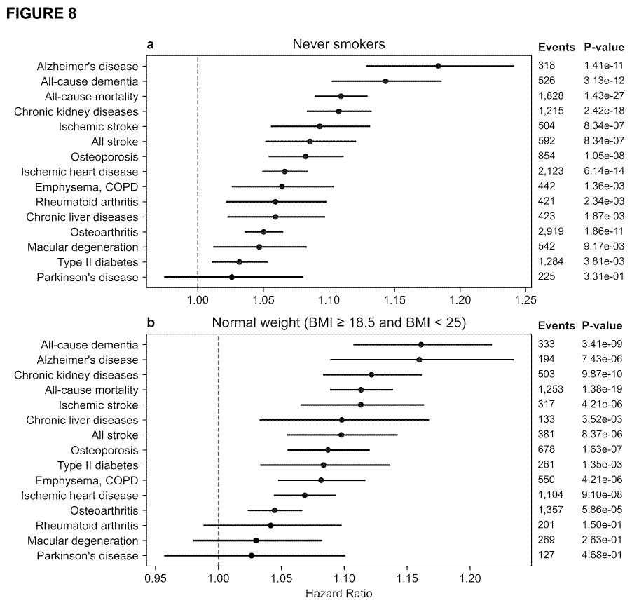

Although the analyses described above were adjusted for smoking status, the inventors conducted further sensitivity analyses in never smokers. Among never smokers, ProtAgeAccel remained significantly associated with mortality and all non-cancer outcomes except Parkinson's disease (FIG. 8a). In a similar sensitivity analysis restricted to those within a normal weight range (BMI≥18.5 & BMI<25), ProtAgeAccel remained significantly associated with all outcomes except Parkinson's disease, macular degeneration, and rheumatoid arthritis (FIG. 8b).

### Example 5—Proteomic Age Acceleration Increases with Increasing Multimorbidity

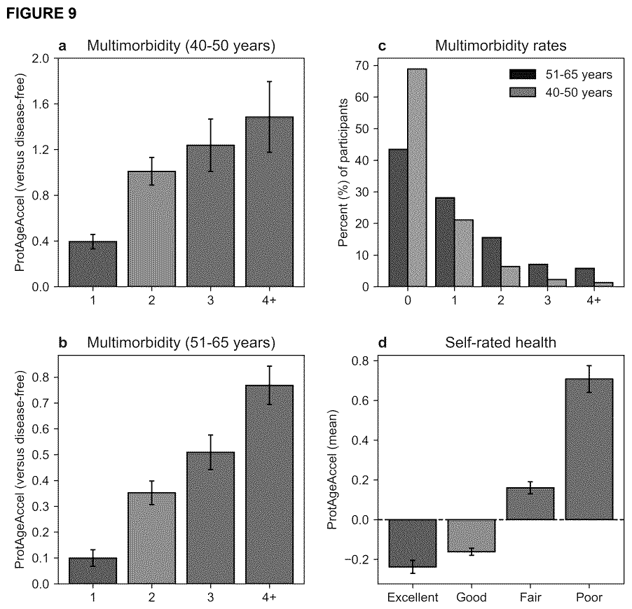

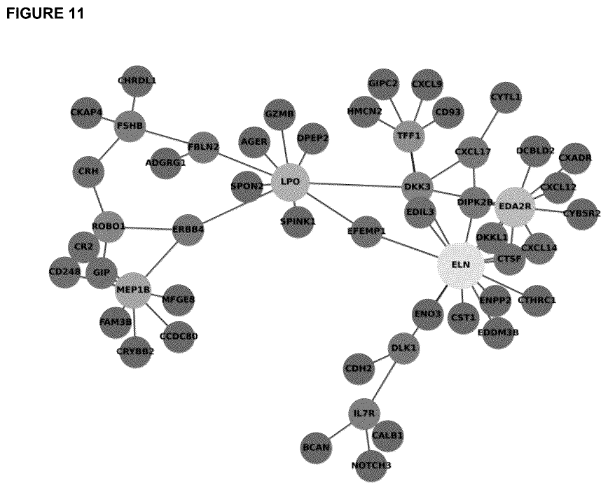

The inventors defined multimorbidity as the number of lifetime diagnoses of any of the 26 diseases examined in the UKB, and categorized participants according to having 0, 1, 2, 3, or 4+ lifetime diagnoses. The inventors found that the average years of ProtAgeAccel increased with number of lifetime conditions (FIG. 9). The inventors also found that this effect was more pronounced for younger participants at recruitment (aged 40-50 years; FIG. 11a), among whom presence of disease was less common (FIG. 9c). On average, 1.5 greater years of ProtAgeAccel was observed in those with 4+ lifetime diagnoses compared to those with 0 diagnoses in participants aged 40-50 years at recruitment (FIG. 9a), whereas in those aged 51-65 years at recruitment the inventors observed 0.8 greater years of ProtAgeAccel (FIG. 9b). The relationship between ProtAgeAccel and multimorbidity status derived from health records was also reflected in self-reported health information. On average, 0.9 fewer years of ProtAgeAccel was observed in those reporting excellent health (likely no diseases present) compared with those reporting poor self-reported health (FIG. 9d).

### Example 6—Biological Functions and Protein-Protein Interaction Networks Among Aging Proteins

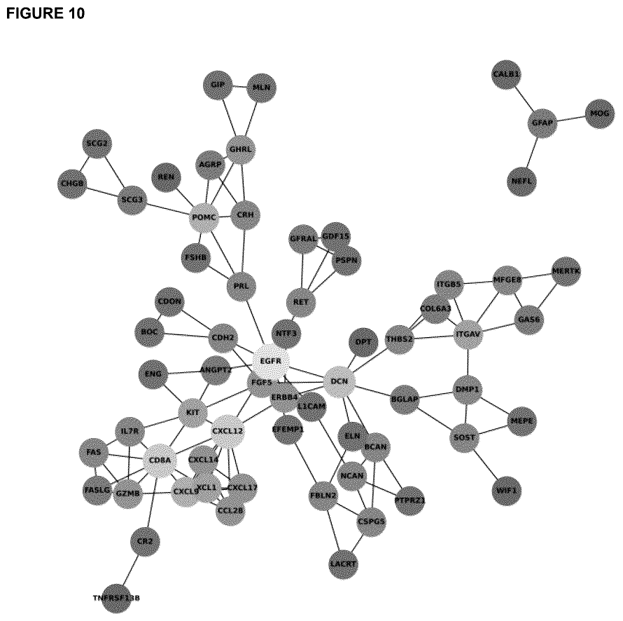

Testing for functional enrichment among the 204 APs revealed that these APs were enriched for one Gene Ontology (GO) biological processes: anatomical structure development and developmental process. No enrichments were found using GO molecular function, Kyoto Encyclopedia of Genes and Genomes (KEGG), or Reactome. However, these 204 APs showed highly interconnected subnetwork of 66 proteins with at least 2 node connections in a PPI network using co-expression information from the STRING database (FIG. 10).

Individual proteins with the greatest numbers of connections to other proteins were EGFR (involved in cancer drug resistance, brain structure, and platelet count), CXCL12 (an immune-related chemokine involved in immune surveillance, inflammation response, tissue homeostasis, and tumor growth and metastasis), ITGAV (an integrin protein implicated in body height, handedness, dyslexia, and albumin/creatinine metabolism), CXCL9 (implicated in T-cell function and inflammation), and CD8A (a CD8 antigen implicated in the innate immune system).

The inventors also used SHAP interaction values from the trained ProtAge model to calculate a second PPI network that represents the interactions of proteins together in the model to predict age (FIG. 11). Individual proteins with the largest numbers of connections to other proteins according to SHAP interaction values were ELN (an elastic fiber protein that makes up part of the extracellular matrix and confers elasticity to organs and tissues including the heart, skin, lungs, ligaments, and blood vessels), EDA2R (involved in the NF-κB and innate immune pathways and implicated in baldness, estradiol, testosterone and HDL metabolism), LTPB2 (a protein involved in BMI, blood pressure, neuroticism and anxiety, glaucoma and retina pathology, lung function and mortality), CXCL17 (a chemokine interacting with CXCL9, that plays a role in tumor genesis, antimicrobial defense through monocytes, macrophages, and dendritic cells), and GDF15 (implicated in BMI, liver function, systemic lupus erythematosus, and COVID-19). Overall, the inventors found quite distinct results when using a data driven approach to modelling PPIs using interactions from the machine learning models versus using the most up-to-date experimental biological knowledge from the STRING database.

The inventors further examined the roles and functions of the 20 proteins comprising the ProtAge20 score, which together capture ˜91% of the 204-protein model's ability to predict age. These key APs are involved in: (1) cell adhesion and extracellular matrix (ECM) interactions (ELN, COL6A3, CDCP1, PODXL2, LTBP2, SCARF2, ENG); (2) immune response and inflammation (CXCL17, LECT2, SCARF2, GDF15); (3) hormone regulation and reproduction (FSHB, AGRP, ACRV1); (4) cell signalling (EDA2R, SCARF2, PTPRR); (5) protease activity and enzymatic function (KLK3, KLK7:); (6) regulation of body weight and energy balance (GDF15, AGRP); (7) neuronal structure and function (GFAP, NEFL), and (8) development and differentiation (EDA2R, LTBP2, ENG).

### Tables

TABLE 1
20 biomarker panel

| | |
|---|---|
| Acrosomal protein SP-10 | Glial fibrillary acidic protein |
| Agouti-related protein | Immunoglobulin superfamily DCC subclass |
| | member 4 |
| CUB domain-containing protein 1 | Prostate-specific antigen |
| Collagen alpha-3(VI) chain | Kallikrein-7 |
| C-X-C motif chemokine 17 | Leukocyte cell-derived chemotaxin-2 |
| Tumor necrosis factor receptor superfamily | Latent-transforming growth factor beta- |
| member 27 | binding protein 2 |
| Elastin | Neurofilament light polypeptide |
| Endoglin | Podocalyxin-like protein 2 |
| Follitropin subunit beta | Receptor-type tyrosine-protein phosphatase |
| | R |
| Growth/differentiation factor 15 | Scavenger receptor class F member 2 |

TABLE 2
204 biomarker panel

| | |
|---|---|
| Acrosomal protein SP-10 | PDZ domain-containing protein GIPC2 |
| Actin, aortic smooth muscle | Pancreatic secretory granule membrane |
| | major glycoprotein GP2 |
| Adenosine deaminase | Granzyme B |
| A disintegrin and metalloproteinase with | Hepatitis A virus cellular receptor 1 |
| thrombospondin motifs 13 | |
| A disintegrin and metalloproteinase with | Hemicentin-2 |
| thrombospondin motifs 15 | |
| A disintegrin and metalloproteinase with | Corticosteroid 11-beta-dehydrogenase |
| thrombospondin motifs 16 | isozyme 1 |
| ADAMTS-like protein 5 | Immunoglobulin superfamily DCC subclass |
| | member 4 |
| Adhesion G-protein coupled receptor G1 | Interleukin-17D |
| Alpha-fetoprotein | Interleukin-5 receptor subunit alpha |
| Advanced glycosylation end product- | Interleukin-7 receptor subunit alpha |
| specific receptor | |
| Agouti-related protein | Insulin-like 3 |
| Protein AHNAK2 | Integrin alpha-V |
| Angiopoietin-2 | Integrin beta-5 |
| BAG family molecular chaperone | Integrin beta-like protein 1 |
| regulator 3 | |
| Brevican core protein | Kinesin-like protein KIF22 |
| Osteocalcin | Mast/stem cell growth factor receptor Kit |
| Brother of CDO | Kallikrein-14 |
| Basigin | Prostate-specific antigen |
| Protein C19orf12 | Kallikrein-4 |
| Complement C1q-like protein 2 | Kallikrein-7 |
| Carbonic anhydrase 14 | Kallikrein-8 |
| Carbonic anhydrase 4 | Killer cell lectin-like receptor subfamily F |
| | member 1 |
| Calbindin | Neural cell adhesion molecule L1 |
| Coiled-coil domain-containing protein 80 | Extracellular glycoprotein lacritin |
| C-C motif chemokine 28 | Leukocyte cell-derived chemotaxin-2 |
| CCN family member 5 | Protein LEG1 homolog |
| T-cell surface glycoprotein CD1c | Lutropin subunit beta |
| Endosialin | Leiomodin-1 |
| T-cell surface glycoprotein CD8 alpha | Lactoperoxidase |
| chain | |
| Complement component C1q receptor | Latent-transforming growth factor beta- |
| | binding protein 2 |
| CUB domain-containing protein 1 | Ly6/PLAUR domain-containing protein 3 |
| Cadherin-2 | Apical endosomal glycoprotein |
| Cadherin-3 | Matrilin-3 |
| Cadherin-related family member 2 | Meprin A subunit beta |
| Cell adhesion molecule-related/down- | Matrix extracellular phosphoglycoprotein |
| regulated by oncogenes | |
| Cadherin EGF LAG seven-pass G-type | Tyrosine-protein kinase Mer |
| receptor 2 | |
| Complement factor H-related protein 5 | Lactadherin |
| Secretogranin-1 | Promotilin |
| Chitotriosidase-1 | Macrophage metalloelastase |
| Chordin-like protein 1 | Myelin-oligodendrocyte glycoprotein |
| Chordin-like protein 2 | Matrix remodeling-associated protein 8 |
| Cytoskeleton-associated protein 4 | Neurocan core protein |
| C-type lectin domain family 14 member A | Neurofilament light polypeptide |
| Contactin-5 | Nucleoside diphosphate kinase 3 |
| Collagen alpha-1(XV) chain | Neurogenic locus notch homolog protein 3 |
| Collagen alpha-3(VI) chain | N-acetylneuraminate lyase |
| Collagen alpha-1(IX) chain | Neuronal pentraxin-2 |
| Complement receptor type 2 | Neurotrophin-3 |
| Corticoliberin | Neurotrophin-4 |
| Cartilage acidic protein 1 | N-terminal prohormone of brain natriuretic |
| | peptide |
| Beta-crystallin B2 | Odontogenic ameloblast-associated protein |
| Chondroitin sulfate proteoglycan 5 | Glycodelin |
| Cystatin-SN | Inactive serine protease PAMR1 |
| Cystatin-D | phospholipase A2 inhibitor and Ly6/PLAUR |
| | domain-containing protein |
| Collagen triple helix repeat-containing | Polycystin-1 |
| protein 1 | |
| Cathepsin F | Tissue-type plasminogen activator |
| Cathepsin L2 | Podocalyxin-like protein 2 |
| Coxsackievirus and adenovirus receptor | Pro-opiomelanocortin |
| Stromal cell-derived factor 1 | Prolargin |
| C-X-C motif chemokine 14 | Prolactin |
| C-X-C motif chemokine 17 | Prion-like protein doppel |
| C-X-C motif chemokine 9 | Prokineticin-1 |
| NADH-cytochrome b5 reductase 2 | Persephin |
| Cytokine-like protein 1 | Prostaglandin-H2 D-isomerase |
| Discoidin, CUB and LCCL domain- | Pleiotrophin |
| containing protein 2 | |
| Decorin | Receptor-type tyrosine-protein phosphatase |
| | mu |
| Divergent protein kinase domain 2B | Receptor-type tyrosine-protein phosphatase |
| | N2 |
| Dickkopf-related protein 3 | Receptor-type tyrosine-protein phosphatase |
| | R |
| Dickkopf-like protein 1 | Receptor-type tyrosine-protein phosphatase |
| | zeta |
| Protein delta homolog 1 | Renin |
| Dentin matrix acidic phosphoprotein 1 | Proto-oncogene tyrosine-protein kinase |
| | receptor Ret |
| Dipeptidase 2 | Repulsive guidance molecule A |
| Dermatopontin | RGM domain family member B |
| Tumor necrosis factor receptor | Prorelaxin H2 |
| superfamily member 27 | |
| Epididymal secretory protein E3-beta | Roundabout homolog 1 |
| EGF-like repeat and discoidin I-like | Ribonucleoside-diphosphate reductase |
| domain-containing protein 3 | subunit M2 |
| EGF-containing fibulin-like extracellular | Scavenger receptor class F member 2 |
| matrix protein 1 | |
| EF-hand domain-containing protein D1 | Secretogranin-2 |
| Epidermal growth factor receptor | Secretogranin-3 |
| Elastin | Uteroglobin |
| Protein enabled homolog | Protein sidekick-2 |
| Endoglin | Neuronal-specific septin-3 |
| Beta-enolase | Superoxide dismutase [Mn], mitochondrial |
| Ectonucleotide | VPS10 domain-containing receptor SorCS2 |
| pyrophosphatase/phosphodiesterase | |
| family member 2 | |
| Ectonucleotide | Sclerostin |
| pyrophosphatase/phosphodiesterase | |
| family member 5 | |
| Receptor tyrosine-protein kinase erbB-4 | Serine protease inhibitor Kazal-type 1 |
| Fatty acid-binding protein, adipocyte | Spondin-2 |
| Protein FAM3B | Small proline-rich protein 3 |
| Prolyl endopeptidase FAP | Sushi repeat-containing protein SRPX |
| Tumor necrosis factor receptor | Sushi domain-containing protein 2 |
| superfamily member 6 | |
| Tumor necrosis factor ligand superfamily | Sushi domain-containing protein 5 |
| member 6 | |
| Fibulin-2 | Trefoil factor 1 |
| Fc receptor-like protein 2 | Thrombospondin-2 |
| Fibroblast growth factor 5 | Tumor necrosis factor receptor superfamily |
| | member 11B |
| Follitropin subunit beta | Tumor necrosis factor receptor superfamily |
| | member 13B |
| Follistatin-related protein 1 | Tumor necrosis factor ligand superfamily |
| | member 13 |
| Growth arrest-specific protein 6 | Tenascin-X |
| Growth/differentiation factor 15 | Tetraspanin-1 |
| Glial fibrillary acidic protein | WAP four-disulfide core domain protein 2 |
| GDNF family receptor alpha-like | Wnt inhibitory factor 1 |
| Appetite-regulating hormone | Protein Wnt-9a |
| Gastric inhibitory polypeptide | Lymphotactin |

TABLE 3
Table 3. 10 biomarker panel

| | |
|---|---|
| Tumor necrosis factor receptor | Elastin |
| superfamily member 27 | |
| Collagen alpha-3(VI) chain | Immunoglobulin superfamily DCC |
| | subclass member 4 |
| Growth/differentiation factor 15 | Follitropin subunit beta |
| Neurofilament light polypeptide | Latent-transforming growth factor beta- |
| | binding protein 2 |
| Podocalyxin-like protein 2 | Prostate-specific antigen |

TABLE 4
Characteristics of study participants across three cohorts.
CKB: China Kadoorie Biobank; COPD: Chronic obstructive pulmonary
disease; IHD: Ischemic heart disease; UKB: UK Biobank

| | UKB | | CKB | | FinnGen | |
|---|---|---|---|---|---|---|
| | (N = 45,441) | | (N = 3,977) | | (N = 1,990) | |
| Age | | | | | | |
| Mean (SD) | 57 | (8.2) | 57 | (12) | 56 | (15) |
| Range (years) | 39-71 | | 30-78 | | 19-78 | |
| Sex | | | | | | |
| Female | 24,579 | (54.1%) | 2,137 | (53.7%) | 1,032 | (51.9%) |
| BMI (kg/m2) | | | | | | |
| Mean (SD) | 27 | (4.8) | 24 | (3.6) | 26 | (4.5) |
| Ethnicity | | | | | | |
| White | 42,320 | (93.1%) | — | | — | |
| Asian | 1,016 | (2.2%) | — | | — | |
| Black | 1,114 | (2.5%) | — | | — | |
| Mixed | 293 | (0.6%) | — | | — | |
| Other | 554 | (1.2%) | — | | — | |
| Geographic region | | | | | | |
| Gansu (Rural) | — | | 397 | (10.0%) | — | |
| Haikou (Urban) | — | | 298 | (7.5%) | — | |
| Harbin (Urban) | — | | 598 | (15.0%) | — | |
| Henan (Rural) | — | | 493 | (12.4%) | — | |
| Hunan (Rural) | — | | 462 | (11.6%) | — | |
| Liuzhou (Urban) | — | | 379 | (9.5%) | — | |
| Qingdao (Urban) | — | | 415 | (10.4%) | — | |
| Sichuan (Rural) | — | | 341 | (8.6%) | — | |
| Suzhou (Urban) | — | | 252 | (6.3%) | — | |
| Zhejiang (Rural) | — | | 342 | (8.6%) | — | |
| Incident diabetes | | | | | | |
| Yes | 2,781 | (6.1%) | 2,781 | (6.1%) | — | |
| Incident IHD | | | | | | |
| Yes | 4,546 | (10.0%) | 4,546 | (10.0%) | — | |
| Incident all stroke | | | | | | |
| Yes | 1,362 | (3.0%) | 1,362 | (3.0%) | — | |
| Incident all stroke | | | | | | |
| Yes | 1,182 | (2.6%) | 1,182 | (2.6%) | — | |
| Incident COPD | | | | | | |
| Yes | 2,059 | (4.5%) | 2,059 | (4.5%) | — | |
| Incident chronic liver diseases | | | | | | |
| Yes | 1,011 | (2.2%) | 1,011 | (2.2%) | — | |
| Incident chronic kidney diseases | | | | | | |
| Yes | 2,626 | (5.8%) | 2,626 | (5.8%) | — | |
| All-cause mortality | | | | | | |
| Dead | 4,828 | (10.6%) | 4,828 | (10.6%) | 22 | (1.1%) |

TABLE 5
Biomarkers significant in ProtAge model. A list of
all 204 biomarkers identified in the aging model.
Further included are the UniProt ID for each protein.

| Gene name | Protein name | UniProt ID |
|---|---|---|
| ACRV1 | Acrosomal protein SP-10 | P26436 |
| ACTA2 | Actin, aortic smooth muscle | P62736 |
| ADA | Adenosine deaminase | P00813 |
| ADAMTS13 | A disintegrin and | Q76LX8 |
| | metalloproteinase with | |
| | thrombospondin motifs 13 | |
| ADAMTS15 | A disintegrin and | Q8TE58 |
| | metalloproteinase with | |
| | thrombospondin motifs 15 | |
| ADAMTS16 | A disintegrin and | Q8TE57 |
| | metalloproteinase with | |
| | thrombospondin motifs 16 | |
| ADAMTSL5 | ADAMTS-like protein 5 | Q6ZMM2 |
| ADGRG1 | Adhesion G-protein coupled | Q9Y653 |
| | receptor G1 | |
| AFP | Alpha-fetoprotein | P02771 |
| AGER | Advanced glycosylation end | Q15109 |
| | product-specific receptor | |
| AGRP | Agouti-related protein | O00253 |
| AHNAK2 | Protein AHNAK2 | Q8IVF2 |
| ANGPT2 | Angiopoietin-2 | O15123 |
| BAG3 | BAG family molecular chaperone | O95817 |
| | regulator 3 | |
| BCAN | Brevican core protein | Q96GW7 |
| BGLAP | Osteocalcin | P02818 |
| BOC | Brother of CDO | Q9BWV1 |
| BSG | Basigin | P35613 |
| C19orf12 | Protein C19orf12 | Q9NSK7 |
| C1QL2 | Complement C1q-like protein 2 | Q7Z5L3 |
| CA14 | Carbonic anhydrase 14 | Q9ULX7 |
| CA4 | Carbonic anhydrase 4 | P22748 |
| CALB1 | Calbindin | P05937 |
| CCDC80 | Coiled-coil domain-containing | Q76M96 |
| | protein 80 | |
| CCL28 | C-C motif chemokine 28 | Q9NRJ3 |
| CCN5 | CCN family member 5 | O76076 |
| CD1C | T-cell surface glycoprotein CD1c | P29017 |
| CD248 | Endosialin | Q9HCU0 |
| CD8A | T-cell surface glycoprotein CD8 | P01732 |
| | alpha chain | |
| CD93 | Complement component C1q | Q9NPY3 |
| | receptor | |
| CDCP1 | CUB domain-containing protein 1 | Q9H5V8 |
| CDH2 | Cadherin-2 | P19022 |
| CDH3 | Cadherin-3 | P22223 |
| CDHR2 | Cadherin-related family member 2 | Q9BYE9 |
| CDON | Cell adhesion molecule- | Q4KMG0 |
| | related/down-regulated by | |
| | oncogenes | |
| CELSR2 | Cadherin EGF LAG seven-pass | Q9HCU4 |
| | G-type receptor 2 | |
| CFHR5 | Complement factor H-related | Q9BXR6 |
| | protein 5 | |
| CHGB | Secretogranin-1 | P05060 |
| CHIT1 | Chitotriosidase-1 | Q13231 |
| CHRDL1 | Chordin-like protein 1 | Q9BU40 |
| CHRDL2 | Chordin-like protein 2 | Q6WN34 |
| CKAP4 | Cytoskeleton-associated protein 4 | Q07065 |
| CLEC14A | C-type lectin domain family 14 | Q86T13 |
| | member A | |
| CNTN5 | Contactin-5 | O94779 |
| COL15A1 | Collagen alpha-1(XV) chain | P39059 |
| COL6A3 | Collagen alpha-3(VI) chain | P12111 |
| COL9A1 | Collagen alpha-1(IX) chain | P20849 |
| CR2 | Complement receptor type 2 | P20023 |
| CRH | Corticoliberin | P06850 |
| CRTAC1 | Cartilage acidic protein 1 | Q9NQ79 |
| CRYBB2 | Beta-crystallin B2 | P43320 |
| CSPG5 | Chondroitin sulfate proteoglycan 5 | O95196 |
| CST1 | Cystatin-SN | P01037 |
| CST5 | Cystatin-D | P28325 |
| CTHRC1 | Collagen triple helix repeat- | Q96CG8 |
| | containing protein 1 | |
| CTSF | Cathepsin F | Q9UBX1 |
| CTSV | Cathepsin L2 | O60911 |
| CXADR | Coxsackievirus and adenovirus | P78310 |
| | receptor | |
| CXCL12 | Stromal cell-derived factor 1 | P48061 |
| CXCL14 | C-X-C motif chemokine 14 | O95715 |
| CXCL17 | C-X-C motif chemokine 17 | Q6UXB2 |
| CXCL9 | C-X-C motif chemokine 9 | Q07325 |
| CYB5R2 | NADH-cytochrome b5 reductase 2 | Q6BCY4 |
| CYTL1 | Cytokine-like protein 1 | Q9NRR1 |
| DCBLD2 | Discoidin, CUB and LCCL domain- | Q96PD2 |
| | containing protein 2 | |
| DCN | Decorin | P07585 |
| DIPK2B | Divergent protein kinase domain | Q9H7Y0 |
| | 2B | |
| DKK3 | Dickkopf-related protein 3 | Q9UBP4 |
| DKKL1 | Dickkopf-like protein 1 | Q9UK85 |
| DLK1 | Protein delta homolog 1 | P80370 |
| DMP1 | Dentin matrix acidic | Q13316 |
| | phosphoprotein 1 | |
| DPEP2 | Dipeptidase 2 | Q9H4A9 |
| DPT | Dermatopontin | Q07507 |
| EDA2R | Tumor necrosis factor receptor | Q9HAV5 |
| | superfamily member 27 | |
| EDDM3B | Epididymal secretory protein E3- | P56851 |
| | beta | |
| EDIL3 | EGF-like repeat and discoidin I- | O43854 |
| | like domain-containing protein 3 | |
| EFEMP1 | EGF-containing fibulin-like | Q12805 |
| | extracellular matrix protein 1 | |
| EFHD1 | EF-hand domain-containing | Q9BUP0 |
| | protein D1 | |
| EGFR | Epidermal growth factor receptor | P00533 |
| ELN | Elastin | P15502 |
| ENAH | Protein enabled homolog | Q8N8S7 |
| ENG | Endoglin | P17813 |
| ENO3 | Beta-enolase | P13929 |
| ENPP2 | Ectonucleotide | Q13822 |
| | pyrophosphatase/phosphodiesterase | |
| | family member 2 | |
| ENPP5 | Ectonucleotide | Q9UJA9 |
| | pyrophosphatase/phosphodiesterase | |
| | family member 5 | |
| ERBB4 | Receptor tyrosine-protein kinase | Q15303 |
| | erbB-4 | |
| FABP4 | Fatty acid-binding protein, | P15090 |
| | adipocyte | |
| FAM3B | Protein FAM3B | P58499 |
| FAP | Prolyl endopeptidase FAP | Q12884 |
| FAS | Tumor necrosis factor receptor | P25445 |
| | superfamily member 6 | |
| FASLG | Tumor necrosis factor ligand | P48023 |
| | superfamily member 6 | |
| FBLN2 | Fibulin-2 | P98095 |
| FCRL2 | Fc receptor-like protein 2 | Q96LA5 |
| FGF5 | Fibroblast growth factor 5 | P12034 |
| FSHB | Follitropin subunit beta | P01225 |
| FSTL1 | Follistatin-related protein 1 | Q12841 |
| GAS6 | Growth arrest-specific protein 6 | Q14393 |
| GDF15 | Growth/differentiation factor 15 | Q99988 |
| GFAP | Glial fibrillary acidic protein | P14136 |
| GFRAL | GDNF family receptor alpha-like | Q6UXV0 |
| GHRL | Appetite-regulating hormone | Q9UBU3 |
| GIP | Gastric inhibitory polypeptide | P09681 |
| GIPC2 | PDZ domain-containing protein | Q8TF65 |
| | GIPC2 | |
| GP2 | Pancreatic secretory granule | P55259 |
| | membrane major glycoprotein | |
| | GP2 | |
| GZMB | Granzyme B | P10144 |
| HAVCR1 | Hepatitis A virus cellular receptor | Q96D42 |
| | 1 | |
| HMCN2 | Hemicentin-2 | Q8NDA2 |
| HSD11B1 | Corticosteroid 11-beta- | |
| | dehydrogenase isozyme 1 | |
| IGDCC4 | Immunoglobulin superfamily DCC | Q8TDY8 |
| | subclass member 4 | |
| IL17D | Interleukin-17D | Q8TAD2 |
| IL5RA | Interleukin-5 receptor subunit | Q01344 |
| | alpha | |
| IL7R | Interleukin-7 receptor subunit | P16871 |
| | alpha | |
| INSL3 | Insulin-like 3 | P51460 |
| ITGAV | Integrin alpha-V | P06756 |
| ITGB5 | Integrin beta-5 | P18084 |
| ITGBL1 | Integrin beta-like protein 1 | O95965 |
| KIF22 | Kinesin-like protein KIF22 | Q14807 |
| KIT | Mast/stem cell growth factor | P10721 |
| | receptor Kit | |
| KLK14 | Kallikrein-14 | Q9P0G3 |
| KLK3 | Prostate-specific antigen | P07288 |
| KLK4 | Kallikrein-4 | Q9Y5K2 |
| KLK7 | Kallikrein-7 | P49862 |
| KLK8 | Kallikrein-8 | O60259 |
| KLRF1 | Killer cell lectin-like receptor | Q9NZS2 |
| | subfamily F member 1 | |
| L1CAM | Neural cell adhesion molecule L1 | P32004 |
| LACRT | Extracellular glycoprotein lacritin | Q9GZZ8 |
| LECT2 | Leukocyte cell-derived | O14960 |
| | chemotaxin-2 | |
| LEG1 | Protein LEG1 homolog | Q6P5S2 |
| LHB | Lutropin subunit beta | P01229 |
| LMOD1 | Leiomodin-1 | P29536 |
| LPO | Lactoperoxidase | P22079 |
| LTBP2 | Latent-transforming growth factor | Q14767 |
| | beta-binding protein 2 | |
| LYPD3 | Ly6/PLAUR domain-containing | O95274 |
| | protein 3 | |
| MAMDC4 | Apical endosomal glycoprotein | Q6UXC1 |
| MATN3 | Matrilin-3 | O15232 |
| MEP1B | Meprin A subunit beta | Q16820 |
| MEPE | Matrix extracellular | Q9NQ76 |
| | phosphoglycoprotein | |
| MERTK | Tyrosine-protein kinase Mer | Q12866 |
| MFGE8 | Lactadherin | Q08431 |
| MLN | Promotilin | P12872 |
| MMP12 | Macrophage metalloelastase | P39900 |
| MOG | Myelin-oligodendrocyte | Q16653 |
| | glycoprotein | |
| MXRA8 | Matrix remodeling-associated | Q9BRK3 |
| | protein 8 | |
| NCAN | Neurocan core protein | O14594 |
| NEFL | Neurofilament light polypeptide | P07196 |
| NME3 | Nucleoside diphosphate kinase 3 | Q13232 |
| NOTCH3 | Neurogenic locus notch homolog | Q9UM47 |
| | protein 3 | |
| NPL | N-acetylneuraminate lyase | Q9BXD5 |
| NPTX2 | Neuronal pentraxin-2 | P47972 |
| NTF3 | Neurotrophin-3 | P20783 |
| NTF4 | Neurotrophin-4 | P34130 |
| NTproBNP | N-terminal prohormone of brain | NT-proBNP |
| | natriuretic peptide | |
| ODAM | Odontogenic ameloblast- | A1E959 |
| | associated protein | |
| PAEP | Glycodelin | P09466 |
| PAMR1 | Inactive serine protease PAMR1 | Q6UXH9 |
| PINLYP | phospholipase A2 inhibitor and | A6NC86 |
| | Ly6/PLAUR domain-containing | |
| | protein | |
| PKD1 | Polycystin-1 | P98161 |
| PLAT | Tissue-type plasminogen activator | P00750 |
| PODXL2 | Podocalyxin-like protein 2 | Q9NZ53 |
| POMC | Pro-opiomelanocortin | P01189 |
| PRELP | Prolargin | P51888 |
| PRL | Prolactin | P01236 |
| PRND | Prion-like protein doppel | Q9UKY0 |
| PROK1 | Prokineticin-1 | P58294 |
| PSPN | Persephin | O60542 |
| PTGDS | Prostaglandin-H2 D-isomerase | P41222 |
| PTN | Pleiotrophin | P21246 |
| PTPRM | Receptor-type tyrosine-protein | P28827 |
| | phosphatase mu | |
| PTPRN2 | Receptor-type tyrosine-protein | Q92932 |
| | phosphatase N2 | |
| PTPRR | Receptor-type tyrosine-protein | Q15256 |
| | phosphatase R | |
| PTPRZ1 | Receptor-type tyrosine-protein | P23471 |
| | phosphatase zeta | |
| REN | Renin | P00797 |
| RET | Proto-oncogene tyrosine-protein | P07949 |
| | kinase receptor Ret | |
| RGMA | Repulsive guidance molecule A | Q96B86 |
| RGMB | RGM domain family member B | |
| RLN2 | Prorelaxin H2 | P04090 |
| ROBO1 | Roundabout homolog 1 | Q9Y6N7 |
| RRM2 | Ribonucleoside-diphosphate | P31350 |
| | reductase subunit M2 | |
| SCARF2 | Scavenger receptor class F | Q96GP6 |
| | member 2 | |
| SCG2 | Secretogranin-2 | P13521 |
| SCG3 | Secretogranin-3 | Q8WXD2 |
| SCGB1A1 | Uteroglobin | P11684 |
| SDK2 | Protein sidekick-2 | Q58EX2 |
| SEPTIN3 | Neuronal-specific septin-3 | Q9UH03 |
| SOD2 | Superoxide dismutase [Mn], | P04179 |
| | mitochondrial | |
| SORCS2 | VPS10 domain-containing | Q96PQ0 |
| | receptor SorCS2 | |
| SOST | Sclerostin | Q9BQB4 |
| SPINK1 | Serine protease inhibitor Kazal- | P00995 |
| | type 1 | |
| SPON2 | Spondin-2 | Q9BUD6 |
| SPRR3 | Small proline-rich protein 3 | Q9UBC9 |
| SRPX | Sushi repeat-containing protein | P78539 |
| | SRPX | |
| SUSD2 | Sushi domain-containing protein 2 | Q9UGT4 |
| SUSD5 | Sushi domain-containing protein 5 | O60279 |
| TFF1 | Trefoil factor 1 | P04155 |
| THBS2 | Thrombospondin-2 | P35442 |
| TNFRSF11B | Tumor necrosis factor receptor | O00300 |
| | superfamily member 11B | |
| TNFRSF13B | Tumor necrosis factor receptor | O14836 |
| | superfamily member 13B | |
| TNFSF13 | Tumor necrosis factor ligand | O75888 |
| | superfamily member 13 | |
| TNXB | Tenascin-X | P22105 |
| TSPAN1 | Tetraspanin-1 | O60635 |
| WFDC2 | WAP four-disulfide core domain | Q14508 |
| | protein 2 | |
| WIF1 | Wnt inhibitory factor 1 | Q9Y5W5 |
| WNT9A | Protein Wnt-9a | O14904 |
| XCL1 | Lymphotactin | P47992 |

TABLE 6
Biomarkers significant in ProtAgeAccel20 model. A list of
all 20 biomarkers identified in the 20-biomarker aging model.
Further included are the UniProt ID for each protein.

| Gene name | Protein name | UniProt ID |
|---|---|---|
| ACRV1 | Acrosomal protein SP-10 | P26436 |
| AGRP | Agouti-related protein | O00253 |
| CDCP1 | CUB domain-containing protein 1 | Q9H5V8 |
| COL6A3 | Collagen alpha-3(VI) chain | P12111 |
| CXCL17 | C-X-C motif chemokine 17 | Q6UXB2 |
| EDA2R | Tumor necrosis factor receptor | Q9HAV5 |
| | superfamily member 27 | |
| ELN | Elastin | P15502 |
| ENG | Endoglin | P17813 |
| FSHB | Follitropin subunit beta | P01225 |
| GDF15 | Growth/differentiation factor 15 | Q99988 |
| GFAP | Glial fibrillary acidic protein | P14136 |
| IGDCC4 | Immunoglobulin superfamily DCC | Q8TDY8 |
| | subclass member 4 | |
| KLK3 | Prostate-specific antigen | P07288 |
| KLK7 | Kallikrein-7 | P49862 |
| LECT2 | Leukocyte cell-derived chemotaxin-2 | O14960 |
| LTBP2 | Latent-transforming growth factor | Q14767 |
| | beta-binding protein 2 | |
| NEFL | Neurofilament light polypeptide | P07196 |
| PODXL2 | Podocalyxin-like protein 2 | Q9NZ53 |
| PTPRR | Receptor-type tyrosine-protein | Q15256 |
| | phosphatase R | |
| SCARF2 | Scavenger receptor class F member 2 | Q96GP6 |

TABLE 7
Associations between ProtAgeAccel and biological aging phenotypes in
the full UK Biobank cohort (n = 45,441). Summary statistics from
linear regressions between ProtAgeAccel and all aging biomarkers tested.

| Outcome | Coefficient | Low_95%_CI | High_95%_CI | FDR P-value |
|---|---|---|---|---|
| Hand grip strength (right) | −0.0229 | −0.0257 | −0.0200 | 6.32E−55 |
| Hand grip strength (left) | −0.0221 | −0.0249 | −0.0193 | 6.31E−54 |
| Telomere length | −0.0186 | −0.0219 | −0.0152 | 9.30E−27 |
| IGF-1 | −0.0136 | −0.0169 | −0.0103 | 2.43E−15 |
| Lung function (FEV1) | −0.0135 | −0.0162 | −0.0107 | 2.42E−21 |
| Fluid intelligence | −0.0095 | −0.0127 | −0.0063 | 8.06E−09 |
| Albumin | −0.0087 | −0.0121 | −0.0054 | 5.02E−07 |
| Heel bone mineral density | −0.0073 | −0.0106 | −0.0041 | 1.15E−05 |
| Total bilirubin | −0.0023 | −0.0056 | 0.0010 | 1.87E−01 |
| ALT | 0.0007 | −0.0026 | 0.0041 | 6.65E−01 |
| BMI | 0.0079 | 0.0045 | 0.0113 | 4.64E−06 |
| GGT | 0.0083 | 0.0049 | 0.0117 | 1.81E−06 |
| Arterial stiffness index | 0.0095 | 0.0063 | 0.0127 | 8.06E−09 |
| AST | 0.0105 | 0.0071 | 0.0139 | 2.71E−09 |
| C-reactive protein | 0.0112 | 0.0078 | 0.0146 | 2.66E−10 |
| Reaction time | 0.0116 | 0.0083 | 0.0148 | 6.42E−12 |
| Systolic blood pressure | 0.0127 | 0.0093 | 0.0161 | 3.69E−13 |
| Diastolic blood pressure | 0.0128 | 0.0096 | 0.0160 | 8.51E−15 |
| Creatinine | 0.0158 | 0.0127 | 0.0188 | 7.24E−24 |
| Frequent insomnia | 0.0185 | 0.0107 | 0.0262 | 3.64E−06 |
| Frailty index (continuous) | 0.0258 | 0.0226 | 0.0291 | 1.89E−53 |
| Tired/lethargic every day | 0.0325 | 0.0189 | 0.0461 | 3.56E−06 |
| Sleep 10+ hours / day | 0.0404 | 0.0165 | 0.0644 | 1.02E−03 |
| Cystatin C | 0.0418 | 0.0387 | 0.0450 | 2.85E−145 |
| Self-rated facial aging | 0.0680 | 0.0482 | 0.0879 | 3.54E−11 |
| Slow walking pace | 0.0886 | 0.0762 | 0.1011 | 1.12E−43 |
| Poor self-rated health | 0.0981 | 0.0828 | 0.1135 | 1.94E−35 |

TABLE 8
Associations between ProtAgeAccel and functional and physiological
decline in the full UK Biobank cohort (n = 45,441). Summary
statistics from linear/logistic regressions between ProtAgeAccel
and all functional measures of physical and cognitive decline tested.

| Outcome | Coefficient | Low_95%_CI | High_95%_Cl | FDR P-value |
|---|---|---|---|---|
| Hand grip strength (right) | −0.0188 | −0.0230 | −0.0146 | 2.90E−17 |
| Hand grip strength (left) | −0.0158 | −0.0199 | −0.0117 | 5.22E−13 |
| Telomere length | −0.0158 | −0.0209 | −0.0108 | 3.32E−09 |
| IGF-1 | −0.0119 | −0.0167 | −0.0071 | 3.22E−06 |
| Lung function (FEV1) | −0.0069 | −0.0109 | −0.0029 | 1.15E−03 |
| Fluid intelligence | −0.0109 | −0.0158 | −0.0061 | 2.66E−05 |
| Albumin | −0.0019 | −0.0069 | 0.0030 | 4.74E−01 |
| Heel bone mineral density | −0.0079 | −0.0126 | −0.0031 | 2.11E−03 |
| Total bilirubin | −0.0039 | −0.0090 | 0.0012 | 1.53E−01 |
| ALT | 0.0052 | 0.0008 | 0.0095 | 2.70E−02 |
| BMI | 0.0066 | 0.0020 | 0.0111 | 6.46E−03 |
| GGT | 0.0047 | 0.0011 | 0.0084 | 1.55E−02 |
| Arterial stiffness index | 0.0087 | 0.0043 | 0.0130 | 2.03E−04 |
| AST | 0.0135 | 0.0095 | 0.0175 | 1.76E−10 |
| C-reactive protein | 0.0083 | 0.0041 | 0.0126 | 2.26E−04 |
| Reaction time | 0.0080 | 0.0035 | 0.0126 | 1.10E−03 |
| Systolic blood pressure | 0.0177 | 0.0127 | 0.0228 | 3.30E−11 |
| Diastolic blood pressure | 0.0156 | 0.0110 | 0.0203 | 1.90E−10 |
| Creatinine | 0.0074 | 0.0045 | 0.0104 | 3.17E−06 |
| Frequent insomnia | 0.0137 | 0.0013 | 0.0261 | 3.65E−02 |
| Frailty index (continuous) | 0.0064 | 0.0023 | 0.0105 | 3.41E−03 |
| Tired/lethargic every day | 0.0051 | −0.0186 | 0.0288 | 6.97E−01 |
| Sleep 10+ hours / day | 0.0084 | −0.0386 | 0.0554 | 7.25E−01 |
| Cystatin C | 0.0312 | 0.0280 | 0.0344 | 7.77E−80 |
| Self-rated facial aging | 0.0208 | −0.0124 | 0.0539 | 2.47E−01 |
| Slow walking pace | 0.0644 | 0.0377 | 0.0911 | 5.92E−06 |
| Poor self-rated health | 0.0507 | 0.0157 | 0.0857 | 6.46E−03 |

TABLE 9
Associations between ProtAgeAccel and biological aging phenotypes
in the subset of UK Biobank participants with no lifetime disease
diagnoses (n = 20,353). Summary statistics from linear regressions
between ProtAgeAccel and all aging biomarkers tested.

| Outcome | Coefficient | Low_95%_CI | High_95%_CI | FDR P-value |
|---|---|---|---|---|
| Hand grip strength (right) | −0.0188 | −0.0211 | −0.0165 | 1.76E−56 |
| Hand grip strength (left) | −0.0178 | −0.0200 | −0.0155 | 4.92E−53 |
| Telomere length | −0.0206 | −0.0233 | −0.0179 | 3.91E−49 |
| IGF-1 | −0.0129 | −0.0156 | −0.0103 | 7.11E−21 |
| Lung function (FEV1) | −0.0124 | −0.0146 | −0.0101 | 4.15E−27 |
| Fluid intelligence | −0.0072 | −0.0098 | −0.0046 | 5.58E−08 |
| Albumin | −0.0197 | −0.0224 | −0.0170 | 4.72E−45 |
| Heel bone mineral density | −0.0077 | −0.0104 | −0.0051 | 1.35E−08 |
| Total bilirubin | −0.0061 | −0.0088 | −0.0034 | 1.06E−05 |
| ALT | 0.0170 | 0.0143 | 0.0197 | 2.96E−34 |
| BMI | 0.0036 | 0.0009 | 0.0064 | 9.58E−03 |
| GGT | 0.0169 | 0.0141 | 0.0196 | 2.36E−33 |
| Arterial stiffness index | 0.0071 | 0.0045 | 0.0096 | 1.13E−07 |
| AST | 0.0274 | 0.0246 | 0.0301 | 4.67E−83 |
| C-reactive protein | 0.0213 | 0.0186 | 0.0241 | 8.56E−51 |
| Reaction time | 0.0094 | 0.0068 | 0.0121 | 3.48E−12 |
| Systolic blood pressure | 0.0035 | 0.0008 | 0.0063 | 1.23E−02 |
| Diastolic blood pressure | −0.0003 | −0.0029 | 0.0023 | 8.26E−01 |
| Creatinine | 0.0186 | 0.0162 | 0.0211 | 3.71E−49 |
| Frequent insomnia | 0.0269 | 0.0206 | 0.0332 | 1.17E−16 |
| Frailty index (continuous) | 0.0258 | 0.0232 | 0.0284 | 3.49E−80 |
| Tired/lethargic every day | 0.0476 | 0.0365 | 0.0586 | 4.86E−17 |
| Sleep 10+ hours / day | 0.0376 | 0.0179 | 0.0573 | 2.11E−04 |
| Cystatin C | 0.0448 | 0.0422 | 0.0474 | 1.48E−253 |
| Self-rated facial aging | 0.0613 | 0.0452 | 0.0774 | 1.32E−13 |
| Slow walking pace | 0.0886 | 0.0783 | 0.0990 | 5.81E−63 |
| Poor self-rated health | 0.1122 | 0.0996 | 0.1249 | 6.55E−67 |

TABLE 10
Associations between ProtAgeAccel and functional and physiological decline
in the subset of UK Biobank participants with no lifetime disease diagnoses
(n = 20,353). Summary statistics from linear/logistic regressions between
ProtAgeAccel and all functional measures of physical and cognitive decline tested.

| Outcome | Coefficient | Low_95%_CI | High_95%_CI | FDR P-value |
|---|---|---|---|---|
| Hand grip strength (right) | −0.0139 | −0.0173 | −0.0105 | 3.97E−15 |
| Hand grip strength (left) | −0.0115 | −0.0148 | −0.0082 | 3.84E−11 |
| Telomere length | −0.0187 | −0.0228 | −0.0147 | 1.16E−18 |
| IGF-1 | −0.0107 | −0.0146 | −0.0069 | 1.46E−07 |
| Lung function (FEV1) | −0.0061 | −0.0093 | −0.0029 | 3.51E−04 |
| Fluid intelligence | −0.0066 | −0.0105 | −0.0027 | 1.38E−03 |
| Albumin | −0.0102 | −0.0142 | −0.0063 | 1.04E−06 |
| Heel bone mineral density | −0.0069 | −0.0108 | −0.0031 | 6.31E−04 |
| Total bilirubin | −0.0077 | −0.0118 | −0.0036 | 3.65E−04 |
| ALT | 0.0178 | 0.0143 | 0.0213 | 3.85E−22 |
| BMI | 0.0007 | −0.0029 | 0.0044 | 7.23E−01 |
| GGT | 0.0079 | 0.0049 | 0.0108 | 4.70E−07 |
| Arterial stiffness index | 0.0060 | 0.0025 | 0.0094 | 1.18E−03 |
| AST | 0.0256 | 0.0224 | 0.0289 | 4.04E−54 |
| C-reactive protein | 0.0154 | 0.0120 | 0.0188 | 2.62E−18 |
| Reaction time | 0.0047 | 0.0011 | 0.0084 | 1.34E−02 |
| Systolic blood pressure | 0.0054 | 0.0013 | 0.0094 | 1.22E−02 |
| Diastolic blood pressure | 0.0022 | −0.0016 | 0.0059 | 2.78E−01 |
| Creatinine | 0.0111 | 0.0087 | 0.0134 | 9.81E−19 |
| Frequent insomnia | 0.0211 | 0.0112 | 0.0311 | 6.27E−05 |
| Frailty index (continuous) | 0.0077 | 0.0044 | 0.0110 | 1.09E−05 |
| Tired/lethargic every day | 0.0222 | 0.0031 | 0.0412 | 2.51E−02 |
| Sleep 10+ hours / day | 0.0005 | −0.0377 | 0.0387 | 9.79E−01 |
| Cystatin C | 0.0329 | 0.0304 | 0.0355 | 2.13E−137 |
| Self-rated facial aging | 0.0344 | 0.0078 | 0.0610 | 1.34E−02 |
| Slow walking pace | 0.0619 | 0.0403 | 0.0834 | 5.89E−08 |
| Poor self-rated health | 0.0547 | 0.0266 | 0.0828 | 2.50E−04 |

TABLE 11
Associations between ProtAgeAccel and mortality and incident non-
cancer diseases (Model 1) in the full UK Biobank population (n =
45,441). Summary statistics from Cox proportional hazards models
between ProtAgeAccel and all-cause mortality and incidence of all
non-cancer illnesses using model 1 covariates (age and sex).

| | Hazard | Low | High | FDR |
|---|---|---|---|---|
| Outcome | Ratio | 95% CI | 95% CI | P-value |
| Type II diabetes | 1.0349 | 1.0202 | 1.0497 | 3.46E−06 |
| Parkinson's disease | 1.0369 | 0.9988 | 1.0764 | 5.78E−02 |
| Rheumatoid arthritis | 1.0465 | 1.0206 | 1.0732 | 4.16E−04 |
| Chronic liver diseases | 1.0471 | 1.0232 | 1.0715 | 1.06E−04 |
| Osteoarthritis | 1.0477 | 1.0375 | 1.0581 | 5.05E−20 |
| Macular degeneration | 1.0501 | 1.0250 | 1.0759 | 9.24E−05 |
| Ischemic heart disease | 1.0570 | 1.0453 | 1.0688 | 7.42E−22 |
| Osteoporosis | 1.0772 | 1.0571 | 1.0978 | 2.35E−14 |
| All stroke | 1.0781 | 1.0558 | 1.1008 | 2.78E−12 |
| Ischemic stroke | 1.0813 | 1.0573 | 1.1059 | 1.34E−11 |
| Emphysema, COPD | 1.0886 | 1.0703 | 1.1071 | 3.30E−22 |
| All-cause mortality | 1.1068 | 1.0944 | 1.1194 | 1.19E−68 |
| Chronic kidney | 1.1080 | 1.0912 | 1.1251 | 1.40E−38 |
| diseases | | | | |
| All-cause dementia | 1.1298 | 1.1016 | 1.1587 | 8.43E−21 |
| Alzheimer's disease | 1.1559 | 1.1173 | 1.1957 | 1.17E−16 |

TABLE 12
Associations between ProtAgeAccel and mortality and incident
non-cancer diseases (Model 2) in the full UK Biobank population
(n = 45,441). Summary statistics from Cox proportional
hazards models between ProtAgeAccel and all-cause mortality
and incidence of all non-cancer illnesses using model 2 covariates
(age, sex, ethnicity, Townsend deprivation index, recruitment
centre, IPAQ activity group, and smoking status).

| | Hazard | Low | High | FDR |
|---|---|---|---|---|
| Outcome | Ratio | 95% CI | 95% CI | P-value |
| Parkinson's disease | 1.0321 | 0.9940 | 1.0716 | 9.98E−02 |
| Chronic liver diseases | 1.0383 | 1.0147 | 1.0624 | 1.46E−03 |
| Type II diabetes | 1.0412 | 1.0265 | 1.0560 | 3.26E−08 |
| Rheumatoid arthritis | 1.0446 | 1.0187 | 1.0711 | 7.55E−04 |
| Osteoarthritis | 1.0461 | 1.0358 | 1.0565 | 1.45E−18 |
| Macular degeneration | 1.0513 | 1.0261 | 1.0772 | 6.75E−05 |
| Ischemic heart disease | 1.0557 | 1.0440 | 1.0676 | 6.21E−21 |
| Osteoporosis | 1.0752 | 1.0549 | 1.0959 | 1.71E−13 |
| All stroke | 1.0817 | 1.0593 | 1.1046 | 3.14E−13 |
| Ischemic stroke | 1.0849 | 1.0607 | 1.1097 | 2.08E−12 |
| Emphysema, COPD | 1.0871 | 1.0689 | 1.1057 | 1.37E−21 |
| All-cause mortality | 1.1061 | 1.0937 | 1.1188 | 6.03E−67 |
| Chronic kidney | 1.1118 | 1.0949 | 1.1289 | 3.08E−41 |
| diseases | | | | |
| All-cause dementia | 1.1339 | 1.1055 | 1.1632 | 1.37E−21 |
| Alzheimer's disease | 1.1610 | 1.1219 | 1.2015 | 2.94E−17 |

TABLE 13
Associations between ProtAgeAccel and mortality and incident non-
cancer diseases (Model 3) in the full UK Biobank population (n =
45,441). Summary statistics from Cox proportional hazards models
between ProtAgeAccel and all-cause mortality and incidence
of all non-cancer illnesses using model 2 covariates (age, sex,
ethnicity, Townsend deprivation index, recruitment centre, IPAQ
activity group, smoking status, BMI, and prevalent hypertension).

| | Hazard | Low | High | FDR |
|---|---|---|---|---|
| Outcome | Ratio | 95% CI | 95% CI | P-value |
| Chronic liver diseases | 1.0256 | 1.0025 | 1.0493 | 3.20E−02 |
| Type II diabetes | 1.0268 | 1.0125 | 1.0413 | 2.63E−04 |
| Parkinson's disease | 1.0319 | 0.9937 | 1.0715 | 1.03E−01 |
| Rheumatoid arthritis | 1.0392 | 1.0135 | 1.0655 | 2.98E−03 |
| Osteoarthritis | 1.0434 | 1.0331 | 1.0538 | 1.06E−16 |
| Macular degeneration | 1.0479 | 1.0228 | 1.0737 | 2.17E−04 |
| Ischemic heart disease | 1.0494 | 1.0378 | 1.0612 | 6.68E−17 |
| All stroke | 1.0733 | 1.0511 | 1.0960 | 5.58E−11 |
| Osteoporosis | 1.0746 | 1.0543 | 1.0954 | 3.15E−13 |
| Ischemic stroke | 1.0755 | 1.0516 | 1.1000 | 3.48E−10 |
| Emphysema, COPD | 1.0810 | 1.0628 | 1.0994 | 7.87E−19 |
| All-cause mortality | 1.1008 | 1.0884 | 1.1133 | 1.11E−60 |
| Chronic kidney | 1.1010 | 1.0844 | 1.1179 | 1.72E−34 |
| diseases | | | | |
| All-cause dementia | 1.1292 | 1.1007 | 1.1583 | 4.98E−20 |
| Alzheimer's disease | 1.1570 | 1.1180 | 1.1975 | 1.85E−16 |

TABLE 14
Associations between ProtAgeAccel20 and mortality and incident non-
cancer diseases (Model 1) in the full UK Biobank population (n =
45,441). Summary statistics from Cox proportional hazards models
between ProtAgeAccel and all-cause mortality and incidence of all
non-cancer illnesses using model 1 covariates (age and sex).

| | Hazard | Low | High | FDR |
|---|---|---|---|---|
| Outcome | Ratio | 95% CI | 95% CI | P-value |
| Type II diabetes | 1.0341 | 1.0222 | 1.0462 | 1.82E−08 |
| Parkinson's disease | 1.0351 | 1.0032 | 1.0680 | 3.10E−02 |
| Rheumatoid arthritis | 1.0456 | 1.0243 | 1.0673 | 2.25E−05 |
| Chronic liver diseases | 1.0877 | 1.0677 | 1.1082 | 1.65E−18 |
| Osteoarthritis | 1.0373 | 1.0290 | 1.0456 | 1.00E−18 |
| Macular degeneration | 1.0462 | 1.0249 | 1.0679 | 1.87E−05 |
| Ischemic heart disease | 1.0492 | 1.0397 | 1.0588 | 2.03E−24 |
| Osteoporosis | 1.0772 | 1.0603 | 1.0943 | 6.08E−20 |
| All stroke | 1.0580 | 1.0398 | 1.0765 | 2.56E−10 |
| Ischemic stroke | 1.0617 | 1.0420 | 1.0817 | 4.73E−10 |
| Emphysema, COPD | 1.0994 | 1.0839 | 1.1150 | 9.95E−39 |
| All-cause mortality | 1.1125 | 1.1019 | 1.1232 | 9.45E−105 |
| Chronic kidney | 1.1145 | 1.1001 | 1.1291 | 4.14E−59 |
| diseases | | | | |
| All-cause dementia | 1.1203 | 1.0955 | 1.1458 | 9.72E−23 |
| Alzheimer's disease | 1.1344 | 1.1003 | 1.1695 | 8.79E−16 |

TABLE 15
Associations between ProtAgeAccel20 and mortality and incident
non-cancer diseases (Model 2) in the full UK Biobank population
(n = 45,441). Summary statistics from Cox proportional
hazards models between ProtAgeAccel20 and all-cause mortality
and incidence of all non-cancer illnesses using model 2 covariates
(age, sex, ethnicity, Townsend deprivation index, recruitment
centre, IPAQ activity group, and smoking status).

| | Hazard | Low | High | FDR |
|---|---|---|---|---|
| Outcome | Ratio | 95% CI | 95% CI | P-value |
| Parkinson's disease | 1.0327 | 1.0007 | 1.0658 | 4.51E−02 |
| Chronic liver diseases | 1.0767 | 1.0568 | 1.0969 | 1.27E−14 |
| Type II diabetes | 1.0381 | 1.0261 | 1.0502 | 4.78E−10 |
| Rheumatoid arthritis | 1.0434 | 1.0221 | 1.0652 | 5.73E−05 |
| Osteoarthritis | 1.0348 | 1.0265 | 1.0433 | 2.71E−16 |
| Macular degeneration | 1.0466 | 1.0251 | 1.0684 | 1.92E−05 |
| Ischemic heart disease | 1.0446 | 1.0350 | 1.0542 | 4.19E−20 |
| Osteoporosis | 1.0747 | 1.0577 | 1.0920 | 2.21E−18 |
| All stroke | 1.0565 | 1.0383 | 1.0751 | 8.38E−10 |
| Ischemic stroke | 1.0594 | 1.0397 | 1.0796 | 2.26E−09 |
| Emphysema, COPD | 1.0833 | 1.0680 | 1.0989 | 2.06E−27 |
| All-cause mortality | 1.1061 | 1.0955 | 1.1168 | 7.23E−92 |
| Chronic kidney | 1.1164 | 1.1018 | 1.1311 | 6.51E−60 |
| diseases | | | | |
| All-cause dementia | 1.1214 | 1.0963 | 1.1471 | 1.44E−22 |
| Alzheimer's disease | 1.1361 | 1.1016 | 1.1718 | 9.77E−16 |

TABLE 16
Associations between ProtAgeAccel20 and mortality and incident non-
cancer diseases (Model 3) in the full UK Biobank population (n =
45,441). Summary statistics from Cox proportional hazards models
between ProtAgeAccel20 and all-cause mortality and incidence of
all non-cancer illnesses using model 2 covariates (age, sex, ethnicity,
Townsend deprivation index, recruitment centre, IPAQ activity group,
smoking status, BMI, and prevalent hypertension).

| | Hazard | Low | High | FDR |
|---|---|---|---|---|
| Outcome | Ratio | 95% CI | 95% CI | P-value |
| Chronic liver diseases | 1.0678 | 1.0482 | 1.0879 | 7.66E−12 |
| Type II diabetes | 1.0283 | 1.0165 | 1.0403 | 2.84E−06 |
| Parkinson's disease | 1.0327 | 1.0006 | 1.0658 | 4.60E−02 |
| Rheumatoid arthritis | 1.0409 | 1.0197 | 1.0625 | 1.47E−04 |
| Osteoarthritis | 1.0337 | 1.0254 | 1.0422 | 2.09E−15 |
| Macular degeneration | 1.0449 | 1.0235 | 1.0668 | 3.68E−05 |
| Ischemic heart disease | 1.0411 | 1.0316 | 1.0507 | 2.24E−17 |
| All stroke | 1.0516 | 1.0335 | 1.0700 | 2.08E−08 |
| Osteoporosis | 1.0724 | 1.0555 | 1.0897 | 2.24E−17 |
| Ischemic stroke | 1.0539 | 1.0343 | 1.0739 | 5.62E−08 |
| Emphysema, COPD | 1.0795 | 1.0642 | 1.0950 | 3.88E−25 |
| All-cause mortality | 1.1027 | 1.0921 | 1.1134 | 2.06E−86 |
| Chronic kidney | 1.1106 | 1.0962 | 1.1253 | 9.86E−55 |
| diseases | | | | |
| All-cause dementia | 1.1183 | 1.0932 | 1.1439 | 1.53E−21 |
| Alzheimer's disease | 1.1334 | 1.0989 | 1.1689 | 3.36E−15 |

TABLE 17
Associations between ProtAgeAccel and mortality and incident
cancers (Model 1) in the full UK Biobank population (n =
45,441). Summary statistics from Cox proportional hazards
models between ProtAgeAccel and all-cause mortality and incidence
of cancers using model 1 covariates (age and sex).

| | Hazard | Low | High | FDR |
|---|---|---|---|---|
| Outcome | Ratio | 95% CI | 95% CI | P-value |
| Hodgkin lymphoma | 0.9666 | 0.8338 | 1.1206 | 7.12E−01 |
| Breast cancer | 0.9897 | 0.9648 | 1.0152 | 5.08E−01 |
| Ovarian cancer | 0.9955 | 0.9320 | 1.0634 | 8.94E−01 |
| Colorectal cancer | 1.0184 | 0.9875 | 1.0501 | 3.69E−01 |
| Leukemia | 1.0307 | 0.9690 | 1.0964 | 4.49E−01 |
| Pancreatic cancer | 1.0379 | 0.9761 | 1.1035 | 3.69E−01 |
| Prostate cancer | 1.0465 | 1.0230 | 1.0705 | 1.03E−03 |
| Brain cancer | 1.0523 | 0.9740 | 1.1369 | 3.69E−01 |
| Liver cancer | 1.0554 | 0.9730 | 1.1449 | 3.69E−01 |
| Lung cancer | 1.0638 | 1.0282 | 1.1007 | 2.22E−03 |
| Esophageal cancer | 1.0800 | 1.0151 | 1.1490 | 4.47E−02 |
| Non-Hodgkin lymphoma | 1.0824 | 1.0294 | 1.1382 | 7.97E−03 |

TABLE 18
Associations between ProtAgeAccel and mortality and incident cancers
(Model 2) in the full UK Biobank population (n = 45,441).
Summary statistics from Cox proportional hazards models between
ProtAgeAccel and all-cause mortality and incidence of cancers using
model 2 covariates (age, sex, ethnicity, Townsend deprivation index,
recruitment centre, IPAQ activity group, and smoking status).

| | Hazard | Low | High | FDR |
|---|---|---|---|---|
| Outcome | Ratio | 95% CI | 95% CI | P-value |
| Hodgkin lymphoma | 0.9703 | 0.8370 | 1.1248 | 7.52E−01 |
| Breast cancer | 0.9885 | 0.9636 | 1.0140 | 4.62E−01 |
| Ovarian cancer | 0.9903 | 0.9272 | 1.0576 | 7.71E−01 |
| Colorectal cancer | 1.0157 | 0.9849 | 1.0474 | 4.62E−01 |
| Leukemia | 1.0277 | 0.9662 | 1.0931 | 4.62E−01 |
| Pancreatic cancer | 1.0349 | 0.9736 | 1.1001 | 4.62E−01 |
| Prostate cancer | 1.0475 | 1.0239 | 1.0715 | 3.80E−04 |
| Liver cancer | 1.0492 | 0.9677 | 1.1376 | 4.62E−01 |
| Brain cancer | 1.0528 | 0.9742 | 1.1377 | 4.62E−01 |
| Lung cancer | 1.0725 | 1.0365 | 1.1097 | 3.80E−04 |
| Esophageal cancer | 1.0794 | 1.0142 | 1.1488 | 4.88E−02 |
| Non-Hodgkin lymphoma | 1.0794 | 1.0267 | 1.1349 | 1.12E−02 |

TABLE 19
Associations between ProtAgeAccel and mortality and incident
cancers (Model 3) in the full UK Biobank population (n =
45,441). Summary statistics from Cox proportional hazards models
between ProtAgeAccel and all-cause mortality and incidence
of cancers using model 2 covariates (age, sex, ethnicity, Townsend
deprivation index, recruitment centre, IPAQ activity group,
smoking status, BMI, and prevalent hypertension).

| | Hazard | Low | High | FDR |
|---|---|---|---|---|
| Outcome | Ratio | 95% CI | 95% CI | P-value |
| Hodgkin lymphoma | 0.9693 | 0.8359 | 1.1241 | 7.02E−01 |
| Ovarian cancer | 0.9872 | 0.9243 | 1.0545 | 7.02E−01 |
| Breast cancer | 0.9886 | 0.9637 | 1.0141 | 4.54E−01 |
| Colorectal cancer | 1.0169 | 0.9860 | 1.0488 | 4.54E−01 |
| Leukemia | 1.0299 | 0.9681 | 1.0957 | 4.54E−01 |
| Pancreatic cancer | 1.0354 | 0.9740 | 1.1006 | 4.54E−01 |
| Liver cancer | 1.0432 | 0.9623 | 1.1309 | 4.54E−01 |
| Prostate cancer | 1.0488 | 1.0251 | 1.0731 | 5.17E−04 |
| Brain cancer | 1.0555 | 0.9765 | 1.1409 | 4.16E−01 |
| Lung cancer | 1.0698 | 1.0339 | 1.1071 | 6.61E−04 |
| Esophageal cancer | 1.0752 | 1.0102 | 1.1444 | 6.83E−02 |
| Non-Hodgkin lymphoma | 1.0790 | 1.0261 | 1.1345 | 1.20E−02 |

TABLE 20
Age-specific incidence rates in the UK Biobank for mortality
and age-related diseases by ProtAgeAccel (PAA) deciles.
Cumulative incidence rates are shown for those who are
aged 50, 55, 60, and 65 years at recruitment in the UK
Biobank (n = 45,441). Incidence rates are for the
11-16 years after recruitment in the UK Biobank.

| | ProtAgeAccel | 50 | 55 | 60 | 65 |
|---|---|---|---|---|---|
| Outcome | decile | years | years | years | years |
| All-cause mortality | Top 10% | 2.78 | 7.34 | 19.07 | 60.02 |
| | Median 10% | 0.43 | 1.11 | 2.87 | 12.60 |
| | Bottom 10% | 0.05 | 0.24 | 0.62 | 3.99 |
| Type II diabetes | Top 10% | 2.67 | 6.33 | 13.47 | 47.49 |
| | Median 10% | 0.62 | 1.30 | 3.53 | 8.99 |
| | Bottom 10% | 0.10 | 0.30 | 1.14 | 3.75 |
| Ischemic heart disease | Top 10% | 3.26 | 8.76 | 22.04 | 47.60 |
| | Median 10% | 1.12 | 2.28 | 5.02 | 14.65 |
| | Bottom 10% | 0.16 | 0.67 | 1.58 | 5.34 |
| All stroke | Top 10% | 1.27 | 2.57 | 6.24 | 10.53 |
| | Median 10% | 0.24 | 0.36 | 0.81 | 4.60 |
| | Bottom 10% | 0.00 | 0.10 | 0.37 | 1.38 |
| Ischemic stroke | Top 10% | 1.09 | 2.12 | 6.12 | 9.50 |
| | Median 10% | 0.19 | 0.26 | 0.55 | 3.57 |
| | Bottom 10% | 0.00 | 0.10 | 0.26 | 0.96 |
| Emphysema, COPD | Top 10% | 2.02 | 4.87 | 11.91 | 28.23 |
| | Median 10% | 0.24 | 0.99 | 1.92 | 6.08 |
| | Bottom 10% | 0.00 | 0.05 | 0.50 | 2.15 |
| Chronic liver diseases | Top 10% | 1.29 | 2.97 | 6.23 | 10.96 |
| | Median 10% | 0.20 | 0.48 | 1.23 | 3.12 |
| | Bottom 10% | 0.00 | 0.05 | 0.10 | 1.02 |
| Chronic kidney | Top 10% | 1.91 | 6.27 | 15.36 | 53.27 |
| diseases | Median 10% | 0.28 | 0.63 | 2.09 | 9.21 |
| | Bottom 10% | 0.00 | 0.15 | 0.32 | 2.10 |
| All-cause dementia | Top 10% | 0.37 | 0.99 | 4.04 | 30.57 |
| | Median 10% | 0.05 | 0.05 | 0.36 | 2.84 |
| | Bottom 10% | 0.00 | 0.00 | 0.05 | 0.41 |
| Alzheimer's disease | Top 10% | 0.13 | 0.90 | 1.70 | 12.49 |
| | Median 10% | 0.05 | 0.11 | 0.26 | 1.32 |
| | Bottom 10% | 0.00 | 0.05 | 0.05 | 0.35 |
| Parkinson's disease | Top 10% | 0.07 | 0.18 | 1.68 | 5.70 |
| | Median 10% | 0.00 | 0.06 | 0.28 | 1.32 |
| | Bottom 10% | 0.00 | 0.00 | 0.05 | 0.22 |
| Rheumatoid arthritis | Top 10% | 0.94 | 2.17 | 5.33 | 26.06 |
| | Median 10% | 0.41 | 0.71 | 1.14 | 4.09 |
| | Bottom 10% | 0.05 | 0.30 | 0.68 | 1.47 |
| Macular degeneration | Top 10% | 0.12 | 0.82 | 4.14 | 14.09 |
| | Median 10% | 0.05 | 0.51 | 1.63 | 5.69 |
| | Bottom 10% | 0.00 | 0.10 | 0.26 | 1.35 |
| Osteoporosis | Top 10% | 1.58 | 4.58 | 14.48 | 44.63 |
| | Median 10% | 0.48 | 1.03 | 2.50 | 8.93 |
| | Bottom 10% | 0.20 | 0.35 | 0.80 | 4.04 |
| Osteoarthritis | Top 10% | 7.58 | 18.69 | 40.15 | 76.65 |
| | Median 10% | 2.21 | 4.92 | 11.53 | 27.47 |
| | Bottom 10% | 0.41 | 1.49 | 3.51 | 10.63 |

TABLE 21
Age-specific incidence rates in the China Kadoorie Biobank for mortality and
age-related diseases by ProtAgeAccel (PAA) deciles. Cumulative incidence
rates are shown for those who are aged 35, 40, 45, 50, 55, 60, and 65 years
at recruitment in the China Kadoorie Biobank (n = 2,026). Incidence
rates are for the 11-14 years after recruitment in the China Kadoorie Biobank.

| | ProtAgeAccel | 35 | 40 | 45 | 50 | 55 | 60 | 65 |
|---|---|---|---|---|---|---|---|---|
| Outcome | decile | years | years | years | years | years | years | years |
| All-cause mortality | Top 10% | 0.53 | 2.64 | 4.65 | 7.63 | 19.82 | 32.65 | 32.65 |
| | Median 10% | 0.00 | 0.00 | 0.57 | 0.57 | 3.39 | 7.57 | 7.57 |
| | Bottom 10% | 0.00 | 0.00 | 0.00 | 0.00 | 1.24 | 1.93 | 4.94 |
| All stroke | Top 10% | 0.00 | 1.97 | 3.17 | 12.09 | 22.09 | 34.55 | 47.64 |
| | Median 10% | 0.00 | 0.52 | 1.85 | 2.78 | 5.42 | 10.65 | 18.74 |
| | Bottom 10% | 0.00 | 0.00 | 0.00 | 1.06 | 2.18 | 4.29 | 11.00 |
| Ischemic stroke | Top 10% | 0.00 | 1.97 | 3.17 | 8.67 | 19.06 | 32.01 | 45.61 |
| | Median 10% | 0.00 | 0.52 | 1.85 | 2.78 | 5.42 | 7.67 | 16.03 |
| | Bottom 10% | 0.00 | 0.00 | 0.00 | 1.06 | 2.18 | 4.29 | 8.94 |
| Ischemic heart | Top 10% | 0.00 | 1.89 | 5.09 | 6.41 | 20.77 | 28.69 | 28.69 |
| disease | Median 10% | 0.00 | 0.00 | 0.70 | 3.96 | 6.95 | 8.70 | 27.56 |
| | Bottom 10% | 0.00 | 0.00 | 0.00 | 0.54 | 1.13 | 2.66 | 11.68 |
| Type II diabetes | Top 10% | 0.00 | 0.00 | 0.00 | 0.00 | 6.52 | 6.52 | 6.52 |
| | Median 10% | 0.00 | 0.00 | 1.47 | 3.93 | 6.34 | 10.47 | 14.74 |
| | Bottom 10% | 0.00 | 0.00 | 0.00 | 0.00 | 1.96 | 3.55 | 4.80 |
| Emphysema, COPD | Top 10% | 0.00 | 0.86 | 0.86 | 0.86 | 13.49 | 13.49 | 35.12 |
| | Median 10% | 0.00 | 0.00 | 0.00 | 2.45 | 2.45 | 4.48 | 4.48 |
| | Bottom 10% | 0.00 | 0.00 | 0.00 | 0.00 | 0.00 | 1.75 | 4.13 |
| Chronic liver | Top 10% | 0.00 | 0.00 | 0.00 | 0.00 | 4.04 | 4.04 | 4.04 |
| diseases | Median 10% | 0.00 | 0.00 | 0.00 | 0.00 | 0.00 | 0.00 | 0.00 |
| | Bottom 10% | 0.00 | 0.00 | 0.00 | 0.00 | 0.59 | 0.59 | 0.59 |
| Chronic kidney | Top 10% | 0.00 | 1.41 | 3.86 | 5.78 | 8.40 | 14.94 | 14.94 |
| diseases | Median 10% | 0.00 | 0.00 | 0.00 | 0.00 | 0.00 | 0.00 | 0.00 |
| | Bottom 10% | 0.00 | 0.00 | 0.00 | 0.00 | 0.00 | 0.00 | 0.00 |

TABLE 22
Individual aging biomarker and frailty variables tested in
the UK Biobank. Descriptions and Field IDs for variables
used in aging biomarker and functional outcome analyses.

| | Field ID |
|---|---|
| Biomarkers | |
| Alanine aminotransferase | 30620 |
| Albumin | 30600 |
| Aspartate aminotransferase | 30650 |
| High sensitivity C-reactive protein | 30710 |
| Creatinine | 30700 |
| Cystatin C | 30720 |
| Total bilirubin | 30840 |
| Gamma glutamyltransferase | 30730 |
| Insulin-like growth factor 1 (IGF-1) | 30770 |
| Leukocyte telomere length | 22192 |
| Physical measures | |
| Usual walking pace | 924 |
| Body mass index (BMI) | 21001 |
| Self-rated health | 2178 |
| Facial aging | 1757 |
| Hours of sleep | 1160 |
| Tiredness | 2080 |
| Insomnia | 1200 |
| Systolic blood pressure | 4080 |
| Diastolic blood pressure | 4079 |
| Arterial stiffness index | 21021 |
| Heel bone mineral density | 3148 |
| Lung function (FEV1) best measure | 20150 |
| Hand grip strength (left) | 46 |
| Hand grip strength (right) | 47 |
| Cognitive measures | |
| Reaction time | 20023 |
| Fluid intelligence score | 20016 |

TABLE 23
Items used to construct the frailty index in the UK Biobank. Descriptions
and Field IDs for variables used to construct the summary frailty index.

| Type of | | | | | |
|---|---|---|---|---|---|
| deficit | Item | Trait | Field ID | Categories | Coding in Frailty Index |
| Sensory | 1 | Glaucoma * | 20002 | no, yes | Categorized 0/1 |
| | 2 | Cataracts * | 20002 | no, yes | Categorized 0/1 |
| | 3 | Hearing | 2247 | no, yes, | Categorized 0/1 |
| | | difficulty | | completely deaf | (combined yes/deaf |
| | | | | | groups as 1) |
| Cranial | 4 | Migraine * | 20002 | no, yes | Categorized 0/1 |
| | 5 | Dental | 6149 | ulcers, painful | Categorized 0/1 for none |
| | | problems | | gums, bleeding | vs. any |
| | | | | gums, loose | |
| | | | | teeth, | |
| | | | | toothache, | |
| | | | | dentures | |
| Mental | 6 | Self-rated | 2178 | excellent, good, | 0—excellent; |
| wellbeing | | health | | fair, poor | 0.25—good; |
| | | | | | 0.5—fair, |
| | | | | | 1—poor |
| | 7 | Fatigue: | 2080 | not at all, | 0, 0.25, 0.5, 1, |
| | | frequency of | | several days, | respectively |
| | | tiredness/ | | more than half, | |
| | | lethargy in | | nearly every | |
| | | last two weeks | | day | |
| | 8 | Sleep: | 1200 | never/rarely, | Categorized 0, 0.5, 1, |
| | | experience of | | sometimes, | respectively |
| | | sleeplessness/ | | usually | |
| | | insomnia | | | |
| | 9 | Depressed | 2050 | not at all, | 0—not at all, |
| | | feelings: | | several days, | 0.5—several days, |
| | | frequency in | | more than half, | 0.75—more than half, |
| | | last two weeks | | nearly every | 1—nearly every day |
| | | | | day | |
| | 10 | Self-described | 1970 | no, yes | Categorized 0/1 |
| | | nervous | | | |
| | | personality | | | |
| | 11 | Severe anxiety/ | 20002 | no, yes | Categorized 0/1 |
| | | panic attacks * | | | |
| | 12 | Common to feel | 2020 | no, yes | Categorized 0/1 |
| | | loneliness | | | |
| | 13 | Sense of misery | 1930 | no, yes | Categorized 0/1 |
| | | (ever/never) | | | |
| Infirmity | 14 | Infirmity: | 2188 | no, yes | Categorized 0/1 |
| | | long-standing | | | |
| | | illness or | | | |
| | | disability | | | |
| | 15 | Falls in last | 2296 | categorical: no | 0, 0.5, 1, respectively |
| | | year | | falls, one fall, | |
| | | | | more than one | |
| | 16 | Fractures/ | 2463 | no, yes | Categorized 0/1 |
| | | broken bones | | | |
| | | in last five | | | |
| | | years | | | |
| Cardiometabolic | 17 | Diabetes * | 20002 | no, yes | Categorized 0/1 |
| | 18 | Myocardial | 20002 | no, yes | Categorized 0/1 |
| | | infarction * | | | |
| | 19 | Angina * | 20002 | no, yes | Categorized 0/1 |
| | 20 | Stroke * | 20002 | no, yes | Categorized 0/1 |
| | 21 | High blood | 20002 | no, yes | Categorized 0/1 |
| | | pressure * | | | |
| | 22 | Hypothyroidism * | 20002 | no, yes | Categorized 0/1 |
| | 23 | Deep-vein | 20002 | no, yes | Categorized 0/1 |
| | | thrombosis * | | | |
| | 24 | High | 20002 | no, yes | Categorized 0/1 |
| | | cholesterol * | | | |
| Respiratory | 25 | Breathing: | 2316 | no, yes | Categorized 0/1 |
| | | wheeze in | | | |
| | | last year | | | |
| | 26 | Pneumonia * | 20002 | no, yes | Categorized 0/1 |
| | 27 | Chronic | 20002 | no, yes | Categorized 0/1 |
| | | bronchitis/ | | | |
| | | emphysema * | | | |
| | 28 | Asthma * | 20002 | no, yes | Categorized 0/1 |
| Musculoskeletal | 29 | Rheumatoid | 20002 | no, yes | Categorized 0/1 |
| | | arthritis * | | | |
| | 30 | Osteoarthritis * | 20002 | no, yes | Categorized 0/1 |
| | 31 | Gout * | 20002 | no, yes | Categorized 0/1 |
| | 32 | Osteoporosis * | 20002 | no, yes | Categorized 0/1 |
| Immunological | 33 | Hay fever, | 20002 | no, yes | Categorized 0/1 |
| | | allergic rhinitis | | | |
| | | or eczema * | | | |
| | 34 | Psoriasis * | 20002 | no, yes | Categorized 0/1 |
| Cancer | 35 | Any cancer | 2453 | no, yes | Categorized 0/1 |
| | | diagnosis * | | | |
| | 36 | Multiple cancers | 134 | Range from 0 | 0—no cancer |
| | | diagnosed | | to 6 | or single cancer, |
| | | (number reported) | | | 1—multiple cancers |
| Pain | 37 | Chest pain | 2335 | no, yes | Categorized 0/1 |
| | 38 | Head and/or neck | 6159 | no, yes | Categorized 0/1 |
| | | pain | | (combining | |
| | | | | responses to | |
| | | | | pain in head | |
| | | | | and neck/ | |
| | | | | shoulders) | |
| | 39 | Back pain | 6159 | no, yes | Categorized 0/1 |
| | 40 | Stomach/ | 6159 | no, yes | Categorized 0/1 |
| | | abdominal pain | | | |
| | 41 | Hip pain | 6159 | no, yes | Categorized 0/1 |
| | 42 | Knee pain | 6159 | no, yes | Categorized 0/1 |
| | 43 | Whole-body pain | 6159 | no, yes | Categorized 0/1 |
| | 44 | Facial pain | 6159 | no, yes | Categorized 0/1 |
| | 45 | Sciatica * | 20002 | no, yes | Categorized 0/1 |
| Gastrointestinal | 46 | Gastric reflux * | 20002 | no, yes | Categorized 0/1 |
| | 47 | Hiatus hernia * | 20002 | no, yes | Categorized 0/1 |
| | 48 | Gall stones * | 20002 | no, yes | Categorized 0/1 |
| | 49 | Diverticulitis * | 20002 | no, yes | Categorized 0/1 |

* Self-reported from the baseline verbal interview. Frailty index was developed by Williams et al. 2019 in the UK Biobank. To create the score, 49 items are coded using the table. The frailty score is calculated by summing all 49 codes and dividing by the total number of items (49).

TABLE 24
Variables used to calculate prevalence and incidence of chronic diseases and
clinical risk factors in the UK Biobank. ICD-9/10 codes and descriptions of
self-report, biochemistry, and clinical interview variables used to code prevalent
and incident disease outcomes. Verbal interview diagnosis codes are contained in the
non-cancer illness (field ID 20002) variables. Incident disease case were mapped to
corresponding ICD codes from the cancer register data (Field IDs 20006, 400013, 40005)
and the HESIN and HESIN_DIAG data tables. For all incident diseases, additional
cases were retrieve using ICD-10 codes from cause of death information from
linked death register data. Baseline prevalence for all diseases and clinical
risk factors was calculated for all participants using baseline measures (including
verbal interview diagnosis codes) + those with an ICD diagnosis before
or on the date of recruitment into the UK Biobank. Incident cases are defined
as those with an ICD date of diagnosis after the date of recruitment who do
not have any prevalent diagnosis. Unless specific ICD subcategories are already
given with dot separators, all ICD codes listed also include all subcategories
(e.g., J44 includes J44, J44.0, J44.1, J44.8, J44.9).

| | | Baseline verbal | | |
|---|---|---|---|---|
| | Baseline | interview | | |
| | measures | diagnosis | ICD-10 | ICD-9 |
| | (field ID) | codes | codes | codes |
| Chronic diseases | | | | |
| Colorectal cancer | — | — | C18-C20 | 153, 154 |
| Lung cancer | — | — | C33, C34 | 162 |
| Esophageal cancer | — | — | C15 | 150 |
| Liver cancer | — | — | C22 | 155 |
| Pancreatic cancer | — | — | C25 | 157 |
| Brain cancer | — | — | C71 | 191 |
| Leukemia | — | — | C91-C95 | 204-208 |
| Non-Hodgkin lymphoma | — | — | C82-C86 | 200, 202 |
| Breast cancer | — | — | C50 | 174 |
| Ovarian cancer | — | — | C56, C57 | 183 |
| Prostate cancer | — | — | C61 | 185 |
| Type 2 diabetes | Taking insulin | 1223 | E11 | 250 |
| | medication | | | |
| | (6153, 6177) | | | |
| | Non-fasting | | | |
| | blood hbA1c ³ | | | |
| | 48 mmol/mol | | | |
| | (30750) | | | |
| | Non-fasting | | | |
| | blood glucose ³ | | | |
| | 11.1 mmol/L | | | |
| | (30740) | | | |
| Ischemic heart disease | — | 1074, 1075 | I20-I25 | 410-414 |
| Cerebrovascular diseases | — | 1081, 1086, | I60-I69 | 430-438 |
| | | 1491, 1583 | | |
| Emphysema, COPD | — | 1112, 1472 | J43-J44 | 492 |
| Chronic liver diseases | — | 1157, 1158, | K70, | 571 |
| | | 1604 | K73-K74, | |
| | | | K75.8, | |
| | | | K76.0 | |
| Chronic kidney diseases | — | 1192, 1193, | N18 | 585 |
| | | 1194 | | |
| All-cause dementia | — | 1263 | A81.0, | 331.0, |
| | | | F00-F03, | 290.4, |
| | | | F05.1, | 331.1, |
| | | | F10.6, | 290.2, |
| | | | G30- | 290.3, |
| | | | G31, | 291.2, |
| | | | I67.3 | 294.1, |
| | | | | 331.2, |
| | | | | 331.5 |
| Vascular dementia | — | 1263 | F01, | 290.4 |
| | | | I67.3 | |
| Alzheimer's disease | — | 1263 | F00, G30 | 331 |
| Parkinson's disease and | — | 1262 | G20-G22 | 332 |
| parkinsonism | | | | |
| Rheumatoid arthritis | — | 1464 | M05-M06 | 714 |
| Macular degeneration | — | 1528 | H35.3 | 362.5 |
| Osteoporosis | | 1309 | M80-M81 | 733 |
| Osteoarthritis | — | 1465 | M15-M19 | 715 |
| Clinical risk factors | | | | |
| Prevalent hypertension | High blood | 1065, 1072 | I10-I15 | 401-405 |
| | pressure | | | |
| | diagnosis by | | | |
| | physician (6150) | | | |
| | Taking | | | |
| | medication for | | | |
| | high blood | | | |
| | pressure (6153, | | | |
| | 6177) | | | |

TABLE 25
Variables used to calculate prevalence and incidence of chronic
diseases and clinical risk factors in the China Kadoorie Biobank.
ICD-10 codes used to code incident disease outcomes. Unless
specific ICD subcategories are already given with dot separators,
all ICD codes listed also include all subcategories (e.g.,
J44 includes J44, J44.0, J44.1, J44.8, J44.9).

| Chronic diseases | ICD-10 codes |
|---|---|
| Ischemic stroke | I63 |
| All stroke | I60-I61, I63-I64 |
| All ischemic heart | I20-I25 |
| disease | |
| Type II diabetes | E11-E14 |
| Chronic obstructive | J41-J44 |
| pulmonary disease | |
| Chronic liver disease | K70, K74-K746 |
| Chronic Kidney disease | N02-N03, N07, |
| | N11, N18 |

### REFERENCES

Akiba, T., Sano, S., Yanase, T., Ohta, T. & Koyama, M. Optuna: A Next-generation Hyperparameter Optimization Framework. KDD '19: Proceedings of the 25th ACM SIGKDD International Conference on Knowledge Discovery & Data Mining, 2623-2631 (2019).
Belsky, D. W. et al. DunedinPACE, a DNA methylation biomarker of the pace of aging. Elife 11 (2022).
Benjamini, Y. & Hochberg, Y. Controlling the False Discovery Rate: A Practical and Powerful Approach to Multiple Testing. Journal of the Royal Statistical Society. Series B 57, 289-300 (1995).
Chen, Z. et al. Cohort profile: the Kadoorie Study of Chronic Disease in China (KSCDC). Int J Epidemiol 34, 1243-1249 (2005).
Chen, Z. et al. China Kadoorie Biobank of 0.5 million people: survey methods, baseline characteristics and long-term follow-up. Int J Epidemiol 40, 1652-1666 (2011).
Codd, V. et al. Measurement and initial characterization of leukocyte telomere length in 474,074 participants in UK Biobank. Nat Aging 2, 170-179 (2022).
Coenen, L., Lehallier, B., de Vries, H. E. & Middeldorp, J. Markers of aging: Unsupervised integrated analyses of the human plasma proteome. Front Aging 4, 1112109 (2023).
Davidson-Pilon, C. lifelines, survival analysis in Python. (2023).
Elliott, P. & Peakman, T. C. The UK Biobank sample handling and storage protocol for the collection, processing and archiving of human blood and urine. International Journal of Epidemiology 37, 234-244 (2008).
Hagberg, A., Schult, A. & Swart, P. in Proceedings of the 7th Python in Science conference (SciPy 2008). (eds G Varoquaux, T Vaught, & J Millman) 11-15.
Horvath, S. DNA methylation age of human tissues and cell types. Genome Biol 14, R115 (2013).
Hunter, J. D. Matplotiib: A 2D Graphics Environment. Computing in Science & Engineering 9, 90-95 (2007).
Johnson, A. A., Shokhirev, M. N., Wyss-Coray, T. & Lehallier, B. Systematic review and analysis of human proteomics aging studies unveils a novel proteomic aging clock and identifies key processes that change with age. Ageing Res Rev 60, 101070 (2020).
Ke, G. et al. LightGBM: A Highly Efficient Gradient Boosting Decision Tree. Advances in Neural Information Processing Systems 30 (NIPS 2017), 3149-3157 (2017).
Kurki, M. I. et al. FinnGen provides genetic insights from a well-phenotyped isolated population. Nature 613, 508-518 (2023).
Kursa, M. B., Jankowski, A. & Rudnicki, W. R. Boruta—A System for Feature Selection. Fundamenta Infornaticae 101, 271-285 (2010).
Lehallier, B. et al. Undulating changes in human plasma proteome profiles across the lifespan. Nat Med 25, 1843-1850 (2019).
Lehallier, B., Shokhirev, M. N., Wyss-Coray, T. & Johnson, A. A. Data mining of human plasma proteins generates a multitude of highly predictive aging clocks that reflect different aspects of aging. Aging Cell 19, e13256 (2020).
Levine, M. E. et al. An epigenetic biomarker of aging for lifespan and healthspan. Aging (Albany NY) 10, 573-591 (2018).
Lundberg, S. M. & Lee, S.-I. A unified approach to interpreting model predictions. Advances in Neural Information Processing Systems 30, 4765-4774 (2017).
Lundberg, S. M. et al. From Local Explanations to Global Understanding with Explainable A1 for Trees. Nat Mach Intell 2, 56-67 (2020).
Macdonald-Dunlop, E. et al. A catalogue of omics biological ageing docks reveals substantial commonality and associations with disease risk. Aging (Albany NY) 14, 623-659 (2022).
Mayer, M. missRanger Fast Imputation of Missing Values. R package version 2.1.0., https://CRAN.R-project.org/package=rmissRanger (2019).
Oh, H. S. et al. Organ aging signatures in the plasma proteome track health and disease. Nature 624, 164-172 (2023).
Palmer, L. UK Biobank: bank on it. Lancet 369, 1980-1982 (2007).
Pollack M M, Holubkov R, Funai T, Dean J M, Berger J T, Wessel D L, Meert K, Berg R A, Newth C J, Harrison R E, Carcillo J, Dalton H, Shanley T, Jenkins T L, Tamburro R; Eunice Kennedy Shriver National Institute of Child Health and Human Development Collaborative Pediatric Critical Care Research Network. The Pediatric Risk of Mortality Score: Update 2015. Pediatr Crit Care Med. (2016)
Rutledge, J., Oh, H. & Wyss-Coray, T. Measuring biological age using omics data. Nat Rev Genet 23, 715-727 (2022).
Sayed, N. et al. An inflammatory aging clock (iAge) based on deep learning tracks multimorbidity, immunosenescence, frailty and cardiovascular aging. Nat Aging 1, 598-615 (2021).
Skipper, S. & Perktold, J. Statsmodels: Econometric and statistical modeling with python. Proceedings of the 9th Python in Science Conference (2010).
Sluiskes, M. H., Goeman, J. J., Beekman, M. et al. Clarifying the biological and statistical assumptions of cross-sectional biological age predictors: an elaborate illustration using synthetic and real data. BMC Med Res Methodol 24, 58 (2024).
Sudlow C, Gallacher J, Allen N, Beral V, Burton P, et al. (2015) UK Biobank: An Open Access Resource for Identifying the Causes of a Wide Range of Complex Diseases of Middle and Old Age. PLOS Medicine 12(3)
Sun, B. B. et al. Plasma proteomic associations with genetics and health in the UK Biobank. Nature 622, 329-338 (2023).
Szklarczyk, D. et al. STRING v10: protein-protein interaction networks, integrated over the tree of life. Nucleic Acids Res 43, D447-452 (2015).
Tanaka, T. et al. Plasma proteomic biomarker signature of age predicts health and life span. Elife 9 (2020).
Waskom, M. L. seaborn: statistical data visualization. Journal of Open Source Software 6, 3021 (2021).
Williams, D. M., Jylhävä, J., Pedersen, N. L. & Hägg, S. A Frailty Index for UK Biobank Participants. J Gerontol A Biol Sci Med Sci 74, 582-587 (2019).
Zimmerman, Jack E. MD, FCCM; Kramer, Andrew A. PhD; McNair, Douglas S. MD, PhD; Malila, Fern M. RN, MS. Acute Physiology and Chronic Health Evaluation (APACHE) IV: Hospital mortality assessment for today's critically ill patients. Critical Care Medicine 34(5):p 1297-1310, (2006)

### CLAUSES OF THE INVENTION

1. A method for determining, predicting or estimating the biological age of a subject, or for providing a measurement for use in determining, predicting or estimating the biological age of a subject, wherein the method comprises:
a) measuring, in a biological sample obtained from the subject at a first time point, the presence or amount of each biomarker in a set of biomarkers, wherein the set of biomarkers comprises at least 7 biomarkers selected from the biomarkers of Table 1:

TABLE 1

| | |
|---|---|
| Acrosomal protein SP-10 | Glial fibrillary acidic protein |
| Agouti-related protein | Immunoglobulin superfamily DCC |
| | subclass member 4 |
| CUB domain-containing protein 1 | Prostate-specific antigen |
| Collagen alpha-3(VI) chain | Kallikrein-7 |
| C-X-C motif chemokine 17 | Leukocyte cell-derived chemotaxin-2 |
| Tumor necrosis factor receptor | Latent-transforming growth factor |
| superfamily member 27 | beta-binding protein 2 |
| Elastin | Neurofilament light polypeptide |
| Endoglin | Podocalyxin-like protein 2 |
| Follitropin subunit beta | Receptor-type tyrosine-protein |
| | phosphatase R |
| Growth/differentiation factor 15 | Scavenger receptor class F member 2 |

2. A method for determining, predicting or estimating the biological age of a subject, or for providing a measurement for use in determining, predicting or estimating the biological age of a subject, wherein the method comprises:
a) measuring, in a biological sample obtained from the subject at a first time point, the presence or amount of each biomarker in a set of biomarkers, wherein the set of biomarkers comprises at least 50 biomarkers selected from the biomarkers of Table 2:

TABLE 2

| | |
|---|---|
| Acrosomal protein SP-10 | PDZ domain-containing protein GIPC2 |
| Actin, aortic smooth muscle | Pancreatic secretory granule membrane |
| | major glycoprotein GP2 |
| Adenosine deaminase | Granzyme B |
| A disintegrin and metalloproteinase with | Hepatitis A virus cellular receptor 1 |
| thrombospondin motifs 13 | |
| A disintegrin and metalloproteinase with | Hemicentin-2 |
| thrombospondin motifs 15 | |
| A disintegrin and metalloproteinase with | Corticosteroid 11-beta-dehydrogenase |
| thrombospondin motifs 16 | isozyme 1 |
| ADAMTS-like protein 5 | Immunoglobulin superfamily DCC |
| | subclass member 4 |
| Adhesion G-protein coupled receptor G1 | Interleukin-17D |
| Alpha-fetoprotein | Interleukin-5 receptor subunit alpha |
| Advanced glycosylation end product- | Interleukin-7 receptor subunit alpha |
| specific receptor | |
| Agouti-related protein | Insulin-like 3 |
| Protein AHNAK2 | Integrin alpha-V |
| Angiopoietin-2 | Integrin beta-5 |
| BAG family molecular chaperone | Integrin beta-like protein 1 |
| regulator 3 | |
| Brevican core protein | Kinesin-like protein KIF22 |
| Osteocalcin | Mast/stem cell growth factor receptor Kit |
| Brother of CDO | Kallikrein-14 |
| Basigin | Prostate-specific antigen |
| Protein C19orf12 | Kallikrein-4 |
| Complement C1q-like protein 2 | Kallikrein-7 |
| Carbonic anhydrase 14 | Kallikrein-8 |
| Carbonic anhydrase 4 | Killer cell lectin-like receptor subfamily F |
| | member 1 |
| Calbindin | Neural cell adhesion molecule L1 |
| Coiled-coil domain-containing protein 80 | Extracellular glycoprotein lacritin |
| C-C motif chemokine 28 | Leukocyte cell-derived chemotaxin-2 |
| CCN family member 5 | Protein LEG1 homolog |
| T-cell surface glycoprotein CD1c | Lutropin subunit beta |
| Endosialin | Leiomodin-1 |
| T-cell surface glycoprotein CD8 alpha | Lactoperoxidase |
| chain | |
| Complement component C1q receptor | Latent-transforming growth factor beta- |
| | binding protein 2 |
| CUB domain-containing protein 1 | Ly6/PLAUR domain-containing protein 3 |
| Cadherin-2 | Apical endosomal glycoprotein |
| Cadherin-3 | Matrilin-3 |
| Cadherin-related family member 2 | Meprin A subunit beta |
| Cell adhesion molecule-related/down- | Matrix extracellular phosphoglycoprotein |
| regulated by oncogenes | |
| Cadherin EGF LAG seven-pass G-type | Tyrosine-protein kinase Mer |
| receptor 2 | |
| Complement factor H-related protein 5 | Lactadherin |
| Secretogranin-1 | Promotilin |
| Chitotriosidase-1 | Macrophage metalloelastase |
| Chordin-like protein 1 | Myelin-oligodendrocyte glycoprotein |
| Chordin-like protein 2 | Matrix remodeling-associated protein 8 |
| Cytoskeleton-associated protein 4 | Neurocan core protein |
| C-type lectin domain family 14 member | Neurofilament light polypeptide |
| A | |
| Contactin-5 | Nucleoside diphosphate kinase 3 |
| Collagen alpha-1(XV) chain | Neurogenic locus notch homolog protein |
| | 3 |
| Collagen alpha-3(VI) chain | N-acetylneuraminate lyase |
| Collagen alpha-1(IX) chain | Neuronal pentraxin-2 |
| Complement receptor type 2 | Neurotrophin-3 |
| Corticoliberin | Neurotrophin-4 |
| Cartilage acidic protein 1 | N-terminal prohormone of brain |
| | natriuretic peptide |
| Beta-crystallin B2 | Odontogenic ameloblast-associated |
| | protein |
| Chondroitin sulfate proteoglycan 5 | Glycodelin |
| Cystatin-SN | Inactive serine protease PAMR1 |
| Cystatin-D | phospholipase A2 inhibitor and |
| | Ly6/PLAUR domain-containing protein |
| Collagen triple helix repeat-containing | Polycystin-1 |
| protein 1 | |
| Cathepsin F | Tissue-type plasminogen activator |
| Cathepsin L2 | Podocalyxin-like protein 2 |
| Coxsackievirus and adenovirus receptor | Pro-opiomelanocortin |
| Stromal cell-derived factor 1 | Prolargin |
| C-X-C motif chemokine 14 | Prolactin |
| C-X-C motif chemokine 17 | Prion-like protein doppel |
| C-X-C motif chemokine 9 | Prokineticin-1 |
| NADH-cytochrome b5 reductase 2 | Persephin |
| Cytokine-like protein 1 | Prostaglandin-H2 D-isomerase |
| Discoidin, CUB and LCCL domain- | Pleiotrophin |
| containing protein 2 | |
| Decorin | Receptor-type tyrosine-protein |
| | phosphatase mu |
| Divergent protein kinase domain 2B | Receptor-type tyrosine-protein |
| | phosphatase N2 |
| Dickkopf-related protein 3 | Receptor-type tyrosine-protein |
| | phosphatase R |
| Dickkopf-like protein 1 | Receptor-type tyrosine-protein |
| | phosphatase zeta |
| Protein delta homolog 1 | Renin |
| Dentin matrix acidic phosphoprotein 1 | Proto-oncogene tyrosine-protein kinase |
| | receptor Ret |
| Dipeptidase 2 | Repulsive guidance molecule A |
| Dermatopontin | RGM domain family member B |
| Tumor necrosis factor receptor | Prorelaxin H2 |
| superfamily member 27 | |
| Epididymal secretory protein E3-beta | Roundabout homolog 1 |
| EGF-like repeat and discoidin I-like | Ribonucleoside-diphosphate reductase |
| domain-containing protein 3 | subunit M2 |
| EGF-containing fibulin-like extracellular | Scavenger receptor class F member 2 |
| matrix protein 1 | |
| EF-hand domain-containing protein D1 | Secretogranin-2 |
| Epidermal growth factor receptor | Secretogranin-3 |
| Elastin | Uteroglobin |
| Protein enabled homolog | Protein sidekick-2 |
| Endoglin | Neuronal-specific septin-3 |
| Beta-enolase | Superoxide dismutase [Mn], |
| | mitochondrial |
| Ectonucleotide | VPS10 domain-containing receptor |
| pyrophosphatase/phosphodiesterase | SorCS2 |
| family member 2 | |
| Ectonucleotide | Sclerostin |
| pyrophosphatase/phosphodiesterase | |
| family member 5 | |
| Receptor tyrosine-protein kinase erbB-4 | Serine protease inhibitor Kazal-type 1 |
| Fatty acid-binding protein, adipocyte | Spondin-2 |
| Protein FAM3B | Small proline-rich protein 3 |
| Prolyl endopeptidase FAP | Sushi repeat-containing protein SRPX |
| Tumor necrosis factor receptor | Sushi domain-containing protein 2 |
| superfamily member 6 | |
| Tumor necrosis factor ligand superfamily | Sushi domain-containing protein 5 |
| member 6 | |
| Fibulin-2 | Trefoil factor 1 |
| Fc receptor-like protein 2 | Thrombospondin-2 |
| Fibroblast growth factor 5 | Tumor necrosis factor receptor |
| | superfamily member 11B |
| Follitropin subunit beta | Tumor necrosis factor receptor |
| | superfamily member 13B |
| Follistatin-related protein 1 | Tumor necrosis factor ligand superfamily |
| | member 13 |
| Growth arrest-specific protein 6 | Tenascin-X |
| Growth/differentiation factor 15 | Tetraspanin-1 |
| Glial fibrillary acidic protein | WAP four-disulfide core domain protein 2 |
| GDNF family receptor alpha-like | Wnt inhibitory factor 1 |
| Appetite-regulating hormone | Protein Wnt-9a |
| Gastric inhibitory polypeptide | Lymphotactin |

3. A method for predicting the presence or absence of at least one disease in a subject, predicting the severity of at least one disease in a subject, predicting the risk of a subject developing at least one disease; and/or predicting the risk of mortality of a subject, wherein the method comprises:
a) measuring, in a biological sample obtained from the subject at a first time point, the presence or amount of each biomarker in a set of biomarkers, wherein the set of biomarkers comprises at least 7 biomarkers selected from the biomarkers of Table 1:

TABLE 1

| | |
|---|---|
| Acrosomal protein SP-10 | Glial fibrillary acidic protein |
| Agouti-related protein | Immunoglobulin superfamily DCC |
| | subclass member 4 |
| CUB domain-containing protein 1 | Prostate-specific antigen |
| Collagen alpha-3(VI) chain | Kallikrein-7 |
| C-X-C motif chemokine 17 | Leukocyte cell-derived chemotaxin-2 |
| Tumor necrosis factor receptor | Latent-transforming growth factor |
| superfamily member 27 | beta-binding protein 2 |
| Elastin | Neurofilament light polypeptide |
| Endoglin | Podocalyxin-like protein 2 |
| Follitropin subunit beta | Receptor-type tyrosine-protein |
| | phosphatase R |
| Growth/differentiation factor 15 | Scavenger receptor class F member 2. |

4. A method for predicting the presence or absence of at least one disease in a subject, predicting the severity of at least one disease in a subject, predicting the risk of a subject developing at least one disease, and/or predicting the risk of mortality of a subject, wherein the method comprises:
a) measuring, in a biological sample obtained from the subject at a first time point, the presence or amount of each biomarker in a set of biomarkers, wherein the set of biomarkers comprises at least 50 biomarkers selected from the biomarkers of Table 2:

TABLE 2

| | |
|---|---|
| Acrosomal protein SP-10 | PDZ domain-containing protein GIPC2 |
| Actin, aortic smooth muscle | Pancreatic secretory granule membrane |
| | major glycoprotein GP2 |
| Adenosine deaminase | Granzyme B |
| A disintegrin and metalloproteinase with | Hepatitis A virus cellular receptor 1 |
| thrombospondin motifs 13 | |
| A disintegrin and metalloproteinase with | Hemicentin-2 |
| thrombospondin motifs 15 | |
| A disintegrin and metalloproteinase with | Corticosteroid 11-beta-dehydrogenase |
| thrombospondin motifs 16 | isozyme 1 |
| ADAMTS-like protein 5 | Immunoglobulin superfamily DCC |
| | subclass member 4 |
| Adhesion G-protein coupled receptor G1 | Interleukin-17D |
| Alpha-fetoprotein | Interleukin-5 receptor subunit alpha |
| Advanced glycosylation end product- | Interleukin-7 receptor subunit alpha |
| specific receptor | |
| Agouti-related protein | Insulin-like 3 |
| Protein AHNAK2 | Integrin alpha-V |
| Angiopoietin-2 | Integrin beta-5 |
| BAG family molecular chaperone | Integrin beta-like protein 1 |
| regulator 3 | |
| Brevican core protein | Kinesin-like protein KIF22 |
| Osteocalcin | Mast/stem cell growth factor receptor Kit |
| Brother of CDO | Kallikrein-14 |
| Basigin | Prostate-specific antigen |
| Protein C19orf12 | Kallikrein-4 |
| Complement C1q-like protein 2 | Kallikrein-7 |
| Carbonic anhydrase 14 | Kallikrein-8 |
| Carbonic anhydrase 4 | Killer cell lectin-like receptor subfamily F |
| | member 1 |
| Calbindin | Neural cell adhesion molecule L1 |
| Coiled-coil domain-containing protein 80 | Extracellular glycoprotein lacritin |
| C-C motif chemokine 28 | Leukocyte cell-derived chemotaxin-2 |
| CCN family member 5 | Protein LEG1 homolog |
| T-cell surface glycoprotein CD1c | Lutropin subunit beta |
| Endosialin | Leiomodin-1 |
| T-cell surface glycoprotein CD8 alpha | Lactoperoxidase |
| chain | |
| Complement component C1q receptor | Latent-transforming growth factor beta- |
| | binding protein 2 |
| CUB domain-containing protein 1 | Ly6/PLAUR domain-containing protein 3 |
| Cadherin-2 | Apical endosomal glycoprotein |
| Cadherin-3 | Matrilin-3 |
| Cadherin-related family member 2 | Meprin A subunit beta |
| Cell adhesion molecule-related/down- | Matrix extracellular phosphoglycoprotein |
| regulated by oncogenes | |
| Cadherin EGF LAG seven-pass G-type | Tyrosine-protein kinase Mer |
| receptor 2 | |
| Complement factor H-related protein 5 | Lactadherin |
| Secretogranin-1 | Promotilin |
| Chitotriosidase-1 | Macrophage metalloelastase |
| Chordin-like protein 1 | Myelin-oligodendrocyte glycoprotein |
| Chordin-like protein 2 | Matrix remodeling-associated protein 8 |
| Cytoskeleton-associated protein 4 | Neurocan core protein |
| C-type lectin domain family 14 member | Neurofilament light polypeptide |
| A | |
| Contactin-5 | Nucleoside diphosphate kinase 3 |
| Collagen alpha-1(XV) chain | Neurogenic locus notch homolog protein |
| | 3 |
| Collagen alpha-3(VI) chain | N-acetylneuraminate lyase |
| Collagen alpha-1(IX) chain | Neuronal pentraxin-2 |
| Complement receptor type 2 | Neurotrophin-3 |
| Corticoliberin | Neurotrophin-4 |
| Cartilage acidic protein 1 | N-terminal prohormone of brain |
| | natriuretic peptide |
| Beta-crystallin B2 | Odontogenic ameloblast-associated |
| | protein |
| Chondroitin sulfate proteoglycan 5 | Glycodelin |
| Cystatin-SN | Inactive serine protease PAMR1 |
| Cystatin-D | phospholipase A2 inhibitor and |
| | Ly6/PLAUR domain-containing protein |
| Collagen triple helix repeat-containing | Polycystin-1 |
| protein 1 | |
| Cathepsin F | Tissue-type plasminogen activator |
| Cathepsin L2 | Podocalyxin-like protein 2 |
| Coxsackievirus and adenovirus receptor | Pro-opiomelanocortin |
| Stromal cell-derived factor 1 | Prolargin |
| C-X-C motif chemokine 14 | Prolactin |
| C-X-C motif chemokine 17 | Prion-like protein doppel |
| C-X-C motif chemokine 9 | Prokineticin-1 |
| NADH-cytochrome b5 reductase 2 | Persephin |
| Cytokine-like protein 1 | Prostaglandin-H2 D-isomerase |
| Discoidin, CUB and LCCL domain- | Pleiotrophin |
| containing protein 2 | |
| Decorin | Receptor-type tyrosine-protein |
| | phosphatase mu |
| Divergent protein kinase domain 2B | Receptor-type tyrosine-protein |
| | phosphatase N2 |
| Dickkopf-related protein 3 | Receptor-type tyrosine-protein |
| | phosphatase R |
| Dickkopf-like protein 1 | Receptor-type tyrosine-protein |
| | phosphatase zeta |
| Protein delta homolog 1 | Renin |
| Dentin matrix acidic phosphoprotein 1 | Proto-oncogene tyrosine-protein kinase |
| | receptor Ret |
| Dipeptidase 2 | Repulsive guidance molecule A |
| Dermatopontin | RGM domain family member B |
| Tumor necrosis factor receptor | Prorelaxin H2 |
| superfamily member 27 | |
| Epididymal secretory protein E3-beta | Roundabout homolog 1 |
| EGF-like repeat and discoidin I-like | Ribonucleoside-diphosphate reductase |
| domain-containing protein 3 | subunit M2 |
| EGF-containing fibulin-like extracellular | Scavenger receptor class F member 2 |
| matrix protein 1 | |
| EF-hand domain-containing protein D1 | Secretogranin-2 |
| Epidermal growth factor receptor | Secretogranin-3 |
| Elastin | Uteroglobin |
| Protein enabled homolog | Protein sidekick-2 |
| Endoglin | Neuronal-specific septin-3 |
| Beta-enolase | Superoxide dismutase [Mn], |
| | mitochondrial |
| Ectonucleotide | VPS10 domain-containing receptor |
| pyrophosphatase/phosphodiesterase | SorCS2 |
| family member 2 | |
| Ectonucleotide | Sclerostin |
| pyrophosphatase/phosphodiesterase | |
| family member 5 | |
| Receptor tyrosine-protein kinase erbB-4 | Serine protease inhibitor Kazal-type 1 |
| Fatty acid-binding protein, adipocyte | Spondin-2 |
| Protein FAM3B | Small proline-rich protein 3 |
| Prolyl endopeptidase FAP | Sushi repeat-containing protein SRPX |
| Tumor necrosis factor receptor | Sushi domain-containing protein 2 |
| superfamily member 6 | |
| Tumor necrosis factor ligand superfamily | Sushi domain-containing protein 5 |
| member 6 | |
| Fibulin-2 | Trefoil factor 1 |
| Fc receptor-like protein 2 | Thrombospondin-2 |
| Fibroblast growth factor 5 | Tumor necrosis factor receptor |
| | superfamily member 11B |
| Follitropin subunit beta | Tumor necrosis factor receptor |
| | superfamily member 13B |
| Follistatin-related protein 1 | Tumor necrosis factor ligand superfamily |
| | member 13 |
| Growth arrest-specific protein 6 | Tenascin-X |
| Growth/differentiation factor 15 | Tetraspanin-1 |
| Glial fibrillary acidic protein | WAP four-disulfide core domain protein 2 |
| GDNF family receptor alpha-like | Wnt inhibitory factor 1 |
| Appetite-regulating hormone | Protein Wnt-9a |
| Gastric inhibitory polypeptide | Lymphotactin |

5. The method of clause 1 or 3, wherein the set of biomarkers comprises at least 8, 9, 10, 11, 12, 13, 14, 15, 16, 17, 18, 19, or 20 biomarkers selected from the biomarkers of Table 1.
6. The method of clause 2 or 4, wherein the set of biomarkers comprises at least 75, 100, 125, 150, 175, 200 or 204 biomarkers selected from the biomarkers of Table 2.
7. The method of any preceding clause, wherein the subject is a human.
8. The method of any preceding clause, wherein the biological sample is a blood-based sample.
9. The method of clause 8, wherein the blood based sample is plasma or serum.
10. The method of any preceding clause, wherein the method further comprises
b) measuring, in a further biological sample obtained from the subject at a different time point from step a), the presence or amount of each biomarker in the set of biomarkers;
c) determining the difference in the presence or amount of each biomarker in the set of biomarkers between the measurements of step a) and step b).
   11. The method of any preceding clause, wherein the method further comprises;
d) comparing the measurement of step a), or the determined difference of step c) with a reference measurement obtained from a subject of a known chronological age to determine, predict or estimate a biological age of the subject.
   12. The method of clause 11, wherein the method further comprises;
e) determining the relationship between chronological age and the biological age of the subject to determine or estimate a value of accelerated or decelerated aging of the subject.
   13. The method of clause 12, wherein a greater chronological age than biological age in the subject indicates decelerated aging of the subject.
   14. The method of clause 12 or 13, wherein a greater biological age than chronological age in the subject indicates accelerated aging of the subject.
   15. The method of any one of clauses 12 to 14, wherein the method further comprises;
f) using the value of accelerated or decelerated aging of the subject to predict:
   i) the presence or absence of at least one disease in the subject;
   ii) the severity of at least one disease in a subject
   iii) the risk of the subject developing at least one disease; and/or
   iv) the risk of mortality of the subject.
      16. The method of any preceding clause, wherein the method further comprises:
g) comparing the measurement of step a), or the determined difference of step c) with reference measurements from a subject with a known disease, known risk of disease, or known risk or mortality to predict;
   i) the presence or absence of at least one disease in the subject;
   ii) the severity of at least one disease in a subject;
   iii) the risk of the subject developing at least one disease; and/or
   iv) the risk of mortality of the subject.
      17. The method of any one of clauses 3, 4, 15 or 16, wherein the at least one disease is an age-related disease.
      18. The method of any one of clauses 3, 4 or 15 to 17, wherein the at least one disease is selected from chronic liver disease, type II diabetes, Parkinson's disease, rheumatoid arthritis, osteoarthritis, macular degeneration, ischemic heart disease, stroke, osteoporosis, ischemic stroke, emphysema, chronic obstructive pulmonary disease (COPD), chronic kidney diseases, all-cause dementia, Alzheimer's disease, oesophageal cancer, prostate cancer, lung cancer, non-Hodgkin lymphoma or combinations thereof.
      19. The method of any one of clauses 3, 4, 15, or 16, wherein mortality is selected from all-cause mortality; age-related mortality; or mortality related to; chronic liver disease, type II diabetes, Parkinson's disease, rheumatoid arthritis, osteoarthritis, macular degeneration, ischemic heart disease, stroke, osteoporosis, ischemic stroke, emphysema, chronic obstructive pulmonary disease (COPD), chronic kidney diseases, all-cause dementia, Alzheimer's disease, oesophageal cancer, prostate cancer, lung cancer, non-Hodgkin lymphoma or combinations thereof.
      20. The method of any preceding clause, wherein the method is an in vitro and/or ex vivo method.
      21. The method of any preceding clause, wherein the biomarkers are proteins, or fragments of proteins.
      22. A device for determining the presence or amount of each biomarker in a set of biomarkers;
wherein the device comprises a set of probes for detection of the biomarkers in the set of biomarkers, wherein the set of probes is specific for and capable of recognising the set of biomarkers in a biological sample from a subject; and
wherein the set of biomarkers comprises at least 7, 8, 9, 10, 11, 12, 13, 14, 15, 16, 17, 18, 19, or 20 proteins selected from the biomarkers of Table 1:

TABLE 1

| | |
|---|---|
| Acrosomal protein SP-10 | Glial fibrillary acidic protein |
| Agouti-related protein | Immunoglobulin superfamily DCC |
| | subclass member 4 |
| CUB domain-containing protein 1 | Prostate-specific antigen |
| Collagen alpha-3(VI) chain | Kallikrein-7 |
| C-X-C motif chemokine 17 | Leukocyte cell-derived chemotaxin-2 |
| Tumor necrosis factor receptor | Latent-transforming growth factor |
| superfamily member 27 | beta-binding protein 2 |
| Elastin | Neurofilament light polypeptide |
| Endoglin | Podocalyxin-like protein 2 |
| Follitropin subunit beta | Receptor-type tyrosine-protein |
| | phosphatase R |
| Growth/differentiation factor 15 | Scavenger receptor class F member 2 |

23. A device for determining the presence or amount of each biomarker in a set of biomarkers,
wherein the device comprises a set of probes for detection of the biomarkers in the set of biomarkers, wherein the set of probes is specific for and capable of recognising the set of biomarkers in a biological sample from a subject; and
wherein the set of biomarkers further comprises at least 50, 75, 100, 125, 150, 175, 200 or 204 biomarkers selected from the biomarkers of Table 2:

TABLE 2

| | |
|---|---|
| Acrosomal protein SP-10 | PDZ domain-containing protein GIPC2 |
| Actin, aortic smooth muscle | Pancreatic secretory granule membrane |
| | major glycoprotein GP2 |
| Adenosine deaminase | Granzyme B |
| A disintegrin and metalloproteinase with | Hepatitis A virus cellular receptor 1 |
| thrombospondin motifs 13 | |
| A disintegrin and metalloproteinase with | Hemicentin-2 |
| thrombospondin motifs 15 | |
| A disintegrin and metalloproteinase with | Corticosteroid 11-beta-dehydrogenase |
| thrombospondin motifs 16 | isozyme 1 |
| ADAMTS-like protein 5 | Immunoglobulin superfamily DCC |
| | subclass member 4 |
| Adhesion G-protein coupled receptor G1 | Interleukin-17D |
| Alpha-fetoprotein | Interleukin-5 receptor subunit alpha |
| Advanced glycosylation end product- | Interleukin-7 receptor subunit alpha |
| specific receptor | |
| Agouti-related protein | Insulin-like 3 |
| Protein AHNAK2 | Integrin alpha-V |
| Angiopoietin-2 | Integrin beta-5 |
| BAG family molecular chaperone | Integrin beta-like protein 1 |
| regulator 3 | |
| Brevican core protein | Kinesin-like protein KIF22 |
| Osteocalcin | Mast/stem cell growth factor receptor Kit |
| Brother of CDO | Kallikrein-14 |
| Basigin | Prostate-specific antigen |
| Protein C19orf12 | Kallikrein-4 |
| Complement C1q-like protein 2 | Kallikrein-7 |
| Carbonic anhydrase 14 | Kallikrein-8 |
| Carbonic anhydrase 4 | Killer cell lectin-like receptor subfamily F |
| | member 1 |
| Calbindin | Neural cell adhesion molecule L1 |
| Coiled-coil domain-containing protein 80 | Extracellular glycoprotein lacritin |
| C-C motif chemokine 28 | Leukocyte cell-derived chemotaxin-2 |
| CCN family member 5 | Protein LEG1 homolog |
| T-cell surface glycoprotein CD1c | Lutropin subunit beta |
| Endosialin | Leiomodin-1 |
| T-cell surface glycoprotein CD8 alpha | Lactoperoxidase |
| chain | |
| Complement component C1q receptor | Latent-transforming growth factor beta- |
| | binding protein 2 |
| CUB domain-containing protein 1 | Ly6/PLAUR domain-containing protein 3 |
| Cadherin-2 | Apical endosomal glycoprotein |
| Cadherin-3 | Matrilin-3 |
| Cadherin-related family member 2 | Meprin A subunit beta |
| Cell adhesion molecule-related/down- | Matrix extracellular phosphoglycoprotein |
| regulated by oncogenes | |
| Cadherin EGF LAG seven-pass G-type | Tyrosine-protein kinase Mer |
| receptor 2 | |
| Complement factor H-related protein 5 | Lactadherin |
| Secretogranin-1 | Promotilin |
| Chitotriosidase-1 | Macrophage metalloelastase |
| Chordin-like protein 1 | Myelin-oligodendrocyte glycoprotein |
| Chordin-like protein 2 | Matrix remodeling-associated protein 8 |
| Cytoskeleton-associated protein 4 | Neurocan core protein |
| C-type lectin domain family 14 member | Neurofilament light polypeptide |
| A | |
| Contactin-5 | Nucleoside diphosphate kinase 3 |
| Collagen alpha-1(XV) chain | Neurogenic locus notch homolog protein |
| | 3 |
| Collagen alpha-3(VI) chain | N-acetylneuraminate lyase |
| Collagen alpha-1(IX) chain | Neuronal pentraxin-2 |
| Complement receptor type 2 | Neurotrophin-3 |
| Corticoliberin | Neurotrophin-4 |
| Cartilage acidic protein 1 | N-terminal prohormone of brain |
| | natriuretic peptide |
| Beta-crystallin B2 | Odontogenic ameloblast-associated |
| | protein |
| Chondroitin sulfate proteoglycan 5 | Glycodelin |
| Cystatin-SN | Inactive serine protease PAMR1 |
| Cystatin-D | phospholipase A2 inhibitor and |
| | Ly6/PLAUR domain-containing protein |
| Collagen triple helix repeat-containing | Polycystin-1 |
| protein 1 | |
| Cathepsin F | Tissue-type plasminogen activator |
| Cathepsin L2 | Podocalyxin-like protein 2 |
| Coxsackievirus and adenovirus receptor | Pro-opiomelanocortin |
| Stromal cell-derived factor 1 | Prolargin |
| C-X-C motif chemokine 14 | Prolactin |
| C-X-C motif chemokine 17 | Prion-like protein doppel |
| C-X-C motif chemokine 9 | Prokineticin-1 |
| NADH-cytochrome b5 reductase 2 | Persephin |
| Cytokine-like protein 1 | Prostaglandin-H2 D-isomerase |
| Discoidin, CUB and LCCL domain- | Pleiotrophin |
| containing protein 2 | |
| Decorin | Receptor-type tyrosine-protein |
| | phosphatase mu |
| Divergent protein kinase domain 2B | Receptor-type tyrosine-protein |
| | phosphatase N2 |
| Dickkopf-related protein 3 | Receptor-type tyrosine-protein |
| | phosphatase R |
| Dickkopf-like protein 1 | Receptor-type tyrosine-protein |
| | phosphatase zeta |
| Protein delta homolog 1 | Renin |
| Dentin matrix acidic phosphoprotein 1 | Proto-oncogene tyrosine-protein kinase |
| | receptor Ret |
| Dipeptidase 2 | Repulsive guidance molecule A |
| Dermatopontin | RGM domain family member B |
| Tumor necrosis factor receptor | Prorelaxin H2 |
| superfamily member 27 | |
| Epididymal secretory protein E3-beta | Roundabout homolog 1 |
| EGF-like repeat and discoidin I-like | Ribonucleoside-diphosphate reductase |
| domain-containing protein 3 | subunit M2 |
| EGF-containing fibulin-like extracellular | Scavenger receptor class F member 2 |
| matrix protein 1 | |
| EF-hand domain-containing protein D1 | Secretogranin-2 |
| Epidermal growth factor receptor | Secretogranin-3 |
| Elastin | Uteroglobin |
| Protein enabled homolog | Protein sidekick-2 |
| Endoglin | Neuronal-specific septin-3 |
| Beta-enolase | Superoxide dismutase [Mn], |
| | mitochondrial |
| Ectonucleotide | VPS10 domain-containing receptor |
| pyrophosphatase/phosphodiesterase | SorCS2 |
| family member 2 | |
| Ectonucleotide | Sclerostin |
| pyrophosphatase/phosphodiesterase | |
| family member 5 | |
| Receptor tyrosine-protein kinase erbB-4 | Serine protease inhibitor Kazal-type 1 |
| Fatty acid-binding protein, adipocyte | Spondin-2 |
| Protein FAM3B | Small proline-rich protein 3 |
| Prolyl endopeptidase FAP | Sushi repeat-containing protein SRPX |
| Tumor necrosis factor receptor | Sushi domain-containing protein 2 |
| superfamily member 6 | |
| Tumor necrosis factor ligand superfamily | Sushi domain-containing protein 5 |
| member 6 | |
| Fibulin-2 | Trefoil factor 1 |
| Fc receptor-like protein 2 | Thrombospondin-2 |
| Fibroblast growth factor 5 | Tumor necrosis factor receptor |
| | superfamily member 11B |
| Follitropin subunit beta | Tumor necrosis factor receptor |
| | superfamily member 13B |
| Follistatin-related protein 1 | Tumor necrosis factor ligand superfamily |
| | member 13 |
| Growth arrest-specific protein 6 | Tenascin-X |
| Growth/differentiation factor 15 | Tetraspanin-1 |
| Glial fibrillary acidic protein | WAP four-disulfide core domain protein 2 |
| GDNF family receptor alpha-like | Wnt inhibitory factor 1 |
| Appetite-regulating hormone | Protein Wnt-9a |
| Gastric inhibitory polypeptide | Lymphotactin |

24. The device of clause 22 or 23, wherein the subject is a human.
25. The device of any one of clauses 22 to 24, wherein biological sample is a blood-based sample.
26. The device of clause 25, wherein the blood-based sample is plasma or serum.
27. The device of any one of clauses 22 to 26, wherein each probe is selected from an antibody, antibody fragment, oligonucleotide, protein, biotin-binding protein, enzyme, fluorophore or combinations thereof.
28. The device of any one of clauses 22 to 27, wherein the biomarkers are proteins, or a fragment of proteins.
29. A set of probes for determining the presence or amount of a set of biomarkers, wherein each probe in the set of probes specifically recognises at least one biomarker in the set of biomarkers; and
wherein the set of biomarkers comprises at least 7, 8, 9, 10, 11, 12, 13, 14, 15, 16, 17, 18, 19, or 20 biomarkers selected from the biomarkers of Table 1:

TABLE 1

| | |
|---|---|
| Acrosomal protein SP-10 | Glial fibrillary acidic protein |
| Agouti-related protein | Immunoglobulin superfamily DCC |
| | subclass member 4 |
| CUB domain-containing protein 1 | Prostate-specific antigen |
| Collagen alpha-3(VI) chain | Kallikrein-7 |
| C-X-C motif chemokine 17 | Leukocyte cell-derived chemotaxin-2 |
| Tumor necrosis factor receptor | Latent-transforming growth factor |
| superfamily member 27 | beta-binding protein 2 |
| Elastin | Neurofilament light polypeptide |
| Endoglin | Podocalyxin-like protein 2 |
| Follitropin subunit beta | Receptor-type tyrosine-protein |
| | phosphatase R |
| Growth/differentiation factor 15 | Scavenger receptor class F member 2 |

30. A set of probes for determining the presence or amount of a set of biomarkers, wherein each probe in the set of probes specifically recognises at least one biomarker in the set of biomarkers; and
wherein the set of biomarkers comprises at least 50, 75, 100, 125, 150, 175, 200 or 204 biomarkers selected from the biomarkers of Table 2:

TABLE 2

| | |
|---|---|
| Acrosomal protein SP-10 | PDZ domain-containing protein GIPC2 |
| Actin, aortic smooth muscle | Pancreatic secretory granule membrane |
| | major glycoprotein GP2 |
| Adenosine deaminase | Granzyme B |
| A disintegrin and metalloproteinase with | Hepatitis A virus cellular receptor 1 |
| thrombospondin motifs 13 | |
| A disintegrin and metalloproteinase with | Hemicentin-2 |
| thrombospondin motifs 15 | |
| A disintegrin and metalloproteinase with | Corticosteroid 11-beta-dehydrogenase |
| thrombospondin motifs 16 | isozyme 1 |
| ADAMTS-like protein 5 | Immunoglobulin superfamily DCC |
| | subclass member 4 |
| Adhesion G-protein coupled receptor G1 | Interleukin-17D |
| Alpha-fetoprotein | Interleukin-5 receptor subunit alpha |
| Advanced glycosylation end product- | Interleukin-7 receptor subunit alpha |
| specific receptor | |
| Agouti-related protein | Insulin-like 3 |
| Protein AHNAK2 | Integrin alpha-V |
| Angiopoietin-2 | Integrin beta-5 |
| BAG family molecular chaperone | Integrin beta-like protein 1 |
| regulator 3 | |
| Brevican core protein | Kinesin-like protein KIF22 |
| Osteocalcin | Mast/stem cell growth factor receptor Kit |
| Brother of CDO | Kallikrein-14 |
| Basigin | Prostate-specific antigen |
| Protein C19orf12 | Kallikrein-4 |
| Complement C1q-like protein 2 | Kallikrein-7 |
| Carbonic anhydrase 14 | Kallikrein-8 |
| Carbonic anhydrase 4 | Killer cell lectin-like receptor subfamily F |
| | member 1 |
| Calbindin | Neural cell adhesion molecule L1 |
| Coiled-coil domain-containing protein 80 | Extracellular glycoprotein lacritin |
| C-C motif chemokine 28 | Leukocyte cell-derived chemotaxin-2 |
| CCN family member 5 | Protein LEG1 homolog |
| T-cell surface glycoprotein CD1c | Lutropin subunit beta |
| Endosialin | Leiomodin-1 |
| T-cell surface glycoprotein CD8 alpha | Lactoperoxidase |
| chain | |
| Complement component C1q receptor | Latent-transforming growth factor beta- |
| | binding protein 2 |
| CUB domain-containing protein 1 | Ly6/PLAUR domain-containing protein 3 |
| Cadherin-2 | Apical endosomal glycoprotein |
| Cadherin-3 | Matrilin-3 |
| Cadherin-related family member 2 | Meprin A subunit beta |
| Cell adhesion molecule-related/down- | Matrix extracellular phosphoglycoprotein |
| regulated by oncogenes | |
| Cadherin EGF LAG seven-pass G-type | Tyrosine-protein kinase Mer |
| receptor 2 | |
| Complement factor H-related protein 5 | Lactadherin |
| Secretogranin-1 | Promotilin |
| Chitotriosidase-1 | Macrophage metalloelastase |
| Chordin-like protein 1 | Myelin-oligodendrocyte glycoprotein |
| Chordin-like protein 2 | Matrix remodeling-associated protein 8 |
| Cytoskeleton-associated protein 4 | Neurocan core protein |
| C-type lectin domain family 14 member | Neurofilament light polypeptide |
| A | |
| Contactin-5 | Nucleoside diphosphate kinase 3 |
| Collagen alpha-1(XV) chain | Neurogenic locus notch homolog protein |
| | 3 |
| Collagen alpha-3(VI) chain | N-acetylneuraminate lyase |
| Collagen alpha-1(IX) chain | Neuronal pentraxin-2 |
| Complement receptor type 2 | Neurotrophin-3 |
| Corticoliberin | Neurotrophin-4 |
| Cartilage acidic protein 1 | N-terminal prohormone of brain |
| | natriuretic peptide |
| Beta-crystallin B2 | Odontogenic ameloblast-associated |
| | protein |
| Chondroitin sulfate proteoglycan 5 | Glycodelin |
| Cystatin-SN | Inactive serine protease PAMR1 |
| Cystatin-D | phospholipase A2 inhibitor and |
| | Ly6/PLAUR domain-containing protein |
| Collagen triple helix repeat-containing | Polycystin-1 |
| protein 1 | |
| Cathepsin F | Tissue-type plasminogen activator |
| Cathepsin L2 | Podocalyxin-like protein 2 |
| Coxsackievirus and adenovirus receptor | Pro-opiomelanocortin |
| Stromal cell-derived factor 1 | Prolargin |
| C-X-C motif chemokine 14 | Prolactin |
| C-X-C motif chemokine 17 | Prion-like protein doppel |
| C-X-C motif chemokine 9 | Prokineticin-1 |
| NADH-cytochrome b5 reductase 2 | Persephin |
| Cytokine-like protein 1 | Prostaglandin-H2 D-isomerase |
| Discoidin, CUB and LCCL domain- | Pleiotrophin |
| containing protein 2 | |
| Decorin | Receptor-type tyrosine-protein |
| | phosphatase mu |
| Divergent protein kinase domain 2B | Receptor-type tyrosine-protein |
| | phosphatase N2 |
| Dickkopf-related protein 3 | Receptor-type tyrosine-protein |
| | phosphatase R |
| Dickkopf-like protein 1 | Receptor-type tyrosine-protein |
| | phosphatase zeta |
| Protein delta homolog 1 | Renin |
| Dentin matrix acidic phosphoprotein 1 | Proto-oncogene tyrosine-protein kinase |
| | receptor Ret |
| Dipeptidase 2 | Repulsive guidance molecule A |
| Dermatopontin | RGM domain family member B |
| Tumor necrosis factor receptor | Prorelaxin H2 |
| superfamily member 27 | |
| Epididymal secretory protein E3-beta | Roundabout homolog 1 |
| EGF-like repeat and discoidin I-like | Ribonucleoside-diphosphate reductase |
| domain-containing protein 3 | subunit M2 |
| EGF-containing fibulin-like extracellular | Scavenger receptor class F member 2 |
| matrix protein 1 | |
| EF-hand domain-containing protein D1 | Secretogranin-2 |
| Epidermal growth factor receptor | Secretogranin-3 |
| Elastin | Uteroglobin |
| Protein enabled homolog | Protein sidekick-2 |
| Endoglin | Neuronal-specific septin-3 |
| Beta-enolase | Superoxide dismutase [Mn], |
| | mitochondrial |
| Ectonucleotide | VPS10 domain-containing receptor |
| pyrophosphatase/phosphodiesterase | SorCS2 |
| family member 2 | |
| Ectonucleotide | Sclerostin |
| pyrophosphatase/phosphodiesterase | |
| family member 5 | |
| Receptor tyrosine-protein kinase erbB-4 | Serine protease inhibitor Kazal-type 1 |
| Fatty acid-binding protein, adipocyte | Spondin-2 |
| Protein FAM3B | Small proline-rich protein 3 |
| Prolyl endopeptidase FAP | Sushi repeat-containing protein SRPX |
| Tumor necrosis factor receptor | Sushi domain-containing protein 2 |
| superfamily member 6 | |
| Tumor necrosis factor ligand superfamily | Sushi domain-containing protein 5 |
| member 6 | |
| Fibulin-2 | Trefoil factor 1 |
| Fc receptor-like protein 2 | Thrombospondin-2 |
| Fibroblast growth factor 5 | Tumor necrosis factor receptor |
| | superfamily member 11B |
| Follitropin subunit beta | Tumor necrosis factor receptor |
| | superfamily member 13B |
| Follistatin-related protein 1 | Tumor necrosis factor ligand superfamily |
| | member 13 |
| Growth arrest-specific protein 6 | Tenascin-X |
| Growth/differentiation factor 15 | Tetraspanin-1 |
| Glial fibrillary acidic protein | WAP four-disulfide core domain protein 2 |
| GDNF family receptor alpha-like | Wnt inhibitory factor 1 |
| Appetite-regulating hormone | Protein Wnt-9a |
| Gastric inhibitory polypeptide | Lymphotactin |

31. The set of probes of clause 29 or 30, wherein each probe in the set is selected from an antibody, antibody fragment, oligonucleotide, protein, biotin-binding protein, enzyme, fluorophore or combination thereof.
32. The set of probes of any one of clauses 29 to 31, wherein the biomarkers a proteins, or a fragment of proteins.
33. The method of any one of clauses 1, 3, 5, or 7 to 21, the device of any one of clauses 22, or 24 to 27 or the set of probes of clauses 29 or 32, wherein the set of biomarkers comprises at least 7, 8, 9 or 10 biomarkers selected from the biomarkers of Table 3:

TABLE 3

| | |
|---|---|
| Tumor necrosis factor receptor | Elastin |
| superfamily member 27 | |
| Collagen alpha-3(VI) chain | Immunoglobulin superfamily DCC |
| | subclass member 4 |
| Growth/differentiation factor 15 | Follitropin subunit beta |
| Neurofilament light polypeptide | Latent-transforming growth factor beta- |
| | binding protein 2 |
| Podocalyxin-like protein 2 | Prostate-specific antigen |

34. A biomarker testing kit comprising a blood sampling device and the set of probes of any one of clauses 29 to 33.
35. The biomarker testing kit of clause 34, wherein the blood sampling device is a patch-based blood sampling device or a finger prick blood sampling device.
36. The use of the device as disclosed in of any one of clauses 23 to 28, the probes as disclosed in any one of clauses 29 to 32 or the biomarker testing kit of clause 34 or 35; in the method as discloses in any one of clauses 1 to 21.
37. A computer-implemented method for determining, predicting or estimating the biological age of a subject comprising the steps of:
a) Obtaining data of the measured levels of: i) at least 7 biomarkers in Table 1; or ii) at least 50 biomarkers in Table 2;
b) Inputting the measured levels in step a) to a predictive model which relates the measured levels with biological age or chronological age; and
c) Outputting a determined, predicted or estimated biological age.
   38. A computer-implemented method for predicting the presence or absence of at least one disease in a subject, predicting the risk of a subject developing at least one disease, and/or predicting the risk of mortality of a subject, wherein the method comprises:
a) Obtaining data of the measured levels of: i) at least 7 biomarkers in Table 1; or ii) at least 50 biomarkers in Table 2;
b) Inputting the measured levels in step a) to a predictive model which relates the measured levels with disease and/or mortality; and
c) Outputting at least one of:
   i) the presence or absence of at least one disease in the subject;
   ii) the severity of at least one disease in a subject;
   iii) the risk of the subject developing at least one disease; and/or
   iv) the risk of mortality of the subject.
      39. A computer-readable storage medium or a computer program comprising computer-executable instructions, which when executed by a computing system, are capable of causing the computing system to perform the method according to clauses 37-38.

What is claimed is:

1. A method for determining, predicting or estimating the biological age of a subject, for providing a measurement for use in determining, predicting or estimating the biological age of a subject, for predicting the presence or absence of at least one disease in a subject, predicting the severity of at least one disease in a subject, predicting the risk of a subject developing at least one disease; and/or predicting the risk of mortality of a subject
wherein the method comprises a) measuring, in a biological sample obtained from the subject at a first time point, the presence or amount of each biomarker in a set of biomarkers, wherein the set of biomarkers comprises
i) at least 7 biomarkers selected from Table 1:
Or
TABLE 1

| | |
|---|---|
| Acrosomal protein SP-10 | Glial fibrillary acidic protein |
| Agouti-related protein | Immunoglobulin superfamily DCC |
| | subclass member 4 |
| CUB domain-containing protein 1 | Prostate-specific antigen |
| Collagen alpha-3(VI) chain | Kallikrein-7 |
| C-X-C motif chemokine 17 | Leukocyte cell-derived chemotaxin-2 |
| Tumor necrosis factor receptor | Latent-transforming growth factor |
| superfamily member 27 | beta-binding protein 2 |
| Elastin | Neurofilament light polypeptide |
| Endoglin | Podocalyxin-like protein 2 |
| Follitropin subunit beta | Receptor-type tyrosine-protein |
| | phosphatase R |
| Growth/differentiation factor 15 | Scavenger receptor class F member 2 |

ii) at least 50 biomarkers selected from Table 2:
TABLE 2

| | |
|---|---|
| Acrosomal protein SP-10 | PDZ domain-containing protein GIPC2 |
| Actin, aortic smooth muscle | Pancreatic secretory granule |
| | membrane major glycoprotein GP2 |
| Adenosine deaminase | Granzyme B |
| A disintegrin and metalloproteinase | Hepatitis A virus cellular receptor 1 |
| with thrombospondin motifs 13 | |
| A disintegrin and metalloproteinase | Hemicentin-2 |
| with thrombospondin motifs 15 | |
| A disintegrin and metalloproteinase | Corticosteroid 11-beta-dehydrogenase |
| with thrombospondin motifs 16 | isozyme 1 |
| ADAMTS-like protein 5 | Immunoglobulin superfamily DCC |
| | subclass member 4 |
| Adhesion G-protein coupled receptor | Interleukin-17D |
| G1 | |
| Alpha-fetoprotein | Interleukin-5 receptor subunit alpha |
| Advanced glycosylation end product- | Interleukin-7 receptor subunit alpha |
| specific receptor | |
| Agouti-related protein | Insulin-like 3 |
| Protein AHNAK2 | Integrin alpha-V |
| Angiopoietin-2 | Integrin beta-5 |
| BAG family molecular chaperone | Integrin beta-like protein 1 |
| regulator 3 | |
| Brevican core protein | Kinesin-like protein KIF22 |
| Osteocalcin | Mast/stem cell growth factor receptor |
| | Kit |
| Brother of CDO | Kallikrein-14 |
| Basigin | Prostate-specific antigen |
| Protein C19orf12 | Kallikrein-4 |
| Complement C1q-like protein 2 | Kallikrein-7 |
| Carbonic anhydrase 14 | Kallikrein-8 |
| Carbonic anhydrase 4 | Killer cell lectin-like receptor subfamily |
| | F member 1 |
| Calbindin | Neural cell adhesion molecule L1 |
| Coiled-coil domain-containing protein | Extracellular glycoprotein lacritin |
| 80 | |
| C-C motif chemokine 28 | Leukocyte cell-derived chemotaxin-2 |
| CCN family member 5 | Protein LEG1 homolog |
| T-cell surface glycoprotein CD1c | Lutropin subunit beta |
| Endosialin | Leiomodin-1 |
| T-cell surface glycoprotein CD8 alpha | Lactoperoxidase |
| chain | |
| Complement component C1q receptor | Latent-transforming growth factor beta- |
| | binding protein 2 |
| CUB domain-containing protein 1 | Ly6/PLAUR domain-containing protein |
| | 3 |
| Cadherin-2 | Apical endosomal glycoprotein |
| Cadherin-3 | Matrilin-3 |
| Cadherin-related family member 2 | Meprin A subunit beta |
| Cell adhesion molecule-related/down- | Matrix extracellular |
| regulated by oncogenes | phosphoglycoprotein |
| Cadherin EGF LAG seven-pass G-type | Tyrosine-protein kinase Mer |
| receptor 2 | |
| Complement factor H-related protein 5 | Lactadherin |
| Secretogranin-1 | Promotilin |
| Chitotriosidase-1 | Macrophage metalloelastase |
| Chordin-like protein 1 | Myelin-oligodendrocyte glycoprotein |
| Chordin-like protein 2 | Matrix remodeling-associated protein 8 |
| Cytoskeleton-associated protein 4 | Neurocan core protein |
| C-type lectin domain family 14 member | Neurofilament light polypeptide |
| A | |
| Contactin-5 | Nucleoside diphosphate kinase 3 |
| Collagen alpha-1(XV) chain | Neurogenic locus notch homolog |
| | protein 3 |
| Collagen alpha-3(VI) chain | N-acetylneuraminate lyase |
| Collagen alpha-1(IX) chain | Neuronal pentraxin-2 |
| Complement receptor type 2 | Neurotrophin-3 |
| Corticoliberin | Neurotrophin-4 |
| Cartilage acidic protein 1 | N-terminal prohormone of brain |
| | natriuretic peptide |
| Beta-crystallin B2 | Odontogenic ameloblast-associated |
| | protein |
| Chondroitin sulfate proteoglycan 5 | Glycodelin |
| Cystatin-SN | Inactive serine protease PAMR1 |
| Cystatin-D | phospholipase A2 inhibitor and |
| | Ly6/PLAUR domain-containing protein |
| Collagen triple helix repeat-containing | Polycystin-1 |
| protein 1 | |
| Cathepsin F | Tissue-type plasminogen activator |
| Cathepsin L2 | Podocalyxin-like protein 2 |
| Coxsackievirus and adenovirus | Pro-opiomelanocortin |
| receptor | |
| Stromal cell-derived factor 1 | Prolargin |
| C-X-C motif chemokine 14 | Prolactin |
| C-X-C motif chemokine 17 | Prion-like protein doppel |
| C-X-C motif chemokine 9 | Prokineticin-1 |
| NADH-cytochrome b5 reductase 2 | Persephin |
| Cytokine-like protein 1 | Prostaglandin-H2 D-isomerase |
| Discoidin, CUB and LCCL domain- | Pleiotrophin |
| containing protein 2 | |
| Decorin | Receptor-type tyrosine-protein |
| | phosphatase mu |
| Divergent protein kinase domain 2B | Receptor-type tyrosine-protein |
| | phosphatase N2 |
| Dickkopf-related protein 3 | Receptor-type tyrosine-protein |
| | phosphatase R |
| Dickkopf-like protein 1 | Receptor-type tyrosine-protein |
| | phosphatase zeta |
| Protein delta homolog 1 | Renin |
| Dentin matrix acidic phosphoprotein 1 | Proto-oncogene tyrosine-protein |
| | kinase receptor Ret |
| Dipeptidase 2 | Repulsive guidance molecule A |
| Dermatopontin | RGM domain family member B |
| Tumor necrosis factor receptor | Prorelaxin H2 |
| superfamily member 27 | |
| Epididymal secretory protein E3-beta | Roundabout homolog 1 |
| EGF-like repeat and discoidin I-like | Ribonucleoside-diphosphate reductase |
| domain-containing protein 3 | subunit M2 |
| EGF-containing fibulin-like extracellular | Scavenger receptor class F member 2 |
| matrix protein 1 | |
| EF-hand domain-containing protein D1 | Secretogranin-2 |
| Epidermal growth factor receptor | Secretogranin-3 |
| Elastin | Uteroglobin |
| Protein enabled homolog | Protein sidekick-2 |
| Endoglin | Neuronal-specific septin-3 |
| Beta-enolase | Superoxide dismutase [Mn], |
| | mitochondrial |
| Ectonucleotide | VPS10 domain-containing receptor |
| pyrophosphatase/phosphodiesterase | SorCS2 |
| family member 2 | |
| Ectonucleotide | Sclerostin |
| pyrophosphatase/phosphodiesterase | |
| family member 5 | |
| Receptor tyrosine-protein kinase erbB- | Serine protease inhibitor Kazal-type 1 |
| 4 | |
| Fatty acid-binding protein, adipocyte | Spondin-2 |
| Protein FAM3B | Small proline-rich protein 3 |
| Prolyl endopeptidase FAP | Sushi repeat-containing protein SRPX |
| Tumor necrosis factor receptor | Sushi domain-containing protein 2 |
| superfamily member 6 | |
| Tumor necrosis factor ligand | Sushi domain-containing protein 5 |
| superfamily member 6 | |
| Fibulin-2 | Trefoil factor 1 |
| Fc receptor-like protein 2 | Thrombospondin-2 |
| Fibroblast growth factor 5 | Tumor necrosis factor receptor |
| | superfamily member 11B |
| Follitropin subunit beta | Tumor necrosis factor receptor |
| | superfamily member 13B |
| Follistatin-related protein 1 | Tumor necrosis factor ligand |
| | superfamily member 13 |
| Growth arrest-specific protein 6 | Tenascin-X |
| Growth/differentiation factor 15 | Tetraspanin-1 |
| Glial fibrillary acidic protein | WAP four-disulfide core domain protein |
| | 2 |
| GDNF family receptor alpha-like | Wnt inhibitory factor 1 |
| Appetite-regulating hormone | Protein Wnt-9a |
| Gastric inhibitory polypeptide | Lymphotactin |

2. The method of claim 1, wherein the set of biomarkers comprises at least 8, 9, 10, 11, 12, 13, 14, 15, 16, 17, 18, 19, or 20 biomarkers selected from Table 1 or at least 75, 100, 125, 150, 175, 200 or 204 biomarkers selected from Table 2.

3. The method of claim 1, wherein the subject is a human.

4. The method of claim 1, wherein the biological sample is a blood-based sample, optionally plasma or serum.

5. The method of claim 1, wherein the method further comprises
b) measuring, in a further biological sample obtained from the subject at a different time point from step a), the presence or amount of each biomarker in the set of biomarkers;
c) determining the difference in the presence or amount of each biomarker in the set of biomarkers between the measurements of step a) and step b);
and optionally
d) comparing the measurement of step a), or the determined difference of step c) with a reference measurement obtained from a subject of a known chronological age to determine, predict or estimate a biological age of the subject.

6. The method of claim 5, wherein the method further comprises;
e) determining the relationship between chronological age and the biological age of the subject to determine or estimate a value of accelerated or decelerated aging of the subject,
optionally wherein the method further comprises;
f) using the value of accelerated or decelerated aging of the subject to predict:
i) the presence or absence of at least one disease in the subject;
ii) the severity of at least one disease in a subject
iii) the risk of the subject developing at least one disease; and/or
iv) the risk of mortality of the subject.

7. The method of claim 5, wherein a greater chronological age than biological age in the subject indicates decelerated aging of the subject or wherein a greater biological age than chronological age in the subject indicates accelerated aging of the subject.

8. The method of claim 5, wherein the method further comprises:
g) comparing the measurement of step a), or the determined difference of step c) with reference measurements from a subject with a known disease, known risk of disease, or known risk or mortality to predict;
i) the presence or absence of at least one disease in the subject;
ii) the severity of at least one disease in a subject;
iii) the risk of the subject developing at least one disease; and/or
iv) the risk of mortality of the subject.

9. The method of claim 1, wherein the at least one disease is an age-related disease, optionally wherein the at least one disease is selected from chronic liver disease, type II diabetes, Parkinson's disease, rheumatoid arthritis, osteoarthritis, macular degeneration, ischemic heart disease, stroke, osteoporosis, ischemic stroke, emphysema, chronic obstructive pulmonary disease (COPD), chronic kidney diseases, all-cause dementia, Alzheimer's disease, oesophageal cancer, prostate cancer, lung cancer, non-Hodgkin lymphoma or combinations thereof.

10. The method of claim 1, wherein mortality is selected from all-cause mortality; age-related mortality; or mortality related to; chronic liver disease, type II diabetes, Parkinson's disease, rheumatoid arthritis, osteoarthritis, macular degeneration, ischemic heart disease, stroke, osteoporosis, ischemic stroke, emphysema, chronic obstructive pulmonary disease (COPD), chronic kidney diseases, all-cause dementia, Alzheimer's disease, oesophageal cancer, prostate cancer, lung cancer, non-Hodgkin lymphoma or combinations thereof.

11. The method of claim 1, wherein one or more of the biomarkers are proteins, or fragments of proteins.

12. A set of probes for determining the presence or amount of a set of biomarkers, wherein each probe in the set of probes specifically recognises at least one biomarker in the set of biomarkers; and
wherein the set of biomarkers comprises i) at least 7, 8, 9, 10, 11, 12, 13, 14, 15, 16, 17, 18, 19, or 20 biomarkers selected from Table 1:
TABLE 1

| | |
|---|---|
| Acrosomal protein SP-10 | Glial fibrillary acidic protein |
| Agouti-related protein | Immunoglobulin superfamily DCC |
| | subclass member 4 |
| CUB domain-containing protein 1 | Prostate-specific antigen |
| Collagen alpha-3(VI) chain | Kallikrein-7 |
| C-X-C motif chemokine 17 | Leukocyte cell-derived chemotaxin-2 |
| Tumor necrosis factor receptor | Latent-transforming growth factor |
| superfamily member 27 | beta-binding protein 2 |
| Elastin | Neurofilament light polypeptide |
| Endoglin | Podocalyxin-like protein 2 |
| Follitropin subunit beta | Receptor-type tyrosine-protein |
| | phosphatase R |
| Growth/differentiation factor 15 | Scavenger receptor class F member 2 |

or ii)
at least 50, 75, 100, 125, 150, 175, 200 or 204 biomarkers selected from Table 2:
TABLE 2

| | |
|---|---|
| Acrosomal protein SP-10 | PDZ domain-containing protein GIPC2 |
| Actin, aortic smooth muscle | Pancreatic secretory granule |
| | membrane major glycoprotein GP2 |
| Adenosine deaminase | Granzyme B |
| A disintegrin and metalloproteinase | Hepatitis A virus cellular receptor 1 |
| with thrombospondin motifs 13 | |
| A disintegrin and metalloproteinase | Hemicentin-2 |
| with thrombospondin motifs 15 | |
| A disintegrin and metalloproteinase | Corticosteroid 11-beta-dehydrogenase |
| with thrombospondin motifs 16 | isozyme 1 |
| ADAMTS-like protein 5 | Immunoglobulin superfamily DCC |
| | subclass member 4 |
| Adhesion G-protein coupled receptor | Interleukin-17D |
| G1 | |
| Alpha-fetoprotein | Interleukin-5 receptor subunit alpha |
| Advanced glycosylation end product- | Interleukin-7 receptor subunit alpha |
| specific receptor | |
| Agouti-related protein | Insulin-like 3 |
| Protein AHNAK2 | Integrin alpha-V |
| Angiopoietin-2 | Integrin beta-5 |
| BAG family molecular chaperone | Integrin beta-like protein 1 |
| regulator 3 | |
| Brevican core protein | Kinesin-like protein KIF22 |
| Osteocalcin | Mast/stem cell growth factor receptor |
| | Kit |
| Brother of CDO | Kallikrein-14 |
| Basigin | Prostate-specific antigen |
| Protein C19orf12 | Kallikrein-4 |
| Complement C1q-like protein 2 | Kallikrein-7 |
| Carbonic anhydrase 14 | Kallikrein-8 |
| Carbonic anhydrase 4 | Killer cell lectin-like receptor subfamily |
| | F member 1 |
| Calbindin | Neural cell adhesion molecule L1 |
| Coiled-coil domain-containing protein | Extracellular glycoprotein lacritin |
| 80 | |
| C-C motif chemokine 28 | Leukocyte cell-derived chemotaxin-2 |
| CCN family member 5 | Protein LEG1 homolog |
| T-cell surface glycoprotein CD1c | Lutropin subunit beta |
| Endosialin | Leiomodin-1 |
| T-cell surface glycoprotein CD8 alpha | Lactoperoxidase |
| chain | |
| Complement component C1q receptor | Latent-transforming growth factor beta- |
| | binding protein 2 |
| CUB domain-containing protein 1 | Ly6/PLAUR domain-containing protein |
| | 3 |
| Cadherin-2 | Apical endosomal glycoprotein |
| Cadherin-3 | Matrilin-3 |
| Cadherin-related family member 2 | Meprin A subunit beta |
| Cell adhesion molecule-related/down- | Matrix extracellular |
| regulated by oncogenes | phosphoglycoprotein |
| Cadherin EGF LAG seven-pass G-type | Tyrosine-protein kinase Mer |
| receptor 2 | |
| Complement factor H-related protein 5 | Lactadherin |
| Secretogranin-1 | Promotilin |
| Chitotriosidase-1 | Macrophage metalloelastase |
| Chordin-like protein 1 | Myelin-oligodendrocyte glycoprotein |
| Chordin-like protein 2 | Matrix remodeling-associated protein 8 |
| Cytoskeleton-associated protein 4 | Neurocan core protein |
| C-type lectin domain family 14 member | Neurofilament light polypeptide |
| A | |
| Contactin-5 | Nucleoside diphosphate kinase 3 |
| Collagen alpha-1(XV) chain | Neurogenic locus notch homolog |
| | protein 3 |
| Collagen alpha-3(VI) chain | N-acetylneuraminate lyase |
| Collagen alpha-1(IX) chain | Neuronal pentraxin-2 |
| Complement receptor type 2 | Neurotrophin-3 |
| Corticoliberin | Neurotrophin-4 |
| Cartilage acidic protein 1 | N-terminal prohormone of brain |
| | natriuretic peptide |
| Beta-crystallin B2 | Odontogenic ameloblast-associated |
| | protein |
| Chondroitin sulfate proteoglycan 5 | Glycodelin |
| Cystatin-SN | Inactive serine protease PAMR1 |
| Cystatin-D | phospholipase A2 inhibitor and |
| | Ly6/PLAUR domain-containing protein |
| Collagen triple helix repeat-containing | Polycystin-1 |
| protein 1 | |
| Cathepsin F | Tissue-type plasminogen activator |
| Cathepsin L2 | Podocalyxin-like protein 2 |
| Coxsackievirus and adenovirus | Pro-opiomelanocortin |
| receptor | |
| Stromal cell-derived factor 1 | Prolargin |
| C-X-C motif chemokine 14 | Prolactin |
| C-X-C motif chemokine 17 | Prion-like protein doppel |
| C-X-C motif chemokine 9 | Prokineticin-1 |
| NADH-cytochrome b5 reductase 2 | Persephin |
| Cytokine-like protein 1 | Prostaglandin-H2 D-isomerase |
| Discoidin, CUB and LCCL domain- | Pleiotrophin |
| containing protein 2 | |
| Decorin | Receptor-type tyrosine-protein |
| | phosphatase mu |
| Divergent protein kinase domain 2B | Receptor-type tyrosine-protein |
| | phosphatase N2 |
| Dickkopf-related protein 3 | Receptor-type tyrosine-protein |
| | phosphatase R |
| Dickkopf-like protein 1 | Receptor-type tyrosine-protein |
| | phosphatase zeta |
| Protein delta homolog 1 | Renin |
| Dentin matrix acidic phosphoprotein 1 | Proto-oncogene tyrosine-protein |
| | kinase receptor Ret |
| Dipeptidase 2 | Repulsive guidance molecule A |
| Dermatopontin | RGM domain family member B |
| Tumor necrosis factor receptor | Prorelaxin H2 |
| superfamily member 27 | |
| Epididymal secretory protein E3-beta | Roundabout homolog 1 |
| EGF-like repeat and discoidin I-like | Ribonucleoside-diphosphate reductase |
| domain-containing protein 3 | subunit M2 |
| EGF-containing fibulin-like extracellular | Scavenger receptor class F member 2 |
| matrix protein 1 | |
| EF-hand domain-containing protein D1 | Secretogranin-2 |
| Epidermal growth factor receptor | Secretogranin-3 |
| Elastin | Uteroglobin |
| Protein enabled homolog | Protein sidekick-2 |
| Endoglin | Neuronal-specific septin-3 |
| Beta-enolase | Superoxide dismutase [Mn], |
| | mitochondrial |
| Ectonucleotide | VPS10 domain-containing receptor |
| pyrophosphatase/phosphodiesterase | SorCS2 |
| family member 2 | |
| Ectonucleotide | Sclerostin |
| pyrophosphatase/phosphodiesterase | |
| family member 5 | |
| Receptor tyrosine-protein kinase erbB- | Serine protease inhibitor Kazal-type 1 |
| 4 | |
| Fatty acid-binding protein, adipocyte | Spondin-2 |
| Protein FAM3B | Small proline-rich protein 3 |
| Prolyl endopeptidase FAP | Sushi repeat-containing protein SRPX |
| Tumor necrosis factor receptor | Sushi domain-containing protein 2 |
| superfamily member 6 | |
| Tumor necrosis factor ligand | Sushi domain-containing protein 5 |
| superfamily member 6 | |
| Fibulin-2 | Trefoil factor 1 |
| Fc receptor-like protein 2 | Thrombospondin-2 |
| Fibroblast growth factor 5 | Tumor necrosis factor receptor |
| | superfamily member 11B |
| Follitropin subunit beta | Tumor necrosis factor receptor |
| | superfamily member 13B |
| Follistatin-related protein 1 | Tumor necrosis factor ligand |
| | superfamily member 13 |
| Growth arrest-specific protein 6 | Tenascin-X |
| Growth/differentiation factor 15 | Tetraspanin-1 |
| Glial fibrillary acidic protein | WAP four-disulfide core domain protein |
| | 2 |
| GDNF family receptor alpha-like | Wnt inhibitory factor 1 |
| Appetite-regulating hormone | Protein Wnt-9a |
| Gastric inhibitory polypeptide | Lymphotactin |

13. The set of probes of claim 12, wherein each probe in the set is independently selected from the group consisting of an antibody, antibody fragment, oligonucleotide, protein, biotin-binding protein, enzyme, and fluorophore, or a combination thereof.

14. The set of probes of claim 12, wherein the set of biomarkers comprises at least 7, 8, 9 or 10 biomarkers selected from Table 3:
TABLE 3

| | |
|---|---|
| Tumor necrosis factor receptor | Elastin |
| superfamily member 27 | |
| Collagen alpha-3(VI) chain | Immunoglobulin superfamily DCC |
| | subclass member 4 |
| Growth/differentiation factor 15 | Follitropin subunit beta |
| Neurofilament light polypeptide | Latent-transforming growth factor beta- |
| | binding protein 2 |
| Podocalyxin-like protein 2 | Prostate-specific antigen. |

15. A device for determining the presence or amount of each biomarker in a set of biomarkers;
wherein the device comprises a set of probes according to claim 12, preferably wherein each probe is independently selected from the group consisting of an antibody, antibody fragment, oligonucleotide, protein, biotin-binding protein, enzyme, and fluorophore, or a combination thereof.
